(19) United States
(12) Patent Application Publication
(10) Pub. No.: US 2005/0148506 A1
(43) Pub. Date: Jul. 7, 2005

(54) Novel method of modulating bone-related activity

(75) Inventors: Julia Billiard, Collegeville, PA (US); Peter V.N. Bodine, Havertown, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 10/823,998

(22) Filed: Apr. 14, 2004

Related U.S. Application Data
(60) Provisional application No. 60/463,364, filed on Apr. 16, 2003.
(60) Provisional application No. 60/501,340, filed on Sep. 9, 2003.

(51) Int. Cl. C12Q 1/68; C07H 21/04; C12N 9/64; A61K 38/17

(52) U.S. Cl. 514/12; 435/6; 435/69.1; 435/320.1; 435/226; 435/325; 536/23.2

(57) ABSTRACT

This invention relates to modulating bone-related activity in a subject by modulating Ror molecules. The invention further relates to compositions and methods for the screening, diagnosis and development of therapies for bone-related disorders. It is emphasized that this abstract is provided to comply with the rules requiring an abstract that will allow a searcher or other reader to quickly ascertain the subject matter of the technical disclosure. It is submitted with the understanding that it will not be used to interpret or limit the scope or meaning of the claims.

### CROSS-REFERENCE TO RELATED APPLICATIONS

This application claims the benefit under 35 U.S.C. §119(e) to U.S. provisional applications 60/463,364 filed Apr. 16, 2003, and 60/501,340, filed Sep. 9, 2003, both of which are hereby incorporated by reference in their entireties.

### FIELD OF THE INVENTION

The invention is in the field of molecular biology. More specifically, the invention pertains to methods and compositions for the diagnosis, prognosis, prevention, treatment, and evaluation of therapies for bone-related disorders.

### BACKGROUND OF THE INVENTION

The topic of bone-related disorders and diseases has gained considerable attention over the past years. Bone-related disorders are characterized by bone loss resulting from an imbalance between bone resorption and bone formation. Throughout life, there is a constant remodeling of skeletal bone. In this remodeling process, there is a delicate balance between bone resorption by osteoclasts and subsequent restoration by osteoblasts. Osteoblasts, involved in both endochondral and intramembranous ossification, are the specialized cells in bone tissue that make matrix proteins resulting in the formation of new bone. Bone formation, i.e. osteogenesis, is essential for the maintenance of bone mass in the skeleton. Unlike osteoblasts, osteoclasts are associated with bone resorption and removal. In normal bone, the balance between osteoblast-mediated bone formation and osteoclast-mediated bone resorption is maintained through complex regulated interactions.

There are many deficiencies, diseases, and disorders associated with the skeletal system. Examples of a few include, but are not limited to, osteoporosis, bone cancer, arthritis, rickets, bone fracture, periodontal disease, bone segmental defects, osteolytic bone disease, primary and secondary hyperparathyroidism, Paget's disease, osteomalacia, hyperostosis, and osteopetrosis. Identification of the mechanisms involved in osteogenic differentiation and the renewal processes are crucial for the understanding of bone physiology and skeletal disorders, such as osteoporosis. These disorders may involve deficient bone formation due to defective maturation of putative osteoblastic progenitors.

There exists a need to develop methods of treating diseases or disorders associated with bone growth, methods of hastening bone formation, methods of identifying agents that modulate (increase or decrease) bone formation, and methods of identifying genes or their protein products associated with bone related disorders.

Identification of the mechanisms involved in bone formation and bone resorption are crucial for the understanding of bone physiology and skeletal disorders, such as osteoporosis. The genes or their protein products which are associated with bone related disorders may be used for the elucidation of the molecular mechanisms of bone formation, bone resorption, for the screening and development of new drugs, for diagnosis, prognosis, prevention, and treatment of bone development and bone loss disorders, and evaluation of therapies for bone-related disorders such as osteoporosis.

The present invention not only provides a method for modulating bone related activity, it also provides a method by which the understanding of the mechanisms involved in bone formation and bone resorption are furthered.

### SUMMARY OF THE INVENTION

The present invention relates to an expression cassette comprising a polynucleoticle encoding a Ror polypeptide or homologues or derivatives or fragments or variants or mutants thereof wherein said polynucleotide is under the control of a promoter operable in bone cells.

The present invention relates to a host cell comprising an expression cassette comprising a polynucleotide encoding a Ror polypeptide or homologues or derivatives or fragments or variants or mutants thereof, wherein said polynucleotide is under the control of a promoter operable in eukaryotic cells, said promoter being heterologous to said polynucleotide.

The present invention relates to a composition for modulating bone-related activity comprising an effective amount of Ror molecule or homologues or derivatives or fragments or variants or mutants thereof.

The present invention relates to a method of screening for agents, the method comprising: (a) combining an agent with a Ror molecule; and (b) detecting an effect of said agent on Ror activity; wherein detection of a decrease or an increase in Ror activity is indicative of an agent being a bone-related agent.

The present invention relates to a method of screening for agents, the method comprising: (a) combining an agent with an isolated cell comprising a Ror promoter sequence operatively linked to a reporter gene; and (b) detecting an effect of said agent on reporter activity; wherein detection of a decrease or an increase in Ror promoter activity as measured by the reporter activity is indicative of an agent being a bone-related agent.

The present invention relates to a method of screening for agents that modulate the binding of Ror polypeptide to a binding partner comprising: (a) contacting Ror polypeptide with a Ror binding partner in the presence of an agent; (b) contacting Ror polypeptide with a Ror binding partner in the presence of a control or in the absence of the agent; and (c) selecting the agent that modulates Ror polypeptide binding to Ror binding partner by comparing the binding of said Ror polypeptide to the binding partner in step (a) to the binding of said Ror polypeptide to a binding partner in step (b).

The present invention relates to a method of modulating bone-related activity in a subject comprising administering to a subject an agent which modulates target Ror molecule expression or activity.

The present invention relates to a method of modulating Wnt-1 and Wnt-3 activity in a subject comprising administering an agent which modulates target Ror2 molecule expression or activity in an amount effective to regulate Wnt-1 and Wnt-3 activity.

The present invention relates to a method of modulating Wnt-3 activity in a subject comprising administering an agent which modulates target Ror1 polypeptide expression or activity in an amount effective to regulate Wnt-3 activity.

The present invention relates to a method for identifying an agent for modulating bone-related activity comprising: (a) expressing Ror molecule in a cell or using endogenous Ror expression; (b) contacting the cell with the agent; and (c) monitoring the expression or the activity of Ror molecule wherein an increase or decrease in the expression or activity of Ror molecule in the presence of the agent identifies the agent as modulating bone-related activity.

The present invention relates to a method for identifying an agent for modulating Wnt signaling pathway comprising: screening one or more agents for the ability to modulate expression or activity of Ror molecule, wherein the agent that can modulate expression or activity of Ror molecule is an agent that modulates Wnt signaling pathway.

The present invention relates to a method of linking a bioactive molecule to a cell expressing a Wnt polypeptide, said method comprising contacting said cell with a Ror2 polypeptide that is bound to said bioactive molecule and allowing said Wnt polypeptide and said Ror2 polypeptide to bind to one another, thereby linking said bioactive molecule to said cell.

The present invention relates to a method for screening a subject for a bone-related disorder comprising the steps of: measuring the expression of Ror molecule in a subject and determining the relative expression of said Ror molecule in the subject compared to its expression in normal subjects, or compared to its expression in the same subject after being treated for bone-related disorders.

The present invention relates to a method for screening a subject for a bone-related disorder comprising the steps of: measuring the activity of Ror polypeptide in a subject and determining the relative activity of said Ror polypeptide in the subject compared to its activity in normal subjects, or compared to its activity in the same subject after being treated for bone-related disorders.

The present invention relates to a method of identifying genes that participate in bone formation comprising: a) overexpressing Ror molecule in a cell, b) monitoring the changes in gene expression profile and c) determining which genes are regulated by Ror expression thereby identifying genes that participate in bone formation.

The present invention relates to a method of identifying genes that modulate Wnt signaling pathway comprising: a) overexpressing Ror molecule in a cell, b) monitoring the changes in gene expression profile and c) determining which genes are regulated by Ror expression thereby identifying genes that modulate Wnt signaling pathway.

The present invention relates to a method for identifying proliferating human pre-osteoblastic cells using Ror2 as a marker, comprising determining expression of Ror2 gene in a human osteoblastic cell wherein the increased Ror2 expression identifies the cell as being proliferating pre-osteoblastic cells.

The present invention relates to a method for identifying mouse osteoblastic cells at the stage of matrix maturation using Ror2 as a marker, comprising determining expression of Ror2 gene in a mouse osteoblastic cell wherein the increased Ror2 expression identifies the cell as being an osteoblastic cell at the stage of matrix maturation.

The present invention relates to a method of determining Ror 2 kinase activity comprising: (a) obtaining Ror2 polypeptide; (b) labeling Ror2 polypeptide in presence of ³²P γ ATP; (c) determining Ror2 kinase activity by measuring the amount of incorporated ³²P wherein the amount of ³²P indicates activity of Ror2 kinase.

### BRIEF DESCRIPTION OF THE DRAWINGS AND SEQUENCE DESCRIPTIONS

The invention can be more fully understood from the following detailed description and the accompanying drawings and Sequence Listing that form a part of this application.

FIG. 1 shows that the expression of Ror kinases decreases during late stages of human osteoblast differentiation. Expression of Ror2 (FIG. 1A) and Ror1 (FIG. 1B) in cells representing different stages of osteoblast differentiation from pre-osteoblasts to mature osteocytes was assessed by gene chip analysis on GIHuman1a chip (circles) and by real-time RT-PCR (bars). For both methods, the relative mRNA expression in HOB-03-C5 was set at one. Real-time RT-PCR was performed using probes and primers listed in Table 1 in Example 1. The levels of mRNA were normalized to the expression of 18S rRNA in each sample. Means±Standard Errors (SE) of three RT-PCR reactions per cell line. Ror2 expression in HOB-05-T1 cells was undetectable by RT-PCR. Cell lines: 03-C5-HOB-03-C5, pre-osteoblasts, 03-CE6-HOB-03-CE6, early osteoblasts; 02-C1-HOB-02-C1, mature osteoblasts; 01-C1-HOB-01-C1, pre-osteocytes; 05-T1-HOB-05-T1, mature osteocytes (FIG. 1 is referred to in Example 1).

FIG. 2 shows that SFRP-1 suppresses Ror2 expression. Results of the gene chip analysis of Ror2 (closed bars) and Ror1 (open bars) expression on GIHuman1a chip using polyA⁽⁺⁾RNA from HOB-01-09 osteocytes stably overexpressing SFRP-1 (01-09SFRP-1) or empty vector (01-09 vector) or from calvarial bones of wild type or SFRP-1 −/− mice. Levels of Ror1 expression in wild type and SFRP1−/− mice were below the chip detection limits. The relative mRNA expression in the 01-09vector cells and in wild type mice was set at one (FIG. 2 is referred to in Example 1).

FIG. 3 shows a predicted domain structure of Ror proteins. The domains are: IG—immunoglobulin; FRZ—frizzled; kringle—triple loop structure linked by three pairs of disulfide bonds originally found in prothrombin; M—transmembrane; Tyr Kin—tyrosine kinase. B. Localization of disulfide bonds in the frizzled module of Ror1 (Roszmusz, et al., E., 2001, Journal of Biological Chemistry. 276: 18485-90). Numbers refer to the 10 conserved cysteines in the frizzled domain (FIG. 3 is referred to in Example 1).

FIG. 4 shows that the expression of Ror2, but nor Ror1, kinase increases during early stages of human osteoblast differentiation and through later stages of mouse osteoblast differentiation. A. Human MSC were incubated in human osteogenic medium (0.1 mM dexamethasone, 0.05 mM ascorbic acid and 10 mM β-glycerophosphate in growth medium) and total cellular RNA was collected at times indicated and analyzed by RT-PCR for expression of Ror1 and Ror2 genes. B. Murine MC3T3-E1 cells were incubated in mouse osteogenic medium (25 μg/ml ascorbic acid and 10 mM β-glycerophosphate in growth medium) and total cellular RNA was collected at times indicated and analyzed by RT-PCR for expression of Ror1, Ror2, alkaline phosphatase (AP), and osteocalcin (OC) genes (FIG. 3 is referred to in Example 2).

FIG. 5 shows expression of Ror proteins in U2OS cells. A. Schematic representations of the full-length Ror1 and Ror2 kinases and the Ror2 mutants. FLAG—flag epitope tag; M—transmembrane domain; Tyr Kin—tyrosine kinase domain. B. Western immunoblot for the flag epitope tag of the whole-cell protein extracts (50 μg/lane) from U2OS cells transfected with the indicated Ror constructs. Position of Ror1, Ror2 and Ror2KD is marked by an arrowhead and Ror2ΔC-flag, by an arrow. C. The top panel shows an autoradiograph of the results of in vitro autophosphorylation assay performed as described under General Methods using Ror2-flag or Ror2KD-flag immunoprecipitated on flag affinity agarose. In the bottom panel, ten percent of the flag immunoprecipitated proteins were separated by SDS-PAGE and analyzed by Silver staining to assess kinase levels in the autophosphorylation reactions (FIG. 5 is referred to in Examples 3, 6, and 8).

FIG. 6 shows that Ror2 kinase inhibits Wnt-3, but potentiates Wnt-1 activity. U2OS cultures were transiently tranfected with a recombinant luciferase reporter gene containing 16 copies of the TCF binding site cloned 5′ to the thymidine kinase promoter. In A, the promoter-reporter gene was co-transfected with pcDNA3.1(+) (vector), Ror2-flag (R2), Wnt-3-HA (w3), or Wnt-3-HA plus the indicated amounts of Ror2-flag (in ng per well of a 96-well plate) or SFRP-1 (S) or both. In B, the promoter-reporter gene was co-transfected with pcDNA3.1(+), Wnt-1-HA (w1), or Wnt-1-HA plus the indicated amounts of Ror2-flag or SFRP-1 or both. Luciferase values measured after transfection of a reporter gene in presence of pcDNA3.1(+) have been arbitrarily given a value of 1. In A, the results are means±SE of at least two independent experiments with n≧16 and the asterisks indicate significant decreases in luciferase activity below the level obtained with Wnt-3-HA alone (*-p<0.05, **-p<0.0001). In B, the results are means±SE of at least three independent experiments with n≧24 and the asterisks indicate significant increases in luciferase activity above the level in presence of Wnt-1-HA alone (*-p<0.05, **-p<0.0001) (FIG. 6 is referred to in Example 4).

FIG. 7 shows that Ror1 kinase inhibits Wnt-3, but has no effect on Wnt-1 activity. As in FIG. 6 except Ror1-flag (R1) was used in place of Ror2-flag. Means±SE, n≧8. In A, the asterisks indicate significant decreases in luciferase activity below the level obtained with Wnt-3-HA alone (*-p<0.01, **-p<0.0001) (FIG. 7 is referred to in Example 5).

FIG. 8 shows that the tyrosine kinase activity of Ror2 is required for potentiation of Wnt-1 and for most of inhibition of Wnt-3 activity. As in FIG. 6 except Ror2KD-flag (R2 KD) was used in place of Ror2-flag. In A and B, the results are means±SE of at least three independent experiments with n≧24. The asterisks indicate significant decreases in luciferase activity below the level obtained with Wnt-3-HA alone (**-p<0.0001) (FIG. 8 is referred to in Example 6).

FIG. 9 shows that the cytoplasmic domain of Ror2 is required for potentiation of Wnt-1 and for part of inhibition of Wnt-3 activity. As in FIG. 6 except Ror2ΔC-flag (R2d) was used in place of Ror2-flag. In A, the results are means±SE of at least two independent experiments with n≧16. The asterisks indicate significant decreases in luciferase activity below the level obtained with Wnt-3-HA alone (*-p<0.05, **-p<0.0001). In B, n≧8 (FIG. 9 is referred to in Example 6).

FIG. 10 shows that Ror2 and Ror2KD bind to Wnt-1 and Wnt-3. COS7 cells were transiently transfected with vector controls or the indicated combinations of Ror2-flag and Wnts-HA. The total amount of DNA was kept constant by addition of pcDNA3.1(+) or pUSEamp in place of Ror2 or Wnts, respectively. At 24 h, lysates were analyzed by SDS-PAGE, directly (top) or after immunoprecipitation with anti-flag antibody (bottom). Immunoblotting was performed with anti-HA antibody (FIG. 10 is referred to in Example 7).

FIG. 11 shows that Ror2 discriminates between different Wnts. COS7 cells were transiently transfected with the indicated Wnts-HA and Ror2-Flag (+) or pcDNA3.1. At 24 h, lysates were immunoprecipitated with anti-flag antibody and analyzed for presence of Wnts by anti-HA antibody (top). The bottom panel shows western blot analysis with anti-HA antibody of the COS7 extracts containing the indicated Wnts and the Ror2-flag (control for equal loading in the immunoprecipitation reactions) (FIG. 11 is referred to in Example 7).

FIG. 12 shows that overexpression of Wnt-1 and Wnt-3 has no effect on the extent of Ror2 autophosphorylation. A. The top panel shows an autoradiograph of the results of in vitro autophosphorylation assay performed as described under General Methods using Ror2-flag immunoprecipitated on flag affinity agarose out of U2OS cells co-transfected with Ror2-flag and the indicated Wnts. The bottom panel shows western immunoblotting of the same membrane with anti-flag antibody. B. Autoradiographic signals were normalized to the total amount of immunoreactive Ror2 protein in each reaction and the relative signal obtained in absence of Wnts was set at one. Means±SE of three independent experiments (FIG. 12 is referred to in Example 8).

FIG. 13 shows that Ror2 inhibits Wnt-mediated stabilization of cytosolic β-catenin. U2OS cultures were transiently transfected with the indicated combinations of Ror2 (R2) and Wnts (W). The total amount of DNA was kept constant by addition of pcDNA3.1(+) or pUSEamp in place of Ror2 or Wnts, respectively. At 24 h, cytoplasmic proteins were analyzed by western immunoblotting with anti-β-catenin antibody. The levels of β-catenin were normalized to the signal obtained in absence of Wnts after equal loading was verified by staining with anti-β-actin antibody. The numbers are means of three independent experiments (FIG. 13 is referred to in Example 9).

FIG. 14 shows a proposed model for Ror2 activity whereby Ror2 binds both Wnts, sequestering them away from Frizzled receptors and inhibiting their ability to stabilize β-catenin. In addition, Wnt1 binding to the Ror2 receptor causes activation of an unidentified signaling cascade that requires tyrosine kinase activity of the Ror2 receptor and results in potentiation of Wnt-responsive promoter activity. Wnt3 binding does not stimulate the same cascade, but instead activates other tyrosine kinase-dependent events that lead to inhibition of Wnt-responsive promoters. FZ—Frizzled receptor, GSK-3β-glycogen synthase kinase β, β-cat-β-catenin, Lef/Tcf—lymphoid-enhancer binding factor/T-cell transcription factor (FIG. 14 is referred to in Example 10).

FIG. 15 illustrates identification of the Ror2 binding partners in U2OS cells. Whole-cell extracts from U2OS cells transiently transfected with the constructs identified on top were immunoprecipitated with anti-flag antibody and subjected to SDS-PAGE analysis on 4-12% (A) or 7% (B) polyacrylamide gels. In parallel experiments, the 4-12% gels were transblotted onto nitrocellulose membrane and western immunoblotting was performed with anti-flag (C) or anti-phosphotyrosine (D) antibody. Arrows point to bands that appear to be Ror2-dependent. M—molecular weight in kDa. The prominent bands around 55 and 25 kDa are IgG subunits dissociated from the flag affinity agarose (FIG. 15 is referred to in Example 11).

FIG. 16 shows that Ror2 binds to the intracellular domain of Notch2 (Notch2IC). U2OS cells were transiently transfected with vector controls or the indicated combinations of Ror2-flag and Notch2IC-V5-his. The total amount of DNA was kept constant by addition of pcDNA3.1(+). At 24 h, lysates were analyzed by SDS-PAGE directly (top) or after immunoprecipitation with anti-flag antibody. Immunoblotting was performed with anti-V5 (top) or anti-his (bottom) antibody (FIG. 16 is referred to in Example 12).

FIG. 17 confirms generation of cell lines stably over-expressing Ror2 and Ror1. A. Relative Ror2 and Ror1 mRNA expression in HOB-01-09 cells over-expressing Ror2, Ror2-Flag, Ror1-Flag, or empty vector (pcDNA 3.1(+)). The relative mRNA expression in HOB-01-09-pcDNA cells was set at one. Real-time RT-PCR was performed using probes and primers listed in Table 1 in Example 1. The levels of mRNA were normalized to the expression of 18S rRNA in each sample. B. Western immunoblot with the indicated antibodies of the whole-cell protein extracts (50 μg/lane) from HOB-01-09 cells over-expressing Ror2, Ror2-Flag, Ror1-Flag, or empty vector (FIG. 17 is referred to in Example 13).

Table 1 shows primers and probes used in the real-time RT-PCR analysis of human Ror mRNA (Table 1 is referred to in Example 1).

Table 2 shows primers and probes used in the real-time RT-PCR analysis of mouse Ror mRNA (Table 2 is referred to in Example 2).

Table 3 shows primers and probes used in the real-time RT-PCR analysis of mouse alkaline phosphatase and osteocalcin mRNA (Table 3 is referred to in Example 2).

Table 4 shows potential Ror2 interacting proteins (Table 4 is referred to in Example 9).

The following 48 sequence descriptions and sequence listings attached hereto comply with the rules governing nucleotide and/or amino acid sequence disclosures in patent applications as set forth in 37 C.F.R. §1.821-1.825. (“Requirements for Patent Applications containing nucleotide sequences and/or Amino Acid Sequence Disclosure—the Sequence Rules”) and consistent with World Intellectual Property Organization (WIPO) Standard ST.25 (1998) and the sequence listing requirements of the EPO and PCT (Rules 5.2 and 4.95(a-bis) and Section 208 and Annex C of the Administrative Instructions). The Sequence Descriptions contains the one letter code for nucleotide sequence characters and the three letter codes for amino acids as defined in conformity with the IUPAC-IUBMB standards described in Nucleic Acids Res., 13, 3021-3030, (1985) and in the Biochemical J., 219(2), 345-373, (1984) which are herein incorporated by reference. The symbols and format used for nucleotide and amino acid sequence data comply with the rules set forth in 37 C.F.R. §1.822.

SEQ ID NO:1 is the first nucleotide sequence containing the TCF DNA binding sites originally identified in the TCR-alpha enhancer, the CD3-e enhancer, and the consensus TCF DNA binding site.

SEQ ID NO:2 is the second nucleotide sequence containing the TCF DNA binding sites originally identified in the TCR-alpha enhancer, the CD3-e enhancer, and the consensus TCF DNA binding site.

SEQ ID NO:3 is the nucleotide sequence that codes for Ror1 protein.

SEQ ID NO:4 is the deduced amino acid sequence of Ror1 protein.

SEQ ID NO:5 is the nucleotide sequence that codes for Ror2 protein.

SEQ ID NO:6 is the deduced amino acid sequence of Ror2 protein.

SEQ ID NO:7 is the nucleotide sequence that codes for Ror1-flag protein.

SEQ ID NO:8 is the deduced amino acid sequence of Ror1-flag protein.

SEQ ID NO:9 is the nucleotide sequence that codes for Ror2-flag protein.

SEQ ID NO:10 is the deduced amino acid sequence of Ror2-flag protein.

SEQ ID NO:11 is the nucleotide sequence that codes for Ror2ΔC-flag protein.

SEQ ID NO:12 is the deduced amino acid sequence of Ror2ΔC-flag protein.

SEQ ID NO: 13 is the nucleotide sequence of the top strand primer used to construct Ror1-flag.

SEQ ID NO: 14 is the nucleotide sequence of the bottom strand primer used to construct Ror1-flag.

SEQ ID NO:15 is the nucleotide sequence of the first top strand primer used to construct Ror2-flag.

SEQ ID NO:16 is the nucleotide sequence of the first bottom strand primer used to construct Ror2-flag.

SEQ ID NO:17 is the nucleotide sequence of the second top strand primer used to construct Ror2-flag.

SEQ ID NO:18 is the nucleotide sequence of the second bottom strand primer used to construct Ror2-flag.

SEQ ID NO:19 is the nucleotide sequence of the third top strand primer used to construct Ror2-flag.

SEQ ID NO:20 is the nucleotide sequence of the third bottom strand primer used to construct Ror2-flag.

SEQ ID NO:21 is the nucleotide sequence of the top strand primer used to construct Ror2KD-flag.

SEQ ID NO:22 is the nucleotide sequence of the bottom strand primer used to construct Ror2KD-flag.

SEQ ID NO:23 is the nucleotide sequence of the top strand primer used to construct Ror2ΔC-flag.

SEQ ID NO:24 is the nucleotide sequence of the bottom strand primer used to construct Ror2ΔC-flag.

SEQ ID NO:25 is the nucleotide sequence of the forward primer to identify human Ror1 (2993-3013).

SEQ ID NO:26 is the nucleotide sequence of the reverse primer to identify human Ror1 (3049-3074).

SEQ ID NO:27 is the nucleotide sequence of the probe to identify human Ror1 (3018-3044).

SEQ ID NO:28 is the nucleotide sequence of the forward primer to identify human Ror2 (1149-1169).

SEQ ID NO:29 is the nucleotide sequence of the reverse primer to identify human Ror2 (1239-1259).

SEQ ID NO:30 is the nucleotide sequence of the probe to identify human Ror2 (1174-1198).

SEQ ID NO:31 is the nucleotide sequence of the forward primer to identify mouse Ror1 (2350-2370).

SEQ ID NO:32 is the nucleotide sequence of the reverse primer to identify mouse Ror1 (2402-2421).

SEQ ID NO:33 is the nucleotide sequence of the probe to identify mouse Ror1 (2372-2395).

SEQ ID NO:34 is the nucleotide sequence of the forward primer to identify mouse Ror2 (364-386).

SEQ ID NO:35 is the nucleotide sequence of the reverse primer to identify mouse Ror2 (429-448).

SEQ ID NO:36 is the nucleotide sequence of the probe to identify mouse Ror2 (400-424).

SEQ ID NO:37 is the nucleotide sequence of the forward primer to identify mouse alkaline phosphatase (1354-1373).

SEQ ID NO:38 is the nucleotide sequence of the reverse primer to identify mouse alkaline phosphatase (1445-1464).

SEQ ID NO:39 is the nucleotide sequence of the probe to identify mouse alkaline phosphatase (1416-1442).

SEQ ID NO:40 is the nucleotide sequence of the forward primer to identify mouse osteocalcin (78-96).

SEQ ID NO:41 is the nucleotide sequence of the reverse primer to identify mouse osteocalcin (124-145).

SEQ ID NO:42 is the nucleotide sequence of the probe to identify mouse osteocalcin (98-121).

SEQ ID NO:43 is the nucleotide sequence of the 5′ untranslated region of the human Ror1 gene (−2000 to +1).

SEQ ID NO:44 is the nucleotide sequence of the 5′ untranslated region of the mouse Ror2 gene (−2000 to +1).

SEQ ID NO:45 is the nucleotide sequence of the top strand primer used to construct the 5′ portion of Notch2IC (1-782).

SEQ ID NO:46 is the nucleotide sequence of the bottom strand primer used to construct the 5′ portion of Notch2IC (1-782).

SEQ ID NO:47 is the nucleotide sequence of the top strand primer used to construct the 3′ portion of Notch2IC (783-2307).

SEQ ID NO:48 is the nucleotide sequence of the bottom strand primer used to construct the 3′ portion of Notch2IC (783-2307).

### DETAILED DESCRIPTION OF THE INVENTION

Applicants have discovered that expression of genes coding for human receptor tyrosine kinase-like orphan receptors 1 and 2 (Ror1 and Ror2) is significantly down-regulated during human osteoblast differentiation. Applicants have also provided evidence that Ror2 expression is inversely related to the expression of secreted frizzled-related protein 1 (SFRP-1). SFRP-1 has been reported to be a potential osteoporosis target that stimulates apoptosis of osteoblasts in vitro and in vivo (WO 01/19855).

Moreover, Applicants discovered that in osteoblastic cells, Ror1 and Ror2 modulate Wnt signaling pathways that regulate survival of bone-forming osteoblasts. In one embodiment, Ror2 kinase inhibits Wnt-3 but potentiates Wnt-1 activity. Furthermore, Ror2 binds to both Wnt-1 and Wnt-3 proteins. In another embodiment, the cytoplasmic domain of Ror2 is required for potentiation of Wnt-1 but not for inhibition of Wnt-3 activity. In yet another embodiment, Ror1 kinase inhibits Wnt-3 but has no effect on Wnt-1 activity.

Several Ror2 binding partners have also been identified.

The present invention provides an expression cassette comprising Ror polypeptide under the control of a promoter operable in bone cells. The present invention further provides a composition for modulating bone-related activity comprising effective amount of Ror molecule or homologues or derivatives or fragments or variants or mutants thereof.

The present invention provides for a method of screening for agents, the method comprising: (a) combining a agent with a Ror molecule; and (b) detecting an effect of said agent on Ror activity; wherein detection of a decrease or an increase in Ror activity is indicative of an agent being a bone-related agent.

Moreover, the present invention provides for a method of screening for agents that modulate the binding of Ror to a binding partner comprising: (a) contacting Ror with the Ror binding partner in the presence of an agent; (b) contacting Ror with a Ror binding partner in the presence of a control or in the absence of the agent and, (c) selecting the agent that modulates Ror molecule by comparing the binding of said Ror to the binding partner in step (a) to the binding of said Ror to the binding partner in step (b).

The method can also include a method of screening for agents, the method comprising: (a) combining an agent with an isolated cell comprising a Ror promoter sequence operatively linked to a reporter gene; and (b) detecting an effect of said agent on Ror activity; wherein detection of a decrease or an increase in Ror activity as measured by the reporter is indicative of an agent being a bone-related agent.

The present invention provides methods of administering an agent identified to modulate Ror expression or activity in the form of a pharmaceutical composition to subjects to treat bone-related disorders. Moreover, the present invention provides methods of administering an agent identified to modulate Ror expression to treat subjects diagnosed with diseases or conditions associated with Wnt signaling pathway.

The present invention further provides for the use of an effective amount of an agent which modulates target Ror molecule expression or activity in the preparation of a composition for modulating bone-related activity in the treatment of bone related disorders. Preferably the agent is identified by a screening method provided therein.

The agent which modulates target Ror molecule expression or activity used in the preparation of the composition may be selected from the group consisting of an antibody, a small molecule, a peptide, an oligopeptide and a polypeptide.

Preferably an agent for use in accordance with the invention comprises an antisense nucleic acid or siRNA molecule specific for Ror gene, wherein said antisense nucleic acid or siRNA molecule recognizes and binds to a nucleic acid encoding one or more Ror polypeptides, homologues, derivatives, fragments, variants or mutants thereof.

In an alternative embodiment an agent for use in accordance with the present invention to modulate Ror molecule expression or activity is capable of binding to a Ror binding partner.

Where the target Ror module is Ror2 the agent for use in the preparation of a composition according to the invention may be selected from the group consisting of ADP/ATP carrier protein, UDP-glucose ceramide glucosyltransferase-like 1, 14-3-3 protein beta/alpha, 14-3-3 protein gamma, ribophorin I, arginine N-methyltransferase 1, cellular apoptosis susceptibility protein, NOTCH2 protein, and human skeletal muscle LIM-protein 3.

The present invention further provides a method of preparing a composition for modulating bone-related activity wherein said method comprises:
(i) identifying a bone-related agent by combining an agent with a Ror molecule and detecting an effect of said agent on Ror activity
(ii) combining an effective amount of the bone-related agent identified in step (i) with a pharmaceutically acceptable carrier to form said composition.

The present invention also provides additional uses of the Ror molecules, such as identifying agents that modulate bone formation and/or Wnt signaling, identifying genes or proteins that participate in bone formation and/or Wnt signaling, diagnostic uses, uses as pharmaceutical drug targets, evaluating the efficacy of drugs, and generating host cells and transgenic animals.

The present provides for a method of identifying proliferating human pre-osteoblastic cells using Ror2 as a marker, comprising determining expression of Ror2 gene in a human osteoblastic cell wherein the increased Ror2 expression identifies the cell as being proliferating pre-osteoblastic cells.

The present invention further provides for a method of identifying mouse osteoblastic cells at the stage of matrix maturation using Ror2 as a marker, comprising determining expression of Ror2 gene in a mouse osteoblastic cell wherein the increased Ror2 expression identifies the cell as being an osteoblastic cell at the stage of matrix maturation.

In addition, the present invention provides for a of determining Ror 2 kinase activity comprising: (a) obtaining Ror2 polypeptide; (b) labeling Ror2 polypeptide in presence of ³²P γ ATP; (c) determining Ror2 kinase activity by measuring the amount of incorporated ³²P wherein the amount of ³²P indicates activity of Ror2 kinase.

Definitions of Abbreviations and Terms:

The following definitions are provided for the full understanding of terms and abbreviations used in this specification.

As used herein and in the appended claims, the singular forms “a”, “an”, and “the” include the plural reference unless the context clearly indicates otherwise. Thus, for example, a reference to “a host cell” includes a plurality of such host cells, and a reference to “an antibody” is a reference to one or more antibodies and equivalents thereof known to those skilled in the art, and so forth.

The abbreviations in the specification correspond to units of measure, techniques, properties or compounds as follows: “g” means gram(s), “mg” means milligram(s), “ng” means nanogram(s), “kDa” means kilodalton(s), “° C.” means degree(s) Celsius, “cm” means centimeter(s), “s” means second(s), “min” means minute(s), “h” means hour(s), “l” means liter(s), “ml” means milliliter(s), “μl” means microliter(s), “pl” means picoliter(s), “M” means molar, “mM” means millimolar, “mmole” means millimole(s), “kb” means kilobase(s), “bp” means base pair(s), and “RT” means room temperature.

“Dulbecco's-modified Eagle Medium” is abbreviated DMEM.

“High performance liquid chromatography” is abbreviated HPLC.

“High throughput screening” is abbreviated HTS.

“Open reading frame” is abbreviated ORF.

“Mass-spectroscopy” is abbreviated MS.

“Tandem mass-spectroscopy” is abbreviated MS/MS.

“Polyacrylamide gel electrophoresis” is abbreviated PAGE.

“Polymerase chain reaction” is abbreviated PCR.

“Reverse transcriptase polymerase chain reaction” is abbreviated RT-PCR.

“Sodium dodecyl sulfate” is abbreviated SDS.

“Sodium dodecyl sulfate-polyacrylamide gel electrophoresis” is abbreviated SDS-PAGE.

“Human skeletal muscle LIM-protein 3” is abbreviated (SLIM 3).

“Adenine nucleotide translocator 2” is abbreviated ADP/ATP carrier protein.

“Bone Mineral Density” is abbreviated BMD.

“Ribosomal RNA” is abbreviated rRNA”.

“Untranslated region” is abbreviated UTR.

“T-cell factor” is abbreviated TCF.

“Dithiothreitol” is abbreviated DTT.

In the context of this disclosure, a number of terms shall be utilized. As used herein, the term Ror refers to a family of receptor tyrosine kinase-like orphan receptors. “Ror molecule” refers to Ror polypeptides, Ror peptides, fragments, variants, and mutants thereof as well as to nucleic acids that encode Ror polypeptides, Ror peptides and fragments or variants or mutants thereof. “Ror molecule” also refers to Ror polynucleotides, genes and variants and mutants thereof. “Ror molecule” and “Ror” refer to both Ror1 and Ror2 molecules.

“Target Ror molecule” refers to a Ror molecule whose activity is modulated by the agent of the present invention. The target Ror molecule can be Ror polypeptide, homologues, derivatives or fragments or variants or mutants thereof. Ror molecule of interest can also be nucleic acid (oligonucleotide or polynucleotide of RNA or DNA). For example, if transcripts of genes are the interest of an experiment, the target Ror molecules would be the transcripts. It is to be understood that the term target Ror molecule refers to both full-length molecules and to fragments, variants, and mutants thereof, such as an epitope of a protein. The target Ror molecule may be either Ror1 molecule or Ror2 molecule or both.

The term “Wnt” refers to a family of conserved, cysteine-rich, secreted glycoproteins that are involved in critical aspects of early embryonic development. Wnt genes are also implicated in cancer. “Wnt” as used herein specifically includes Wnt genes of all human and non-human animal species, including, but not limited to, mammals, such as human, mouse, rat and other rodents, etc. The term “Wnt” includes native human Wnt genes and encoded polypeptides, including human Wnt-1 (previously called int-1) (van Ooyen et al., EMBO J, 4, 2905-9, (1985)), Wnt-3 (Roelink et al., Genomics, 17, 790-792, (1993); Huguet et al., Cancer Res., 54, 2615-2521, (1994)), and their variants, in particular amino acid sequence variants.

The “Wnt signaling pathway” refers to any pathway modulated by Wnt proteins such as Wnt-1, Wnt-2, Wnt-3, and the like.

The “canonical Wnt signaling pathway” refers to activation by Wnt proteins of the Disheveled protein which in turn inhibits glycogen synthetase kinase-3 from phosphorylating β-catenin. Phosphorylated β-catenin is rapidly degraded following ubiquitination. However, the unphosphorylated β-catenin accumulates and translocates to the nucleus where it acts as a cofactor of the T-cell factor transcription activator complex.

The term “secreted frizzled related protein” or “SFRP” relates to a secreted receptor of the Wnt signaling pathway.

The term “nucleic acid molecule” refers to the phosphate ester form of ribonucleotides (RNA molecules) or deoxyribonucleotides (DNA molecules), or any phosphodiester analogs, in either single-stranded form, or a double-stranded helix. Double-stranded DNA-DNA, DNA-RNA and RNA-RNA helices are possible. The term nucleic acid molecule, and in particular DNA or RNA molecule, refers to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described according to the normal convention of giving only the sequence in the 5′ to 3′ direction along the nontranscribed strand of DNA (i.e., the strand having a sequence homologous to the mRNA).

A “recombinant nucleic acid molecule” is a nucleic acid molecule that has undergone a molecular biological manipulation, i.e., non-naturally occurring nucleic acid molecule. Furthermore, the term “recombinant DNA molecule” refers to a nucleic acid sequence which is not naturally occurring, or can be made by the artificial combination of two otherwise separated segments of sequence, i.e., by ligating together pieces of DNA that are not normally continuous. By “recombinantly produced” is meant artificial combination often accomplished by either chemical synthesis means, or by the artificial manipulation of isolated segments of nucleic acids, e.g., by genetic engineering techniques using restriction enzymes, ligases, and similar recombinant techniques as described by, for example, Sambrook et al., Molecular Cloning, second edition, Cold Spring Harbor Laboratory, Plainview, N.Y.; (1989), or Ausubel et al., Current Protocols in Molecular Biology, Current Protocols (1989), and DNA Cloning: A Practical Approach, Volumes I and II (ed. D. N. Glover) IREL Press, Oxford, (1985).

Such is usually done to replace a codon with a redundant codon encoding the same or a conservative amino acid, while typically introducing or removing a sequence recognition site. Alternatively, it may be performed to join together nucleic acid segments of desired functions to generate a single genetic entity comprising a desired combination of functions not found in the common natural forms. Restriction enzyme recognition sites are often the target of such artificial manipulations, but other site specific targets, e.g., promoters, DNA replication sites, regulation sequences, control sequences, or other useful features may be incorporated by design. Examples of recombinant nucleic acid molecule include recombinant vectors, such as cloning or expression vectors which contain DNA sequences encoding phl gene proteins which are in a 5′ to 3′ (sense) orientation or in a 3′ to 5′ (antisense) orientation.

The terms “polynucleotide”, “nucleotide sequence”, “nucleic acid”, “nucleic acid molecule”, “nucleic acid sequence”, “oligonucleotide”, refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonuculeotide may comprise a modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil, or containing carbohydrate, or lipids.

Polynucleotides of the invention may be synthesized by standard methods known in the art, e.g. by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988), etc. A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, Nature, 290, 304-310, (1981), the promoter contained in the 3′ long terminal repeat of Rous sarcoma virus, Yamamoto et al., Cell, 22, 787-797, (1980), the herpes thymidine kinase promoter, Wagner et al., Proc. Natl. Acad. Sci. U.S.A. 78, 1441-1445, (1981), the regulatory sequences of the metallothionein gene Brinster et al., Nature 296, 39-42, (1982), etc. Any type of plasmid, cosmid, yeast artificial chromosome or viral vector can be used to prepare the recombinant DNA construct that can be introduced directly into the tissue site. Alternatively, viral vectors can be used which selectively infect the desired tissue, in which case administration may be accomplished by another route (e.g., systemically).

Ribozymes are RNA molecules possessing the ability to specifically cleave other single-stranded RNA in a manner analogous to DNA restriction endonucleases. Through the modification of nucleotide sequences that encode these RNAs, it is possible to engineer molecules that recognize specific nucleotide sequences in an RNA molecule and cleave it, Cech, J. Amer. Med. Assn., 260, 3030, (1988). A major advantage of this approach is that, because they are sequence-specific, only mRNAs with particular sequences are inactivated.

The polynucleotides may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

“RNA transcript” refers to the product resulting from RNA polymerase-catalyzed transcription of a DNA sequence. When the RNA transcript is a complementary copy of the DNA sequence, it is referred to as the primary transcript or it may be an RNA sequence derived from post-transcriptional processing of the primary transcript and is referred to as the mature RNA. “Messenger RNA (mRNA)” refers to the RNA that is without introns and can be translated into polypeptides by the cell. “cRNA” refers to complementary RNA, transcribed from a recombinant cDNA template. “cDNA” refers to DNA that is complementary to and derived from an mRNA template. The cDNA can be single-stranded or converted to double-stranded form using, for example, the Klenow fragment of DNA polymerase I.

A sequence “complementary” to a portion of an RNA, refers to a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

An “anti-sense” copy of a particular polynucleotide refers to a complementary sequence that is capable of hydrogen bonding to the polynucleotide and can therefor be capable of modulating expression of the polynucleotide. These are DNA, RNA or analogs thereof, including analogs having altered backbones, as described above. The polynucleotide to which the anti-sense copy binds may be in single-stranded form or in double-stranded form. A DNA sequence linked to a promoter in an “anti-sense orientation” may be linked to the promoter such that an RNA molecule complementary to the coding mRNA of the target gene is produced.

The antisense polynucleotide may comprise at least one modified sugar moiety selected from the group including but not limited to arabinose, 2-fluoroarabinose, xylulose, and hexose. In one embodiment, the antisense oligonucleotide may comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The term “sense” refers to sequences of nucleic acids that are in the same orientation as the coding mRNA nucleic acid sequence. A DNA sequence linked to a promoter in a “sense orientation” is linked such that an RNA molecule which contains sequences identical to an mRNA is transcribed. The produced RNA molecule, however, need not be transcribed into a functional protein.

A “sense” strand and an “anti-sense” strand when used in the same context refer to single-stranded polynucleotides that are complementary to each other. They may be opposing strands of a double-stranded polynucleotide, or one strand may be predicted from the other according to generally accepted base-pairing rules. Unless otherwise specified or implied, the assignment of one or the other strand as “sense” or “antisense” is arbitrary.

The terms “nucleic acid” or “nucleic acid sequence”, “nucleic acid molecule”, “nucleic acid fragment” or “polynucleotide” may be used interchangeably with “gene”, “mRNA encoded by a gene” and “cDNA”.

The term “polynucleotide encoding polypeptide” encompasses a polynucleotide that may include only the coding sequence as well as a polynucleotide that may include additional coding or non-coding sequence.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength, Sambrook, J. et al. eds., Molecular Cloning: A Laboratory Manual (2d Ed. 1989) Cold Spring Harbor Laboratory Press, NY. Vols. 1-3 (ISBN 0-87969-309-6). The conditions of temperature and ionic strength determine the “stringency” of the hybridization. For preliminary screening for homologous nucleic acids, low stringency hybridization conditions, corresponding to a Tₘ of 55° C., can be used, e.g., 5×SSC, 0.1% SDS, 0.25% milk, and no formamide; or 30% formamide, 5×SSC, 0.5% SDS. Moderate stringency hybridization conditions correspond to a higher Tₘ, e.g., 40% formamide, with 5× or 6×SSC. High stringency hybridization conditions correspond to the highest Tₘ, e.g., 50% formamide, 5× or 6×SSC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived, Sambrook et al. eds., Molecular Cloning: A Laboratory Manual (2d Ed. 1989) Cold Spring Harbor Laboratory Press, NY. Vols. 1-3. (ISBN 0-87969-309-6), 9.50-9.51). For hybridization with shorter nucleic acids, i.e., oligonucleotides, the position of mismatches becomes more important, and the length of the oligonucleotide determines its specificity, Sambrook et al. eds., Molecular Cloning: A Laboratory Manual (2d Ed. 1989) Cold Spring Harbor Laboratory Press, NY. Vols. 1-3. (ISBN 0-87969-309-6, 11.7-11.8).

The term “complementary” is used to describe the relationship between nucleotide bases that are capable to hybridizing to one another. For example, with respect to DNA, adenosine is complementary to thymine and cytosine is complementary to guanine.

“Identity” or “similarity”, as known in the art, are relationships between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Both identity and similarity can be readily calculated by known methods such as those described in: Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991. Methods commonly employed to determine identity or similarity between sequences include, but are not limited to those disclosed in Carillo, H., and Lipman, D., SIAM J Applied Math., 48: 1073 (1988). Methods to determine identity and similarity are codified in publicly available computer programs. Preferred computer program methods to determine identity and similarity between two sequences include, but are not limited to, GCG program package, Devereux, J., et al., Nucleic Acids Research, 12(1), 387 (1984)), BLASTP, BLASTN, and FASTA Atschul, S. F. et al., J Molec. Biol., 215, 403 (1990)).

“Homologous” refers to the degree of sequence similarity between two polymers (i.e. polypeptide molecules or nucleic acid molecules). The homology percentage figures referred to herein reflect the maximal homology possible between the two polymers, i.e., the percent homology when the two polymers are so aligned as to have the greatest number of matched (homologous) positions.

The term “percent homology” refers to the extent of amino acid sequence identity between polypeptides. The homology between any two polypeptides is a direct function of the total number of matching amino acids at a given position in either sequence, e.g., if half of the total number of amino acids in either of the sequences are the same then the two sequences are said to exhibit 50% homology.

The term “fragment”, “analog”, and “derivative” when referring to the polypeptide of the present invention (e.g. SEQ ID NOs:4, 6, 8, 10, and 12), refers to a polypeptide which may retain essentially the same biological function or activity as such polypeptide. Thus, an analog includes a precursor protein that can be activated by cleavage of the precursor protein portion to produce an active mature polypeptide. The fragment, analog, or derivative of the polypeptide of the present invention (e.g. SEQ ID NOs: 4, 6, 8, 10, and 12) may be one in which one or more of the amino acids are substituted with a conserved or non-conserved amino acid residues and such amino acid residues may or may not be one encoded by the genetic code, or one in which one or more of the amino acid residues includes a substituent group, or one in which the polypeptide is fused with a compound such as polyethylene glycol to increase the half-life of the polypeptide, or one in which additional amino acids are fused to the polypeptide such as a signal peptide or a sequence such as polyhistidine tag which is employed for the purification of the polypeptide or the precursor protein. Such fragments, analogs, or derivatives are deemed to be within the scope of the present invention.

“Conserved” residues of a polynucleotide sequence are those residues that occur unaltered in the same position of two or more related sequences being compared. Residues that are relatively conserved are those that are conserved amongst more related sequences than residues appearing elsewhere in the sequences.

Related polynucleotides are polynucleotides that share a significant proportion of identical residues.

Different polynucleotides “correspond” to each other if one is ultimately derived from another. For example, messenger RNA corresponds to the gene from which it is transcribed. cDNA corresponds to the RNA from which it has been produced, such as by a reverse transcription reaction, or by chemical synthesis of a DNA based upon knowledge of the RNA sequence. cDNA also corresponds to the gene that encodes the RNA. Polynucleotides also “correspond” to each other if they serve a similar function, such as encoding a related polypeptide in different species, strains or variants that are being compared.

An “analog” of a DNA, RNA or a polynucleotide, refers to a molecule resembling naturally occurring polynucleotides in form and/or function (e.g. in the ability to engage in sequence-specific hydrogen bonding to base pairs on a complementary polynucleotide sequence) but which differs from DNA or RNA in, for example, the possession of an unusual or non-natural base or an altered backbone. See for example, Uhlmann et al., Chemical Reviews 90, 543-584, (1990).

A “coding sequence” or a sequence “encoding” an expression product, such as an RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein, or enzyme.

“Codon degeneracy” refers to divergence in the genetic code permitting variation of the polynucleotide sequence without affecting the amino acid sequence of an encoded polypeptide. The skilled artisan is well aware of the “codon-bias” exhibited by a specific host cell to use nucleotide codons to specify a given amino acid. Therefore, when synthesizing a gene for improved expression in a host cell, it is desirable to design the gene such that its frequency of codon usage approaches the frequency of preferred codon usage of the host cell.

A “substantial portion” of an amino acid or nucleotide sequence is a portion comprising enough of the amino acid sequence of a polypeptide or the nucleotide sequence of a gene to putatively identify that polypeptide or gene, either by manual evaluation of the sequence by one skilled in the art, or by computer automated sequence comparison and identification using algorithms such as BLAST (Basic Local Alignment Search Tool; Altschul, S. F., et al., J. Mol. Biol. 215, 403-410, (1993); see also www.ncbi.nlm.nih.gov/BLAST).

Accordingly, a “substantial portion” of a nucleotide sequence comprises enough of the sequence to specifically identify and/or isolate a nucleic acid fragment comprising the sequence. The skilled artisan, having the benefit of the sequences as reported herein, may now use all or a substantial portion of the disclosed sequences for purposes known to those skilled in this art.

“Synthetic genes” can be assembled from oligonucleotide building blocks that are chemically synthesized using procedures known to those skilled in the art. These building blocks are ligated and annealed to form gene segments that are then enzymatically assembled to construct the entire gene. “Chemically synthesized”, as related to a sequence of DNA, means that the component nucleotides were assembled in vitro. Manual chemical synthesis of DNA may be accomplished using well-known procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determining preferred codons can be based on a survey of genes derived from the host cell where sequence information is available.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Gene control sequence” refers to the DNA sequences required to initiate gene transcription plus those required to regulate the rate at which initiation occurs. Thus a gene control sequence may consist of the promoter, where the general transcription factors and the polymerase assemble, plus all the regulatory sequences to which gene regulatory proteins bind to control the rate of these assembly processes at the promoter. For example, the control sequences that are suitable for prokaryotes may include a promoter, optionally an operator sequence, and a ribosome-binding site. Eukaryotic cells may utilize promoters, enhancers, and/or polyadenylation signals.

“Promoter” refers to a nucleotide sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a nucleotide sequence that can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic nucleotide segments. It is understood by those skilled in the art that different promoters may direct the expression of a gene in different tissues or cell types, or at different stages of development, or in response to different environmental conditions.

The “3′ non-coding sequences” refer to nucleotide sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

The “translation leader sequence” refers to a nucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency.

The term “operatively linked” refers to the association of two or more nucleic acid fragments on a single nucleic acid fragment so that the function of one is affected by the other. For example, a promoter is operatively linked with a coding sequence when it is capable of affecting the expression of that coding sequence (i.e., that the coding sequence is under the transcriptional control of the promoter). Coding sequences can be operatively linked to regulatory sequences in sense or antisense orientation. A “promoter operable in bone cells” refer to a promoter that is recognized by the RNA polymerase of the bone cell.

“RNA transcript” refers to the product resulting from RNA polymerase-catalyzed transcription of a DNA sequence. When the RNA transcript is a perfect complementary copy of the DNA sequence, it is referred to as the primary transcript or it may be an RNA sequence derived from posttranscriptional processing of the primary transcript and is referred to as the mature RNA. “Messenger RNA (mRNA)” refers to the RNA that is without introns and that can be translated into polypeptide. “cDNA” refers to a double-stranded DNA that is complementary to and derived from mRNA. “Sense” RNA refers to an RNA transcript that includes the mRNA and so can be translated into a polypeptide by the cell. “Antisense RNA” refers to an RNA transcript that is complementary to all or part of a target primary transcript or mRNA and that blocks the expression of a target gene (see U.S. Pat. No. 5,107,065, incorporated herein by reference). The complementarity of an antisense RNA may be with any part of the specific nucleotide sequence, i.e., at the 5′ non-coding sequence, 3′ non-coding sequence, introns, or the coding sequence. “Functional RNA” refers to sense RNA, antisense RNA, ribozyme RNA, or other RNA that may not be translated but yet has an effect on cellular processes.

The term “siRNA” or “RNAi” refers to small interfering RNAs, that are capable of causing interference and can cause post-transcriptional silencing of specific genes in cells, for example, mammalian cells (including human cells) and in the body, for example, mammalian bodies (including humans). The phenomenon of RNA interference is described and discussed in Bass, Nature, 411, 428-29, (2001); Elbahir et al., Nature, 411, 494-98, (2001); and Fire et al., Nature, 391, 806-11, (1998), where methods of making interfering RNA also are discussed. The siRNAs based upon the sequence disclosed herein can be made by approaches known in the art, including the use of complementary DNA strands or synthetic approaches. Exemplary siRNAs could have up to 29 bps, 25 bps, 22 bps, 21 bps, 20 bps, 15 bps, 10 bps, 5 bps or any integer thereabout or therebetween.

The term “expression” refers to the transcription and stable accumulation of sense (mRNA) or antisense RNA derived from the nucleic acid fragment of the invention. Expression may also refer to translation of mRNA into a polypeptide. “Antisense inhibition” refers to the production of antisense RNA transcripts capable of suppressing the expression of the target protein.

“Overexpression” refers to the production of a gene product in an organism that exceeds levels of production in normal or non-transformed organisms. “Suppression” refers to suppressing the expression of foreign or endogenous genes or RNA transcripts.

“Altered levels” refers to the production of gene product(s) in organisms in amounts or proportions that differ from that of normal or non-transformed organisms. Overexpression of the polypeptide of the present invention may be accomplished by first constructing a chimeric gene or chimeric construct in which the coding region is operatively linked to a promoter capable of directing expression of a gene or construct in the desired tissues at the desired stage of development. For reasons of convenience, the chimeric gene or chimeric construct may comprise promoter sequences and translation leader sequences derived from the same genes. 3′ Non-coding sequences encoding transcription termination signals may also be provided. The instant chimeric gene or chimeric construct may also comprise one or more introns in order to facilitate gene expression. Plasmid vectors comprising the instant chimeric gene or chimeric construct can then be constructed. The choice of plasmid vector is dependent upon the method that will be used to transform host cells. The skilled artisan is well aware of the genetic elements that must be present on the plasmid vector in order to successfully transform, select and propagate host cells containing the chimeric gene or chimeric construct. The skilled artisan will also recognize that different independent transformation events will result in different levels and patterns of expression, Jones et al., EMBO J., 4, 2411-2418, (1985); De Almeida et al., Mol. Gen. Genetics, 218, 78-86, (1989), and thus that multiple events must be screened in order to obtain lines displaying the desired expression level and pattern. Such screening may be accomplished by southern analysis of DNA, northern analysis of mRNA expression, western or immunnocytochemical analysis of protein expression, or phenotypic analysis.

An “expression cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.”

The term “polypeptide” refers to a polymer of amino acids without regard to the length of the polymer; thus, “peptides,” “oligopeptides”, and “proteins” are included within the definition of polypeptide and used interchangeably herein. This term also does not specify or exclude chemical or post-expression modifications of the polypeptides of the invention, although chemical or post-expression modifications of these polypeptides may be included or excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. The natural or other chemical modifications, such as those listed in examples above can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, proteins—structure and molecular properties, 2nd Ed., T. E. Creighton, W. H. Freeman and Company, New York (1993); posttranslational covalent modification of proteins, b. c. Johnson, Ed., Academic Press, New York, pgs. 1-12, 1983; Seifter et al., Meth Enzymol 182: 626-646, 1990; Rattan et al., Ann NY Acad Sci 663: 48-62, 1992). Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring. The term “polypeptide” may also be used interchangeably with the term “protein” or “peptide”.

The term “peptide” refers to any polymer of two or more amino acids, wherein each amino acid is linked to one or two other amino acids via a peptide bond (—CONH—) formed between the NH.sub.2 and the COOH groups of adjacent amino acids. Preferably, the amino acids are naturally occurring amino acids, particularly .alpha.-amino acids of the L-enantiomeric form. However, other amino acids, enantiomeric forms, and amino acid derivatives may be included in a peptide. Peptides include “polypeptides,” which, upon hydrolysis, yield more than two amino acids. Polypeptides may include proteins, which typically comprise 50 or more amino acids.

The terms “variant” or “variants” refer to variations of the nucleic acid or amino acid sequences of Ror molecule. Encompassed within the term “variant(s)” are nucleotide and amino acid substitutions, additions, or deletions of Ror molecules. Also, encompassed within the term “variant(s)” are chemically modified natural and synthetic Ror molecules. For example, variant may refer to polypeptides that differ from a reference polypeptide. Generally, the differences between the polypeptide that differs in amino acid sequence from reference polypeptide, and the reference polypeptide are limited so that the amino acid sequences of the reference and the variant are closely similar overall and, in some regions, identical. A variant and reference polypeptide may differ in amino acid sequence by one or more substitutions, deletions, additions, fusions and truncations that may be conservative or non-conservative and may be present in any combination. For example, variants may be those in which several, for instance from 50 to 30, from 30 to 20, from 20 to 10, from 10 to 5, from 5 to 3, from 3 to 2, from 2 to 1 or 1 amino acids are inserted, substituted, or deleted, in any combination. Additionally, a variant may be a fragment of a polypeptide of the invention that differs from a reference polypeptide sequence by being shorter than the reference sequence, such as by a terminal or internal deletion. A variant of a polypeptide of the invention also includes a polypeptide which retains essentially the same biological function or activity as such polypeptide, e.g., precursor proteins which can be activated by cleavage of the precursor portion to produce an active mature polypeptide. These variants may be allelic variations characterized by differences in the nucleotide sequences of the structural gene coding for the protein, or may involve differential splicing or post-translational modification. Variants also include a related protein having substantially the same biological activity, but obtained from a different species. The skilled artisan can produce variants having single or multiple amino acid substitutions, deletions, additions, or replacements. These variants may include, inter alia: (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more amino acids are deleted from the peptide or protein, or (iii) one in which one or more amino acids are added to the polypeptide or protein, or (iv) one in which one or more of the amino acid residues include a substituent group, or (v) one in which the mature polypeptide is fused with another compound, such as a compound to increase the half-life of the polypeptide (for example, polyethylene glycol), or (vi) one in which the additional amino acids are fused to the mature polypeptide such as a leader or secretory sequence or a sequence which is employed for purification of the mature polypeptide or a precursor protein sequence. A variant of the polypeptide may also be a naturally occurring variant such as a naturally occurring allelic variant, or it may be a variant that is not known to occur naturally. All such variants defined above are deemed to be within the scope of teachings in the art.

The polypeptides and the polynucleotides of the present invention are preferably provided in an isolated form, and may be purified to homogeneity.

The term “isolated” means that the material is removed from its original or native environment (e.g., the natural environment if it is naturally occurring). Therefore, a naturally-occurring polynucleotide or polypeptide present in a living animal is not isolated, but the same polynucleotide or polypeptide, separated by human intervention from some or all of the coexisting materials in the natural system, is isolated. For example, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA and combined with carbohydrate, lipid, protein or other materials. Such polynucleotides could be part of a vector and/or such polynucleotides or polypeptides could be part of a composition, and still be isolated in that such vector or composition is not part of the environment in which it is found in nature. Similarly, the term “substantially purified” refers to a substance, which has been separated or otherwise removed, through human intervention, from the immediate chemical environment in which it occurs in Nature. Substantially purified polypeptides or nucleic acids may be obtained or produced by any of a number of techniques and procedures generally known in the field.

The term “purification” refers to increasing the specific activity or concentration of a particular polypeptide or polypeptides in a sample. In one embodiment, specific activity is expressed as the ratio between the activity of the target polypeptide and the concentration of total polypeptide in the sample. In another embodiment, specific activity is expressed as the ratio between the concentration of the target polypeptide and the concentration of total polypeptide. Purification methods include but are not limited to dialysis, centrifugation, and column chromatography techniques, which are well-known procedures to those of skill in the art. See, e.g., Young et al., 1997, “Production of biopharmaceutical proteins in the milk of transgenic dairy animals,” BioPharm 10(6): 34-38.

The terms “substantially pure” and “isolated” are not intended to exclude mixtures of polynucleotides or polypeptides with substances that are not associated with the polynucleotides or polypeptides in nature.

The terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell (e.g., bacterial cells such as E. coli, yeast cells, mammalian cells, avian cells, amphibian cells, plant cells, fish cells, and insect cells), whether located in vitro or in vivo. For example, host and may include any transformable organisms that are capable of replicating a cells may be located in a transgenic animal. Host cell can be used as a recipient for vectors vector and/or expressing a heterologous nucleic acid encoded by a vector.

General methods for expressing and recovering foreign protein produced by a mammalian cell system are provided by, for example, Etcheverry, “Expression of Engineered Proteins in Mammalian Cell Culture,” in Protein Engineering: Principles and Practice, Cleland et al. (eds.), pages 163 (Wiley-Liss, Inc. 1996). Standard techniques for recovering protein produced by a bacterial system is provided by, for example, Grisshammer et al., “Purification of over-produced proteins from E. coli cells,” in DNA Cloning 2: Expression Systems, 2nd Edition, Glover et al. (eds.), pages 59-92 (Oxford University Press 1995). Transformation of insect cells and production of foreign polypeptides therein is disclosed by Guarino et al., U.S. Pat. No. 5,162,222 and WIPO publication WO94/06463. Methods for isolating recombinant proteins from a baculovirus system are also described by Richardson (ed.), “Baculovirus Expression Protocols” (The Humana Press, Inc. 1995). In one embodiment, the polypeptides of the invention can be expressed using a baculovirus expression system (see, Luckow et al., Bio/Technology, 1988, 6, 47, “Baculovirus Expression Vectors: a Laboratory Manual”, O'Rielly et al. (Eds.), W. H. Freeman and Company, New York, 1992, U.S. Pat. No. 4,879,236, each of which is incorporated herein by reference in its entirety). In addition, the MAXBAC™ complete baculovirus expression system (Invitrogen) can, for example, be used for production in insect cells.

The polypeptides of the present invention can also be isolated by exploitation of particular properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3: 1 (1985)). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (M. Deutscher, (ed.), Meth. Enzymol. 182: 529 (1990)). Within additional embodiments of the invention, a fusion of the polypeptide of interest and an affinity tag (e.g., maltose-binding protein, an immunoglobulin domain) may be constructed to facilitate purification.

Host cells of the invention can be used in methods for the large-scale production of Ror polypeptides wherein the cells are grown in a suitable culture medium and the desired polypeptide products are isolated from the cells, or from the medium in which the cells are grown, by purification methods known in the art, e.g., conventional chromatographic methods including immunoaffinity chromatography, receptor affinity chromatography, hydrophobic interaction chromatography, lectin affinity chromatography, size exclusion filtration, cation or anion exchange chromatography, high pressure liquid chromatography (HPLC), reverse phase HPLC, and the like. Other methods of purification include those methods wherein the desired protein is expressed and purified as a fusion protein having a specific tag, label, or chelating moiety that is recognized by a specific binding partner or agent. The purified protein can be cleaved to yield the desired protein, or can be left as an intact fusion protein. Cleavage of the fusion component may produce a form of the desired protein having additional amino acid residues as a result of the cleavage process.

The term “in Situ:” refers to and includes the terms “in vivo,” “ex vivo,” and “in vitro” as these terms are commonly recognized and understood by persons ordinarily skilled in the art. Furthermore, the phrase “in situ” is employed herein in its broadest connotative and denotative contexts to identify an entity, cell or tissue as found or in place, without regard to its source or origin, its condition or status or its duration or longevity at that location or position.

The term “in vitro” refers to an artificial environment and to reactions or processes that occur within an artificial environment. In vitro environments include, but are not limited to, test tubes and cell cultures. The term “in vivo” refers to the natural environment (e.g., an animal or a cell) and to processes or reaction that occur within a natural environment.

The methods of the present invention may be performed in vitro using cells (cultured cells) and cell lysates, including nuclear extracts. Examples of cells contemplated for identifying agents that modulate bone formation include but are not limited to calvarial cells, osteoblasts, osteoclasts, chondrocytes, and pluripotent precursor cells, such as multipotent bone marrow stromal cells. Specific examples of osteoblast and osteoblast precursor cell lines include MC3T3-E1, C2C12, MG-63 cells, U2OS cells, UMR106 cells, ROS 17/2.8 cells, SaOS-2 cells, and the like that are provided in the catalog from the ATCC (WO 01/19855) as well as HOB cell lines described in Bodine P V, Vernon S K, Komm B S., Endocrinology, 137, 4592-4604, (1996), Bodine P V N, TrailSmith M, Komm B S., J Bone Min Res, 11, 806-819, (1996), Bodine P V, Green J, Harris H A, Bhat R A, Stein G S, Lian J B, Komm B S., J Cell Biochem, 65, 368-387, (1997), Bodine P V, Komm B S., Bone, 25, 535-43 (1999), Bodine P V N, Harris H A, Komm B S., Endocrinology, 140, 2439-2451, (1999), Prince M, Banerjee C, Javed A, Green J, Lian J B, Stein G S, Bodine P V, Komm B S, J Cell Biochem, 80, 424-40, (2001).

The methods of the present invention may also be performed using a cell-free system.

The term “expression system” refers to a host cell and compatible vector under suitable conditions, e.g., for the expression of a protein coded for by foreign DNA carried by the vector and introduced into the host cell. Common expression systems include E. coli host cells and plasmid vectors, insect host cells and Baculovirus vectors, and mammalian host cells and vectors.

“Transformation” refers to the transfer of a nucleic acid fragment into the genome of a host organism, resulting in genetically stable inheritance. Host organisms containing the transformed nucleic acid fragments are referred to as “transgenic” organisms.

“Clone” refers to a population of cells derived from a single cell or common ancestor by mitosis. A “cell line” refers to a clone of a primary cell that is capable of stable growth in vitro for several generations.

The term “differentiate” refers to having a different character or function from the original type of tissues or cells. Thus, “differentiation” is the process or act of differentiating.

The term “osteoblast differentiation” refers to the process in which a cell develops specialized functions during maturation into an osteoblast cell. Osteoblast differentiation may include pre-osteoblast, early and mature osteoblast, pre-osteocyte and mature osteocyte stages (Bodine et al, Vitamins and Hormones 65, 101-151 (2002), Stein et al. Endocrine Reviews 14, 424-442 (1993), and Lian et al. Vitamins and Hormones 55, 443-509 (1999)).

The term “proliferation” refers to the growth and production of similar cells.

The term “phenotype” refers to the observable character of a cell or an organism. Such observable character can involve the physical appearance, as well as a level of particular physiological compositions present in the cell or organism. Ostoeblastic phenotype includes expression of several marker proteins such as bone-specific transcription factor Cbfa1; type I collagen; alkaline phosphatase, osteocalcin; and bone sialoprotein.

The term “gene-inducible system” refers to the use of ligands to regulate gene expression. Several regulatory systems have been developed that utilize small molecules to induce gene expression (reviewed in Clackson T., Curr Opin Chem Biol, 1, 210-218, (1997); Lewandoski M., Nat Rev Genet., 2, 743-755, (2001). A gene-inducible system is a molecular tool which allows for low to undetectable basal expression of a target gene when the system is not activated and increased expression levels of the target gene when the system is activated.

“Mature” protein refers to a post-translationally processed polypeptide, i.e., one from which any pre- or pro-peptides present in the primary translation product have been removed. “Precursor” protein refers to the primary product of translation of mRNA, i.e., with pre- and pro-peptides still present. Pre- and pro-peptides include but are not limited to intracellular localization signals.

As used herein, the term “binding partner” or “interacting proteins” refer to a molecule capable of binding another molecule with specificity, as for example, an antigen and an antigen-specific antibody or an enzyme and its inhibitor. Binding partners may include, for example, biotin and avidin or streptavidin, IgG and protein A, receptor-ligand couples, protein-protein interaction, and complementary polynucleotide strands. The term “binding partner” may also refer to polypeptides, lipids, small molecules, or nucleic acids that bind to kinases in cells. A change in the interaction between a kinase and, a binding partner can manifest itself as an increased or decreased probability that the interaction forms, or an increased or decreased concentration of kinase-binding partner complex. For example, Ror1 or Ror 2 protein may bind with another protein or polypeptide and form a complex that may result in modulating Ror1 or Ro2 activity.

The term “signal transduction pathway” refers to the molecules that propagate an extracellular signal through the cell membrane to become an intracellular signal. This signal can then stimulate a cellular response. The polypeptide molecules involved in signal transduction processes may be receptor and non-receptor protein tyrosine kinases.

“Receptor” refers to a molecular structure within a cell or on the surface of the cell that is generally characterized by the selective binding of a specific substance. Exemplary receptors include cell-surface receptors for peptide hormones, neurotransmitters, antigens, complement fragments and immunoglobulins as well as cytoplasmic receptors for steroid hormones.

The term “modulate” refers to the suppression, enhancement, or induction of a function. For example, “modulation” or “regulation” of gene expression refers to a change in the activity of a gene. Modulation of expression can include, but is not limited to, gene activation and gene repression. “Modulate” or “regulate” also refers to methods, conditions, or agents which increase or decrease the biological activity of a protein, enzyme, inhibitor, signal transducer, receptor, transcription activator, co-factor, and the like. This change in activity can be an increase or decrease of mRNA translation, DNA transcription, and/or mRNA or protein degradation, which may in turn correspond to an increase or decrease in biological activity. Such enhancement or inhibition may be contingent upon occurrence of a specific event, such as activation of a signal transduction pathway and/or may be manifest only in particular cell types.

“Modulated activity” refers to any activity, condition, disease or phenotype that is modulated by a biologically active form of a protein. Modulation may be affected by affecting the concentration of biologically active protein, e.g., by regulating expression or degradation, or by direct agonistic or antagonistic effect as, for example, through inhibition, activation, binding, or release of substrate, modification either chemically or structurally, or by direct or indirect interaction which may involve additional factors.

“Modulator” refers to any agent that alters the expression of a specific activity, such as bone formation or Ror molecule expression. For example, an agent that modulates bone formation alters or changes (increases or decreases) bone formation. The modulator is intended to comprise any compound, e.g., antibody, small molecule, peptide, oligopeptide, polypeptide, or protein.

The term “small molecule” refers to a synthetic or naturally occurring chemical compound, for instance a peptide or oligonucleotide that may optionally be derivatized, natural product or any other low molecular weight (typically less than about 5 kDalton) organic, bioinorganic or inorganic compound, of either natural or synthetic origin. Such small molecules may be a therapeutically deliverable substance or may be further derivatized to facilitate delivery.

As used herein, the term “inducer” refers to any agent that induces, enhances, promotes or increases a specific activity, such as bone formation, or Ror molecule expression.

As used herein the term “inhibitor” or “repressor” refers to any agent that inhibits, suppresses, represses, or decreases a specific activity, such as bone formation, or Ror molecule expression.

As used herein, the term “agent” or “test agent” refers to any compound or molecule that is to be tested.

Examples of agents of the present invention include but are not limited to peptides, small molecules, and antibodies. Agents can be randomly selected or rationally selected or designed. As used herein, an agent is said to be “randomly selected” when the agent is chosen randomly without considering the specific interaction between the agent and the target compound or site. As used herein, an agent is said to be “rationally selected or designed”, when the agent is chosen on a non-random basis which takes into account the specific interaction between the agent and the target compound or site and/or the conformation in connection with the agent's action.

As used herein, the term “antibody” refers to an immnuoglobulin molecule or immunologically active portion thereof, i.e. antigen-binding portion. Examples of immunologically active portion of immnuoglobulin molecules include F(ab), Fv, and F(ab′) fragments which can be generated by cleaving the antibody with an enzyme such as pepsin.

As used herein, the terms “treatment”, “treating”, and “therapy” refer to therapeutic treatment and prophylactic, or preventative manipulations, or manipulations which stimulate bone cell differentiation or bone formation, postpone the development of bone disorder symptoms, and/or reduce the severity of bone disorders and/or such symptoms that will or are expected to develop from a bone disorder. The terms further include ameliorating existing bone disorder symptoms, preventing additional symptoms, ameliorating or preventing the underlying metabolic causes of symptoms, preventing or reversing metabolic causes of symptoms, or, preventing or promoting bone growth. Thus, the terms denote that a beneficial result has been conferred on a subject with a bone disorder, or with the potential to develop such disorder. Furthermore, the term “treatment” is defined as the application or administration of an agent (e.g., therapeutic agent or a therapeutic composition) to a subject, or an isolated tissue or cell line from a subject, Who may have a disease, a symptom of disease or a predisposition toward a disease, with the purpose to cure, heal, alleviate, relieve, alter, remedy, ameliorate, improve or affect the disease, the symptoms of disease or the predisposition toward disease. As used herein, a “therapeutic agent” refers to any substance or combination of substances that assists in the treatment of a disease, e.g., modulates bone forming activity or induces new bone formation. Accordingly, a therapeutic agent includes, but is not limited to, small molecules, peptides, antibodies, ribozymes and antisense oligonucleotides.

Therapeutic agent or therapeutic compositions may also include a compound in a pharmaceutically acceptable form that prevents and/or reduces the symptoms of a particular disease. For example a therapeutic composition may be a pharmaceutical composition that prevents and/or reduces the symptoms of a bone related disorder. It is contemplated that the therapeutic composition of the present invention will be provided in any suitable form. The form of the therapeutic composition will depend on a number of factors, including the mode of administration. The therapeutic composition may contain diluents, adjuvants and excipients, among other ingredients.

The bone strength may be determined by bone density (grs of mineral/cm³ of volume) and bone quality (mineralization, bone architecture, bone turnover, micro fractures). As a measure for bone strength, Bone Mineral Density (BMD) is usually used. For example, a bone can be declared osteoporotic if its BMD is exceeds 2.5 standard deviations below the mean of BMD of young white adult women (World Health Organization, 1994, Assessment of Fracture Risk and it's Application to Screening for Postmenopausal Osteoporosis. Technical Report Series 843. Geneva: World health Organization).

“Bone tissue” refers to calcified tissues (e.g., calvariae, tibiae, femurs, vertebrae, teeth), bone trabeculae, the bone marrow cavity, which is the cavity other than the bone trabeculae, the cortical bone, which covers the outer peripheries of the bone trabeculae and the bone marrow cavity, and the like. Bone tissue also refers to bone cells that are generally located within a matrix of mineralized collagen; blood vessels that provide nutrition for the bone cells; bone marrow aspirates: joint fluids: bone cells that are derived from bone tissues; and may include fatty bone marrow. Bone tissue includes bone products such as whole bones, sections of whole bone, bone chips, bone powder, bone tissue biopsy, collagen preparations, or mixtures thereof. For the purposes of the present invention, the term “bone tissue” is used to encompass all of the aforementioned bone tissues and products, whether human or animal, unless stated otherwise.

As used herein, “bone-related activity” includes bone-forming activity and bone-resorbing activity.

Bone-forming activity can be induced by increasing osteoblastic activity, osteoblastic differentiation from osteoprogenitor cells, and osteoblastic profilteration, by decreasing osteoblast apoptosis and by any combination thereof. In addition, bone-resorbing activity can be suppressed by decreasing osteoclast activity, osteoclast differentiation and proliferation, by increasing osteoclast apoptosis and by any combination thereof. Bone-forming activity can be induced in various bone tissues or cells.

As used herein, the phrase “modulating bone formation” refers to increase or decrease in bone formation. “Increased bone formation” is meant the recruitment of osteoblasts or osteoblast precursors to a bone site, which results in differentiation of the cells inot mature osteoblasts and their secretion of collagenous matrix which minarralizes int bone matter and increases bone mass at the site. The term also encompasses the increased production and secretion of collagenous matrix by mature osteoblasts. Increased bone formation can be determined via one or more of a decrease in fracture rate, an increase in areal bone density, an increase in volumetric mineral bone density, an increase in trabecular connectivity, an increase in trabecular density, an increase in cortical density or thickness, an increase in bone diameter, and an increase in inorganic bone content. Increased bone formation may result from increased attachment, proliferation, survival and/or differentiation of bone cells, e.g., osteoblasts, and subsequent bone mineralization.

“Bone-related disorders” include disorders of bone formation and bone resorption. These diseases and conditions include, but are not limited to, rickets, osteomalacia, osteopenia, osteosclerosis, renal osteodystrophy, osteoporosis (including senile and postmenopausal osteoporosis), Paget's disease, bone metastases, hypercalcaemia, hyperparathyroidism, osteopetrosis, periodontitis, and the abnormal changes in bone metabolism which may accompany rheumatoid arthritis and osteoarthritis. Some of these diseases are characterized by insufficient bone formation or bone loss, while others involve an abnormal thickening or hardening of bone tissue. Examples of diseases that would benefit from inhibiting abnormal thickening of the bone include but are not limited to osteopetrosis and osteosclerosis.

“Bone-related agents” refer to agents that influence bone formation or bone resorption. “Bone-related agents” may induce anabolic or catabolic effect, may inhibit bone resorption and result in increased bone mineral density, may increase bone formation, or may maintain the balance between bone formation and bone resorption.

The terms “compound” or “agent” are used interchangeably herein to refer to a compound or compounds or composition of matter which, when administered to a subject (human or animal) induces a desired pharmacologic and/or physiologic effect by local and/or systemic action.

The term “subject” refers to any mammal, including a human, or non-human subject. Non-human subjects can include experimental, test, agricultural, entertainment or companion animals.

The term “biological sample” is broadly defined to include any cell, tissue, biological fluid, organ, multi-cellular organism, and the like. A biological sample may be derived, for example, from cells or tissue cultures in vitro. Alternatively, a biological sample may be derived from a living organism or from a population of single-cell organisms. A biological sample may be a live tissue such as live bone. The term “biological sample” is also intended to include samples such as cells, tissues or biological fluids isolated from a subject, as well as samples present within a subject. That is, the detection method of the invention can be used to detect Ror mRNA, protein, genomic DNA, or activity in a biological sample in vitro as well as in vivo. For example, in vitro techniques for detection of Ror mRNA include TaqMan analysis, northern hybridization, and in situ hybridization. In vitro techniques for detection of Ror protein include enzyme-linked immunosorbent assays (ELISAs), western blots, immunoprecipitations and immunofluorescence. In vitro techniques for detection of Ror genomic DNA include southern hybridizations.

A “test sample” refers to a biological sample from a subject of interest.

“Body fluid” refers to any body fluid including, without limitation, serum, plasma, lymph fluid, synovial fluid, follicular fluid, seminal fluid, amniotic fluid, milk, whole blood, sweat, urine, cerebro-spinal fluid, saliva, sputum, tears, perspiration, mucus, tissue culture medium, tissue extracts, and cellular extracts. It may also apply to fractions and dilutions of body fluids. The source of a body fluid can be a human body, an animal body, an experimental animal, a plant, or other organism.

### DESCRIPTION OF SPECIFIC EMBODIMENTS OF THE INVENTION

Method of Using the Agents that Modulate the Expression or Activity of Ror Molecule as Modulators of Bone-Related Activity: An agent that modulates the expression or activity of Ror molecule is useful for modulating bone-related activity. There are many diseases and conditions characterized by the need to modulate bone related activity, e.g. enhance bone formation. The most obvious is the case of bone fractures, where it would be desirable to stimulate bone growth and to hasten and complete bone repair. For example, agents that enhance bone formation may be potentially useful in facial reconstruction procedures. Other bone deficit conditions include, but are not limited to, bone segmental defects, periodontal disease, metastatic bone disease, osteolytic bone disease and conditions where connective tissue repair would be beneficial, such as healing or regeneration of cartilage defects or injury. Also of great significance is the chronic condition of osteoporosis, including age-related osteoporosis and osteoporosis associated with post-menopausal hormone status. Other conditions characterized by the need for bone growth include primary and secondary hyperparathyroidism, diabetes-related osteoporosis, disuse osteoporosis, and glucocorticoid-related osteoporosis.

Agents for use in the methods of the invention can be incorporated into pharmaceutical compositions suitable for administration. As used herein, the term “agent” includes, but is not limited to, Ror nucleic acid molecules, fragments of Ror polypeptide, and anti-Ror antibodies, as well as identified compounds (e.g., small, orally active, organic molecules) that modulate Ror molecule expression, synthesis, and/or activity. Such compositions typically comprise the compound, nucleic acid molecule, protein, antibody as well as vectors and host cells that express such nucleic acid molecules, and a pharmaceutically acceptable carrier. The compositions of the present invention may contain one or more agent in combination with one or more agents known to modulate bone-related activity. For example, an agent that induces Ror expression may be combined with agents that inhibit bone resorption like estrogens, bisphosphonates, or tissue selective estrogens (i.e, selective estrogen receptor modulators or SERMs).

One or more agent is used at a therapeutically effective dose. A therapeutically effective dose refers to that amount of the agent that is sufficient to show a benefit (e.g., a reduction in a symptom associated with the disorder, disease, or condition being treated). When applied to an individual ingredient, administered alone, the term refers to that ingredient alone. When applied to a combination, the term refers to combined amounts of the ingredients that result in the benefit, whether administered in combination, serially, or simultaneously. For example, an effective amount for therapeutic uses is the amount of the composition comprising an agent that provides a clinically significant increase in healing rates in fracture repair; reversal of bone loss and prevention of fractures in osteoporosis; reversal of cartilage defects or disorders; prevention or delay of onset of osteoporosis; stimulation and/or inhibition of bone formation in fracture non-unions and distraction osteogenesis; increase and/or decrease in bone growth into prosthetic devices; repair of dental defects; and the like. Such effective amounts will be determined using routine optimization techniques and are dependent on the particular condition to be treated, the condition of the patient, the route of administration, the formulation, and the judgment of the practitioner and other factors evident to those skilled in the art. The dosage required for the compounds of the invention (for example, in osteoporosis where an increase in bone formation is desired) is the dosage that ensures a statistically significant difference in bone mass between treatment and control groups. This difference in bone mass may be seen, for example, as a 5-20% or more increase in bone mass in the treatment group. Other measurements of clinically significant increases in healing may include, for example, tests for breaking strength and tension, breaking strength and torsion, 4-point bending, increased connectivity in bone biopsies and other biomechanical tests well known to those skilled in the art. General guidance for treatment regimens may be obtained from experiments carried out in animal models of the disease of interest.

Toxicity and therapeutic efficacy of agents may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Agents or compounds that exhibit large therapeutic indices are preferred. The data obtained from cell culture assays and animal studies may be used in formulating a range of dosage for use in humans. The dosage of such agents or compounds may be within a range of circulating concentrations that include the ED₅₀ with little or no toxicity. The dosage may vary within this range depending upon the dosage form employed and the route of administration utilized.

For any agent used in the method of the invention, the therapeutically effective dose may be estimated initially from cell culture assays. For example, a dose can be formulated in animal models to achieve a circulating plasma concentration range that includes the ED₅₀ as determined in cell culture (i.e., the concentration of the test compound which achieves a half-maximal disruption of the protein complex, or a half-maximal inhibition of the cellular level and/or activity of a complex component). Such information can be used to more accurately determine useful doses in humans. Levels in plasma may be measured, for example, by HPLC. The dosage can be chosen by the individual physician in view of the patient's condition. The attending physician would know how to and when to terminate, interrupt, or adjust administration. Conversely, the attending physician would also know to adjust treatment to higher levels if the clinical response were not adequate (precluding toxicity). The magnitude of an administered dose in the management of the disorder of interest will vary with the severity of the condition to be treated. The severity of the condition may, for example, be evaluated, in part, by standard prognostic evaluation methods. Further, the dose and perhaps dose frequency, will also vary according to the age, body weight, and response of the individual patient. A program comparable to that discussed above may be employed in veterinary medicine.

Determination of the proper dosage for a particular situation is within the skill of the art. Generally, treatment is initiated with smaller dosages that are less than the optimum dose of the compound. Thereafter, the dosage may be increased by small increments until the optimum effect under the circumstances is reached. For example, the total daily dosage may be divided and administered in portions during the day, if desired.

Depending on the specific conditions being treated, agents may be formulated and administered systemically or locally. A pharmaceutical composition of the invention is formulated to be compatible with its intended route of administration. Techniques for formulation and administration may be found in Remington's Pharmaceutical Sciences, 18th ed., Mack Publishing Co., Easton, Pa. (1990). Suitable routes may include oral, rectal, vaginal, transdermal, transmucosal, or intestinal administration; parenteral delivery, including intramuscular, subcutaneous and intramedullary injections; as well as intrathecal, direct intraventricular, intravenous, intraperitoneal, intranasal, or intraocular injections, just to name a few. Some methods of delivery that may be used include but are not limited to encapsulation in liposomes, transduction by retroviral vectors, localization to nuclear compartment utilizing nuclear targeting site found on most nuclear proteins, transfection of cells ex vivo with subsequent reimplantation or administration of the transfected cells, and a DNA transporter system.

When the compositions are used pharmaceutically, they are combined with a “pharmaceutically acceptable carrier” for diagnostic and therapeutic use. The formulation of such compositions is well known to persons skilled in this field. Pharmaceutical compositions of the invention may comprise one or more additional agents and, preferably, include a pharmaceutically acceptable carrier.

Suitable pharmaceutically acceptable carriers and/or diluents include any and all conventional solvents, dispersion media, fillers, solid carriers, aqueous solutions, coatings, antibacterial and antifungal agents, isotonic and absorption delaying agents, and the like. The term “pharmaceutically acceptable carrier” refers to a carrier that does not cause an allergic reaction or other untoward effect in patients to whom it is administered. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of one or more of the agents of the composition. The use of such media and agents for pharmaceutically substances is well known in the art.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. Pharmaceutical compositions suitable for injections include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars or polyalcohols such as mannitol, sorbitol, sodium chloride in the composition. Prolonged absorption of the injectable compositions can be brought about by including in the composition an agent which delays absorption, for example, aluminum monostearate and gelatin.

Further, the agents for treating diseases and conditions identified by the present invention may also be co-administered with other therapeutic agents that are selected for their particular usefulness against the condition that is being treated. For example, the agents may be combined with estrogens or estrogen-related compounds or other bone resorption inhibitors. Estrogen compounds include but are not limited to conjugated estrogens, estradiol, and analogs thereof. Other bone-related therapeutic compounds include, but are not limited to, bisphosphonates and related compounds (such as those set forth in U.S. Pat. No. 5,312,814), calcium supplements (Prince, R. L. et al., N. Engl. J. Med. 325, 1189, (1991), vitamin D supplements (Chapuy M. C. et al., N. Engl. J. Med. 327, 1637, (1992), sodium fluoride (Riggs, B. L. et al., N. Engl. J. Med., 327, 620, (1992), androgen (Nagent de Deuxchaisnes, C., in Osteoporosis, a Multi-Disciplinary Problem, Royal Society of Medicine International Congress and Symposium Series No. 55, Academic Press, London, p. 291, (1983), and calcitonin (Christiansen, C., Bone 13 (Suppl. 1):S35, (1992).

Ror Molecules as Pharmaceutical Drug Targets: The present invention validates Ror molecules as osteoporotic drug targets by in vitro and in vivo approaches. For example, siRNA molecules may be generated that would specifically disrupt Ror expression in mammalian cells. These siRNAs can be over-expressed in early osteoblasts with relatively high levels of Ror mRNA and the effects of Ror disruption on cell differentiation and/or survival can be monitored. Gene chip analysis may also be performed to identify Ror-dependent genes (U.S. Pat. No. 5,795,716, U.S. Pat. No. 5,974,164). Based on Ror2 expression patterns and its relationship to SFRP-1 and Wnt signaling, Ror2 down-regulation can speed up the osteoblast differentiation and can also promote apoptosis. A complementary approach involves overexpression of Ror in pre-osteocytic cells that have no detectable levels of Ror2 mRNA and low levels of Ror1 mRNA and monitoring the differentiation state of these cells. Following in vitro validation, in vivo validation of Ror as an osteoporotic target may be performed by generating transgenic mice conditionally overexpressing Ror in a tissue- and/or time-dependent manner. Mice with Ror expression conditionally disrupted at different times during development can also be generated.

Method of Identifying Agents that Modulate Bone-Related Activity and/or Wnt Signaling Pathway: The present invention provides a method for identifying agents that modulate bone-related activity and/or Wnt signaling comprising administering a test agent and monitoring the expression or activity of Ror molecule to determine whether the agent modulates bone-related activity, wherein an increase or decrease in the expression or activity of Ror molecule indicates that the agent modulates bone-related activity and/or Wnt signaling.

Methods for determining whether an agent alters the expression or activity of Ror molecule include performing analyses and assays well known to the skilled artisan. Examples include but are not limited to histochemical analysis, northern blot analysis, TaqMan analysis, western blot analysis, ELISA, and functional analyses including, for example, measurements of the extent of Ror phosphorylation (higher state of phosphorylation reflecting higher activity). Other methods contemplated by the present invention for identifying test agents that modulate Ror expression and activity include PCR analysis and reporter gene systems. The reporter gene can encode luciferase and can be under the control of, for example, a promoter that is activated by Wnt-3 signaling. Since Ror1 and Ror2 inhibit Wnt-3 activity, the expression of the reporter reflects changes in Ror molecule expression and/or activity.

The methods of the present invention may be modified or performed in any available format, including high throughput assays. High throughput assays are useful for screening a large number of compounds in a given period of time. In one embodiment, assays can be formed using nucleic acids, wherein nucleic acids are placed on a DNA chip. In another embodiment, assays using cell-based screening are performed. U.S. Pat. No. 6,103,479 discloses miniature cell array methods and apparatus for cell-based screening. Methods have been described for making uniform micro-patterned arrays of cells for other applications, for example photochemical resist-photolithograpy (Mrksich and Whitesides, Ann. Rev. Biophys. Biomol. Struct., 25, 55-78, (1996)). U.S. Pat. No. 6,096,509 provides an apparatus and method for real-time measurement of a cellular response to a test compound on a flowing suspension of cells, in which a homogeneous suspension of each member of a series of cell types is combined with a test compound at a specific concentration, directed through a detection zone, and a cellular response of the living cells is measured in real time as the cells in the test mixture are flowing through the detection zone. The patent discloses the use of the apparatus in automated screening of libraries of compounds. The methods disclosed in the U.S. Patents can be modified to determine whether test agents modulate the expression or activity of Ror molecule using cells such as osteoblastic cells (HOB, U2OS, SaOS-2 and others) or non-osteoblastic cells (COS-7 and others).

Methods of Identifying Genes or Proteins that Modulate Bone-related Activity and/or Wnt Signaling Pathway: The present invention also contemplates methods of identifying genes or proteins that may be regulated by Ror expression and therefore may participate in bone-related activity. For example, such genes or proteins may be identified by overexpressing Ror molecules and monitoring the changes in gene expression profiles. In addition, the effects of Ror down-regulation (by means of anti-sense RNA or siRNA) on gene expression profiles may be investigated. Furthermore, genes or proteins that are regulated by Ror expression may have effects on Wnt signaling pathway.

In higher organisms, the expression of certain genes in a cell determines the life processes, e.g. development and differentiation, homeostasis, response to various agents, cell cycle regulation, aging, apoptosis, etc., to be carried out by the cell. Alterations in gene expression change the course of development of a normal cell. Therefore, methods for analyzing gene expression are critical to basic molecular biological research. Identification of differentially-expressed genes may provide a key to diagnosis, prognosis, and treatment of a variety of diseases or condition states in animals, including humans. Additionally, these methods can be used to identify differentially-expressed sequences due to changes in gene expression level associated with predisposition to disease or conditions, e.g., associated with bone-related activity.

For example, differential gene expression assays can be performed in a particular cell and the expression of genes in different cells can be compared and any discrepancies in expression are identified, where the presence of discrepancies indicates a difference in the classes of genes expressed in the cells being compared.

As used herein, the term “differential gene expression” refers to both quantitative as well as qualitative differences in the genes' temporal and/or tissue expression patterns. Thus, a differentially expressed gene may qualitatively have its expression activated or completely inactivated in normal versus abnormal bone formation state or abnormal body weight state, or under control versus experimental conditions. Such a qualitatively regulated gene will be detectable in either control subjects or subjects with a disease, but not in both. Alternatively, such a qualitatively regulated gene will be detectable in either control or experimental subjects, but not in both. The term “detectable” refers to expression pattern, e.g., an RNA expression pattern that is detectable via the standard techniques of differential display, RT-PCR and/or northern analyses, which are well known to those of skill in the art.

Various models of normal and abnormal animals or cell lines can be used to identify differentially expressed genes that are modulated or regulated by Ror. For example, cell lines derived from normal individuals and individuals with diseases including, but not limited to, osteoporosis, rickets, osteomalacia, osteopenia, osteosclerosis, hyperparathyroidism, osteopetrosis, periodontitis, renal osteodystrophy, Paget's disease, bone metastases, hypercalcaemia, obesity, anorexia, cachexia, and nonshivering and shivering thermogenesis, are utilized to study differential gene expression associated with Ror expression.

In order to identify differentially expressed genes, RNA, either total or mRNA, may be isolated from the tissues of the animals or from the cells described above. RNA samples may be obtained from tissues of test subjects and from corresponding tissues of control subjects. Any RNA isolation technique which does not select against the isolation of mRNA may be utilized for the purification of such RNA samples (e.g., Ausubel, F. M. et al, eds., (1987-1993), Current Protocols in Molecular Biology, John Wiley & Sons, Inc. New York). Additionally, large numbers of tissue samples may readily be processed using techniques well known to those of skill in the art (e.g. U.S. Pat. No. 4,843,155).

Transcripts within the collected RNA samples that represent RNA produced by differentially expressed genes may be identified by utilizing methods well known in the art. For example, gene chip analysis (U.S. Pat. No. 5,795,716, U.S. Pat. No. 5,974,164), cDNA microarray analysis, see, for example, Ono K, Tanaka T, Tsunoda T, Kitahara O, Kihara C, Okamoto A, Ochiaia K, Takagi T, Nakamura Y., Cancer Res 60, 5007-5011, (2000), subtractive hybridization (Hedrick et al., Nature 308, 149-153, (1984); Lee et al., Proc. Natl. Acad. Sci. USA, 88, 2825, (1984), or differential display (Liang et al., Science 257, 967-971 (1992), U.S. Pat. No. 5,262,311) may be utilized to identify nucleic acid sequences derived from genes that are differentially expressed.

Once potentially differentially expressed gene sequences have been identified via techniques described above, the differential expression of such putatively differentially expressed genes can be further characterized using well known techniques in the art such as TaqMan analysis, northern analysis, or quantitative RT-PCR.

Generating Host Cells: Ror nucleotide sequences may be over-expressed in different cell lines to identify their role in various cell functions by the techniques known in the art. For example, Ror can be over-expressed in osteoblastic cell lines and its role in differentiation may then be monitored by the standard techniques. Furthermore, Ror can be used in a gene-inducible system in a transient transfection assays to determine whether an agent has effects on bone formation.

Host cells can be engineered with the vectors containing Ror nucleotide sequences. The host organism (recombinant host cell) may be any eukaryotic or prokaryotic cell, or multicellular organism. They may be derived from mammals, yeast, fungi, or viruses. Suitable host cells may include but are not limited to mammalian cells, e.g., non-osteoblastic cells (monkey kidney COS-7, human kidney 293, hamster ovary CHO, human liver HepG2, human cervical HeLa, or mouse fibroblast NIH3T3) or osteoblastic cells (primary osteoblasts, human osteoblastic cells such as TE-85, U2OS, SaOS-2 or HOB, rat osteoblastic cells such as UMR 106 or ROS 17/2.8, or mouse osteoblastic cells such as MC3T3. Furthermore, various strains of E. coli (e.g., DH5alpha, BL21, DH10B), yeast cells (e.g., Schizasaccharomyces, Saccharomyces cerevisice, Pichia Pastoris, Pichia methanolica) and insect cells (SF9, SF21, Spodoptera frugiperda, S2 Schneider Cells, High Five Cells from Trichoplusia ni egg) may be used as host cells for molecular biological manipulation.

The vectors may be cloning vectors or expression vectors such as in the form of a plasmid, a cosmid, or a phage or any other vector that is replicable and viable in the host cell. The engineered host cells can be cultured in conventional nutrient media modified as appropriate for activating promoters, selecting transformants or amplifying the polynucleotide of the present invention. The culture conditions such as pH, temperature, and the like, are those suitable for use with the host cell selected for expression of the polynucleotide as known to the ordinarily skilled in the art.

Plasmids generally are designated herein by a lower case “p” preceded and/or followed by capital letters and/or numbers, in accordance with standard naming conventions that are familiar to those of skill in the art. The plasmids herein are either commercially available, publicly available on unrestricted bases, or can be constructed from available plasmids by routine application of well-known, published procedures. Additionally, many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

The appropriate DNA sequence may be inserted into the vector by a variety of the procedures known in the art.

The DNA sequence in the expression vector may be operatively linked to an appropriate expression control element(s) to direct mRNA synthesis. The expression control elements are known in the art and include, but are not limited to, inducible promoters, constitutive promoters, secretion signals, and other regulatory elements. Preferably, the inducible promoter is readily controlled, such as being responsive to a nutrient in the host cell's medium. Appropriate non-native mammalian promoters might include cytomegalovirus (CMV), Rous sarcoma virus (RSV), the early and late promoters from Simian virus 40 (SV40, Fiers et al, Nature, 273, 113, (1978)) or promoters derived from Moloney murine leukemia virus, mouse tumor virus, avian sarcoma viruses, adenovirus II, bovine papilloma virus or polyoma. Preferred bone-related promoters include CMV βActin or type I collagen promoters. In addition to a promoter, the gene may be placed under the control of a ribosome binding site (for bacterial expression), suitable gene control sequence, or regulatory sequences so that the DNA sequence encoding the protein is transcribed into RNA in the host cell transformed by a vector containing this expression construct. In some cases it may be desirable to add sequences which cause the secretion of the polypeptide from the host cell, with subsequent cleavage of the secretory signal. The expression vector may also include a ribosome binding site for translation initiation, a transcription terminator, and an appropriate sequences for amplifying the expression. The expression vector may also include one or more selectable marker genes to provide a specific phenotype for the selection of transformed host cells such as neomycin resistance for eukaryotic cells or ampicillin resistance for E. coli. In addition, the construct may be joined to an amplifiable gene (e.g., DHFR) so that multiple copies of the gene may be made. For appropriate enhancer and other expression control sequences, see also Enhancers and Eukaryotic Gene Expression (Cold Spring Harbor Press, Cold Spring Harbor, N.Y., (1983)).

Vectors and promoters suitable for use in yeast expression are described in EP 73,675A. Examples of vectors suitable for mammalian expression include but are not limited to pCMV SPORT6, pcDNA3.1D/V5-His-TOPO, and pcDNA3.1/CT-GFP-TOPO.

Methods of Inhibiting Ror Expression: The present invention provides methods for inhibiting Ror expression and/or activity that include, but are not limited to, use of antisense nucleic acids, siRNAs, and ribozymes as well as antibodies, peptides and small molecules. Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation.

Methods of Identifying Ror Interacting Proteins: Ror interacting proteins can be identified by methods known to those skilled in the art. For example, immnuprecipitation followed by mass-spectroscopy analysis (Hill et al., J. Biol. Chem. 277, 40735-40741 (2002)) as well as mammalian and yeast two-hybrid systems can be used for studying protein-protein interactions. See, e.g., U.S. Pat. No. 6,251,602, Chien et al., Proc. Natl. Acad. Sci. USA 88, 9578-82 (1991); Fields et al., Trends Genetics 10, 286-92 (1994); Harper et al., Cell 75, 805-16 (1993); Vojtek et al., Cell 74, 205-14 (1993); Luban et al., Cell 73, 1067-78 (1993); Li et al., FASEB J. 7, 957-63 (1993); Zang et al., Nature 364, 308-13 (1993); Golemis et al., Mol. Cell. Biol., 12, 3006-14 (1992); Sato et al., Proc. Natl Acad. Sci. USA, 91, 9238-42 (1994); Coghlan et al., Science, 267: 108-111, (1995); Kalpana et al., Science, 266, 2002-6 (1994); Helps et al., FEBS Lett., 340, 93-8 (1994); Yeung et al., Genes & Devel., 8, 2087-9, (1994); Durfee et al., Genes & Devel., 7, 555-569, (1993); Paetkau et al., Genes & Devel., 8, 2035-45, (1994), Spaargaren et al., Proc. Natl. Acad. Sci. USA, 91, 12609-13, (1994); and Ye et al., Proc. Natl. Acad. Sci. USA, 91, 12629-33 (1994).

Variations of the system are available for screening yeast phagemid (see, e.g., Harper, Cellular Interactions and Development: A Practical Approach, 153-179 (1993); Elledge et al., Proc. Natl. Acad. Sci. USA, 88, 1731-5 (1991)) or plasmid (Bartel, 1993 and Bartel, Cell, 14, 920-4 (1993)); Finley et al., Proc. Natl. Acad. Sci. USA, 91, 12980-4 (1994)) cDNA libraries to clone interacting proteins, as well as for studying known protein pairs.

Yeast strains with integrated copies of various reporter gene cassettes, such as for example GAL.fwdarw.LacZ, GAL.fwdarw.HIS3, or GAL.fwdarw.URA3 (Bartel, in Cellular Interactions and Development: A Practical Approach, 153-179 (1993); Harper et al., Cell, 75, 805-16, (1993); Fields et al., Trends Genetics, 10, 286-92 (1994)) may be co-transformed with two plasmids, each expressing a different fusion protein. One plasmid encodes a fusion between protein “X” and the DNA binding domain of, for example, the GAL4 yeast transcription activator (Brent et al., Cell 43, 729-36 (1985); Ma et al., Cell, 48, 847-53 (1987); Keegan et al., Science, 231, 699-704 (1986)), while the other plasmid encodes a fusion between protein “Y” and the RNA polymerase activation domain of GAL4 (Keegan et al., 1986). The plasmids may be transformed into a strain of the yeast that contains a reporter gene, such as lacZ, whose regulatory region contains GAL4 binding sites. If proteins X and Y interact, they reconstitute a functional GAL4 transcription activator protein by bringing the two GAL4 components into sufficient proximity to activate transcription. Transcription activation is scored by measuring either the expression of β-galactosidase or the growth of the transformants on minimal medium lacking the specific nutrient that permits auxotrophic selection for the transcription product, e.g., URA3 (uracil selection) or HIS3 (histidine selection). See, e.g., Bartel, (1993); Durfee et al., Genes & Devel., 7, 555-569 (1993); Fields et al., Trends Genet., 10, 286-292 (1994); and U.S. Pat. No. 5,283,173.

Additional methodologies for two-hybrid analysis or screening would be apparent to the skilled artisan. See, for example, Finley et al., “Two-Hybrid Analysis of Genetic Regulatory Networks,” in The Yeast Two-Hybrid System (Paul L. Bartel et al., eds., Oxford, (1997)); Meijia Yang, “Use of a Combinatorial Peptide Library in the Two-Hybrid Assay,” in The Yeast Two-Hybrid System (Paul L. Bartel et al., eds., Oxford, (1997)); Gietz et al., “Identification of proteins that interact with a protein of interest: Applications of the yeast two-hybrid system,” Mol. & Cell. Biochem., 172, 67-9 (1997); K. H. Young, “Yeast Two-Hybrid: So Many Interactions, (in) so Little Time,” Biol. Reprod., 58, 302-311, (1998); R. Brent et al., “Understanding Gene and Allele Function with Two-Hybrid Methods,” Annu. Rev. Genet., 31, 663-704 (1997).

Full-length or different portions of Ror may be cloned into yeast two-hybrid vectors. These vectors include but are not limited to pAS, pAS2-1, pGBT9, pGBKT7. The cloned Ror that is expressed as a fusion protein with known binding domain (e.g., GAL4 or LexA) (Serebriiskii I. G., et. al. BioTechniques, 30, 634-655, (2001)) would represent bait for known or unknown protein(s). The cDNA cloned into the activation domain (e.g., GAL4 or VP16) (Serebriiskii I. G., et. al., BioTechniques, 30, 634-655, (2001)) vectors may be from cDNA library made from different osteoblastic cell lines, bone, brain and other tissues where Ror is expressed.

Once the interacting partner is identified the full-length cDNA may be isolated and cloned. In addition, interaction may be confirmed in mammalian two-hybrid system. Also, experiments may be performed that would more clearly define the binding domain within Ror.

If the interacting partner of Ror is a novel gene expressed in osteoblasts, additional experimental approaches may be undertaken to position that protein in bone formation and/or resorption. However, if the Ror interacting partner is a gene with a known biological function, it may indicate the role of Ror interacting partner in modulation of bone formation and/or resorption.

Diagnostic Applications: The Ror molecules of the invention are also useful as markers of bone-related disorders or disease states, as markers for precursors of disease states, as markers for predisposition to disease states, as markers of drug activity, or as markers of the pharmacogenomic profile of a subject. Using the methods described herein, the presence, absence and/or quantity of the Ror molecules of the invention may be detected, and may be correlated with one or more biological states in vivo. For example, the Ror molecules of the invention may serve as biochemical markers for one or more bone disorders or disease states or for conditions leading up to disease states. As used herein, biochemical marker correlates with the absence or presence of a disease or disorder, or with the progression of a disease or disorder (e.g., with the decreased bone mineral density (BMD)). Therefore, these markers may serve to indicate whether a particular course of treatment is effective in lessening a disease state or disorder.

The present invention provides for a method of diagnosing a bone-related disorder in a patient comprising comparing levels of Ror molecule expression and/or activity in a subject with the levels of Ror molecule expression and/or activity in a comparable sample from a normal subject. A sample may be a patient tissue, cell, or body fluid sample.

In addition, the present invention proposes the use of Ror molecules to identify cells and tissues which are responsive to bone-modifying agents in normal state or state of disease by measuring levels of Ror molecule expression and/or activity in these cells or tissues before and after administration of an agent. Changes in Ror molecule expression and/or activity would reflect cell or tissues responsiveness to the agent.

For example, Ror molecules can be used as targets to facilitate design and/or identification of compounds that may be useful as drugs in treating existing osteoporosis or in prevention therapy to reduce risk of fracture and osteoporosis development in pre-menopausal women or other individuals at risk.

These compounds may be used in treatment of osteoporosis in women during menopause to prevent further deterioration of bone and/or to induce new bone formation. It may be given as a mono-therapy and/or in combination with already existing therapies that inhibit bone loss or as an additional therapy to estrogen.

Furthermore, the present invention provides evidence that human Ror2 peaks in proliferating pre-osteoblastic cells. Therefore, Ror2 can be used as a marker for this type of cell in culture or in situ.

Use of Ror Molecules for Evaluating the Efficacy of Drugs: The present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression or activity of Ror molecule in the pre-administration sample; (iii) obtaining one or more post-administration samples from the subject; (iv) detecting the level of expression or activity of Ror molecule in the post-administration samples; (v) comparing the level of expression of or activity of Ror molecule in the pre-administration and post-administration samples; and (vi) altering the administration of the agent to the subject accordingly. For example, increased administration of the agent may be desirable to stronger modulate the expression or activity of Ror, i.e., to increase the effectiveness of the agent. According to such an embodiment, Ror molecule expression or activity may be used as an indicator of the effectiveness of an agent, even in the absence of an observable phenotypic response.

The present invention further provides methods for evaluating the efficacy of drugs and monitoring the progress of patients involved in clinical trials for the treatment of bone-related disorders. Monitoring the influence of agents (e.g., drugs) on the expression or activity of Ror molecules can be applied not only in basic drug screening, but also in clinical trials or during any other period of use. For example, the effectiveness of an agent determined by a screening assay as described herein to increase Ror gene or polypeptide expression levels, can be monitored in clinical trials of subjects exhibiting decreased Ror gene or polypeptide expression levels. Alternatively, the effectiveness of an agent determined by a screening assay to decrease Ror gene or polypeptide expression levels can be monitored in clinical trials of subjects exhibiting increased Ror gene or polypeptide expression levels. In such clinical trials, the expression or activity of Ror gene or polypeptide and other genes and polypeptides that have been implicated in, for example, a Ror-associated disorder can be used as a “read-out” or markers of the phenotype in a particular cell, e.g., a bone cell. In addition, Ror gene or polypeptide expression may be used as a read-out of a particular drug or agent's effect on the state of a bone-related disorder.

For example, and not by way of limitation, genes that are modulated in cells by treatment with an agent (e.g., compound, drug, or small molecule) which modulates Ror molecule expression or activity (e.g., identified in a screening assay as described herein) can be identified. Thus, to study the effect of agents on Ror-associated disorders (e.g., bone-related), for example, in a clinical trial, cells can be isolated and RNA prepared and subjected to gene array analysis (e.g., gene chip analysis). Genes whose expression changed significantly between the untreated and the treated states can serve as markers indicative of the physiological response of the cells to the agent. Accordingly, this response state may be determined before and at various points during treatment of the individual with the agent.

Transgenic Animals: The term “animal” in “transgenic animals” includes all vertebrate animals, except humans. It also includes an individual animal in all stages of development, including embryonic and fetal stages. A “transgenic animal” is an animal containing one or more cells bearing genetic information received, directly or indirectly, by deliberate genetic manipulation at a sub-cellular level, such as by microinjection or infection with recombinant virus. This introduced DNA molecule may be integrated within a chromosome, or it may be extra-chromosomally replicating DNA. The term “transgene” means a DNA sequence introduced into the germ line of an animal by way of human intervention. The term “germ cell-line transgenic animal” refers to a transgenic animal in which the genetic information was introduced into a germ line cell, thereby conferring the ability to transfer the information to offspring. If such offsprings in fact possess some or all of that information, then they, too, are transgenic animals.

The information may be foreign to the species of animal to which the recipient belongs, foreign only to the particular individual recipient, or genetic information already possessed by the recipient. In the last case, the introduced gene may be differently expressed compared to the native endogenous gene.

The genes may be obtained by isolating them from genomic sources, by preparation of cDNAs from isolated RNA templates, by directed synthesis, or by some combination thereof.

To be expressed, a gene is operatively linked to a regulatory region. Regulatory regions, such as promoters, may be used to increase, decrease, regulate or designate to certain tissues or to certain stages of development the expression of a gene. The promoter need not be a naturally occurring promoter.

The methods enabling the introduction of DNA into cells are generally available and well-known in the art. Different methods of introducing transgenes can be used. Generally, the zygote is the best target for microinjection. For example, in the mouse, the male pronucleus reaches the size of approximately 20 μm in diameter, which allows reproducible injection of 1-2 pl of DNA solution. The use of zygotes as a target for gene transfer has a major advantage. In most cases, the injected DNA will be incorporated into the host gene before the first cleavage (Brinster, et al., 1985). Consequently, nearly all cells of the transgenic non-human animal will carry the incorporated transgene. Generally, this will also result in the efficient transmission of the transgene to offspring of the founder since 50% of the germ cells will harbor the transgene. Microinjection of zygotes is a preferred method for incorporating transgenes in practicing the invention.

Retroviral infection can also be used to introduce a transgene into a non-human animal. The developing non-human embryo can be cultured in vitro to the blastocyst stage. During this time, blastomeres may be targets for retroviral infection. Efficient infection of the blastomeres is obtained by enzymatic treatment to remove the zona pellucida. The viral vector system used to introduce the transgene is typically a replication-defective retrovirus carrying the transgene. Transfection is easily and efficiently obtained by culturing the blastomeres on a monolayer of virus-producing cells (Van der Putten, supra; Stewart et al., 1987). Alternatively, infection can be performed at a later stage. Virus or virus-producing cells can be injected into the blastocoele. Most of the founder animals will be mosaic for the transgene since incorporation occurs only in a subset of the cells which formed the transgenic non-human animal. Furthermore, the founder animal may contain retroviral insertions of the transgene at a variety of positions in the genome; these generally segregate in the offspring. In addition, it is also possible to introduce transgenes into the germ line, albeit with low efficiency, by intrauterine retroviral infection of the midgestation embryo (Jahner et al., (1982) supra).

A third type of target cell for transgene introduction is the embryonal stem (ES) cell. ES cells are obtained from pre-implantation embryos cultured in vitro (Evans, M. J., et al., 1981; Bradley, A., et al. 1984; Gossler, et al., 1986; and Robertson, et al., 1986). Transgenes can be efficiently introduced into ES cells by DNA transfection or by retrovirus-mediated transduction. The resulting transformed ES cells can thereafter be combined with blastocysts from a non-human animal. The ES cells colonize the embryo and contribute to the germ line of the resulting chimeric animal (For review see Jaenisch, R., 1988).

General methods for creating transgenic animals are known in the art, and are described in, for example, Strategies in Transgenic Animal Science (Glenn M. Monastersky and James M. Robl eds., ASM Press; Washington, D.C., 1995); Transgenic Animal Technology: A Laboratory Handbook (Carl A. Pinkert ed., Academic Press 1994); Transgenic Animals (Louis Marie Houdebine, ed., Harwood Academic Press, 1997); Overexpression and Knockout of Cytokines in Transgenic Mice (Chaim O. Jacob, ed., Academic Press 1994); Microinjection and Transgenesis: Strategies and Protocols (Springer Lab Manual) (Angel Cid-Arregui and Alejandro Garcia-Carranca, eds., Springer Verlag 1998); and Manipulating the Mouse Embryo: A Laboratory Manual (Brigid Hogan et al., eds., Cold Spring Harbor Laboratory Press 1994). The methods for evaluating the presence of the introduced DNA as well as its expression are also readily available and well-known in the art. Such methods include, but are not limited to, DNA hybridization (southern hybridization) or PCR to detect the exogenous DNA or PAGE and western blots to detect protein.

To determine if Ror plays a role in bone-related disorders, transgenic mice are created that overexpress the full-length gene or any fragment or variants or mutants thereof in all tissues or only in bone. To establish a broad overexpression of Ror in bone as well as non-bone tissues, a ubiquitous promoter (e.g. CMV β-actin) is used. Expression of Ror can be made conditional using the Mifepristone-dependent gene-inducible system. Bone-specific overexpression of the rat cDNA is driven by promoters such as the rat 3.6 kb type I collagen or rat 1.7 kb osteocalcin promoter. The rat 3.6 kb type I collagen promoter is useful for providing early expression in developing bone, whereas the rat 1.7 kb osteocalcin promoter may confer a more bone-restricted pattern of expression. The same constructs are also used to generate transgenic rats. For conditional expression of Ror, the osteocalcin promoter (Capparelli, F. B., Endocrinol., 138, 2109-2116, (1997)) drives the gene-inducible system and the Gal4 minimal promoter drives Ror. Independent transgenic lines are crossed and double-transgenic mice are treated with Mifepristone to regulate Ror expression on demand. Phenotyping of the transgenic animals involves a combination of in vivo and ex vivo assays. An increased BMD provides the proof of concept that Ror molecule is indeed a potential new target for bone-related disorders. Furthermore, these transgenic animals are used to determine if Ror overexpression can rescue ovariectomy-induced osteopenia in rats and mice in these established osteopenia models (Y. P. Kharode et al. J. Bone Min. Res., 14(1), S523, (1999), Y. P. Kharode et al. J. Bone Min. Res., 16 (1), S540, (2001)).

Transgenic animals can be used for the analysis of the bone phenotype of various bones of the skeleton including flat bones (skull, scapula, mandible and ileum) and long bones or axial bones (tibia, femur and humerus) and such with various histomorphometric parameters known in the art.

In addition, Ror transgenic animals can be tested for protection from bone loss in various bone loss models including ovariectomy-induced osteopenia, glucocorticoid-induced osteopenia, and various disuse models and such which are known in the art. The effectiveness of combination of various bone anabolic agents (e.g., identified small molecules) can be investigated and determined to further modify the bone phenotype in an anabolic fashion (synergistically, additively) or in a catabolic manner.

Furthermore, Ror transgenic animals can be used to study the role of Ror in osteoprogenitor and osteoblastic cell activity, proliferation rates and apoptosis rates which collectively influence bone formation.

Ror transgenic animals can be used for identifying agents effective for the treatment of bone related disorders. An agent may be administered to the transgenic animal of present invention. Alteration in bone formation and other bone-related activities in the treated animals can be measured, and compared with bone formation and other bone-related activity in untreated control animals.

Conditional Knockout Animals:

Alternatively, “knock out” animals can be constructed which have a defective or altered gene encoding gene identified in the screen, as a result of homologous recombination between the endogenous gene encoding the gene and altered genomic DNA encoding the same polypeptide introduced into an embryonic cell of the animal. For example, cDNA encoding an identified gene can be used to clone genomic DNA encoding that polypeptide in accordance with established techniques. A portion of the genomic DNA encoding an identified gene can be deleted or replaced with another gene, such as a gene encoding a selectable marker that can be used to monitor integration. Typically, several kilobases of unaltered flanking DNA (both at the 5′ and 3′ ends) are included in the vector (see e.g., Thomas and Capecchi, Cell, 51(3), 503-12, (1987) for a description of homologous recombination vectors). The vector is introduced into an embryonic stem cell line (e.g., by electroporation) and cells in which the introduced DNA has homologously recombined with the endogenous DNA are selected (see e.g., Li, et al., Cell, 69(6), 915-26, (1992). The selected cells are then injected into a blastocyst of an animal (e.g., a mouse or rat) to form aggregation chimeras (see e.g., Bradley, in Teratocarcinomas and Embryonic Stem Cells: A Practical Approach, E. J. Robertson, ed. IRL, Oxford, 113-1521, (1987). A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, by their ability to defend against certain pathological conditions and by their development of pathological conditions due to absence of the identified gene.

Knock-out animals can be used to screen drugs that may influence the biochemical, and pathological parameters relevant to the particular physiological disorder being studied. Cell lines can also be derived from these animals for use as cellular models of the physiological disorder, or in drug screening. For example, a knock-out animal can be developed by introducing a mutation in the Ror1 or Ro2 sequence thereby generating an animal which no longer expresses the functional Ror 1 or Ror2 gene. Such knock-out animals are useful, e.g., for studying the role of the Ror1 or Ro2 in bone related activities or other related physiological functions.

The present invention provides animals in which the Ror gene is conditionally inactivated. Conditional inactivation refers to inactivation of a gene in a selected tissue (i.e. in bone) and/or at a specific time during development. The conditionally inactivated gene is expressed at normal endogenous levels in all other tissues and at all times prior to inactivation. In one embodiment, the present invention provides for the gene information to develop conditional Ror knockout mouse.

A conditional mouse knockout is created that may be dependent on expression of Cre recombinase specifically in bone. Deletion of the targeted allele in bone is performed by crossing gene-targeted animals with transgenic mice expressing Cre specifically in bone. For example, transgenic mice are created where Cre is driven by either the rat 3.6 kb type I collagen promoter or the rat 1.7 kb osteocalcin promoter.

The expected phenotype is one where bone formation is altered. Phenotyping of the targeted animals involves a combination of in vivo and ex vivo assays. The failure to develop normal bone or remodel bone normally provides the role of Ror in bone development and its use as a novel target for bone-related disorders.

Conditional knock-out animals can be used to analyze the bone phenotype of various bones of the skeleton including flat bones (skull, scapula, mandible and ileum) and long bones or axial bones (tibia, femur and humerus) and such with various histomorphometric parameters known in the art.

Furthermore, conditional Ror knockout animals can be used to assess osteoprogenitor and osteoblastic cell activity, proliferation rates and apoptosis rates in the absence of Ror.

The present invention incorporates by reference methods and techniques well known in the field of molecular and cellular biology. These techniques include, but are not limited to techniques described in the following publications: Old, R. W. & S. B. Primrose, Principles of Gene Manipulation: An Introduction To Genetic Engineering (3d Ed. 1985) Blackwell Scientific Publications, Boston. Studies in Microbiology; V.2: 409 pp. (ISBN 0-632-01318-4), Sambrook, J. et al. eds., Molecular Cloning: A Laboratory Manual (2d Ed. 1989) Cold Spring Harbor Laboratory Press, NY. Vols. 1-3. (ISBN 0-87969-309-6), Miller, J. H. & M. P. Calos eds., Gene Transfer Vectors For Mammalian Cells (1987) Cold Spring Harbor Laboratory Press, NY. 169 pp. (ISBN 0-87969-198-0).

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion, embodiments and these examples, one skilled in the art will readily appreciate that many modifications are possible in the exemplary embodiments without materially departing from the novel teachings of this inventions, and without departing from the spirit and scope thereof. Furthermore, one can make various changes to and modifications of the invention to adapt it to various usages and conditions. Accordingly, all such modifications are intended to be included within the scope of this invention as defined in the following claims.

The patents, applications, test methods, and publications mentioned herein are hereby incorporated by reference in their entirety.

### General Methods

Materials and Tissue Culture

Except where noted, tissue culture reagents were purchased from Invitrogen Corporation (Carlsbad, Calif.); other reagents and chemicals were purchased from either Sigma Chemical Co. (St. Louis, Mo.) or Invitrogen. Anti-flag M2 mouse monoclonal antibody, anti-V5 rabbit polyclonal antibody, and anti-flag M2 affinity agarose were obtained from Sigma; anti-HA tag and anti-β-catenin rabbit polyclonal antibodies and anti-phosphotyrosine mouse monoclonal antibody, clone 4G10, were from Upstate Cell Signaling Solutions (Charlottesville, Va.); anti-his mouse monoclonal antibody was from BD Biosciences Clontech (Palo Alto, Calif.); anti-β-actin mouse monoclonal antibody was from Sigma; horseradish peroxidase (HRP)-conjugated secondary antibodies were purchased from Santa Cruz Biotechnology, Inc. (Santa Cruz, Calif.) or Amersham Biosciences (Buckinghamshire, England).

The HOB cell lines were maintained at 34° C. in a 5% CO₂/95% humidified air incubator using DMEM/F-12 medium containing 10% heat-inactivated fetal bovine serum, 1% penicillin-streptomycin, and 2 mM glutaMAX-I. The U2OS human osteosarcoma cells were kept at 37° C. in McCoy's 5A Modified Medium, containing 10% heat-inactivated fetal bovine serum, 1% penicillin-streptomycin, and 2 mM glutaMAX-I. COS-7 cells were maintained at 37° C. in DMEM containing 10% heat-inactivated fetal bovine serum, 1% penicillin-streptomycin, and 2 mM GlutaMAX-I.

Plasmids

HA epitope-tagged human Wnt-1 in pUSEamp (Wnt-1-HA), HA epitope-tagged human Wnt-3 in pUSEamp (Wnt-3-HA) and pUSEamp(+) were obtained from Upstate Biotechnology; pcDNA3.1(+) was from Invitrogen; CMV promoter-driven β-galactosidase reporter gene (pCMVβ) was from BD Biosciences Clontech. Human SFRP-1 has been described in WO 01/19855.

Luciferase reporter gene containing 16 copies of the T-cell factor (TCF) DNA binding site fused 5′ to a minimal Thymidine Kinase (TK) promoter (16×TCF-luc) was constructed as follows. Oligonucleotides were generated containing the TCF DNA binding sites originally identified in the TCR-alpha enhancer, Waterman, M. L., et al., Genes Dev., 5, 656-669, (1991), the CD3-e enhancer, van de Wetering M., et al., EMBO J., 10, 123-132, (1991), and the consensus TCF DNA binding site Korinek, V., et al., Science, 275, 1784-1787, (1997).

These oligonucleotides were (the TCF binding sites are underlined):

| | |
|---|---|
| 5′-CTAGCGAGAACAAAGGAGATTCAAAGGAGATCAAAGGAGATCAAAGGACTAGTTC-3′, | (SEQ ID NO:1) |
| and, | |
| 5′-TCGAGAACTAGTCCTTTGATCTCCTTTGATCTCCTTTGAATCTCCTTTGTTCTCG-3′ | (SEQ ID NO:2) |

The oligonucleotides were phosphorylated using T4 polynucleotide kinase, heated for 10 min at 80° C., and allowed to anneal. The annealed double stranded oligonucleotides contained NheI and XhoI compatible sequences at the 5′ and 3′ ends, respectively. The annealed oligonucleotides were cloned upstream of a TK-luciferase reporter into NheI- and XhoI-digested pGL3 (Promega Corporation, Madison, Wis.) to generate 4×TCF-luc. The TCF binding sites and TK promoter were confirmed by DNA sequencing. A plasmid with 8 copies of the TCF DNA binding site (8×TCF-luc) was then developed by ligating NheI-BamHI and SpeI-BamHI fragments from the 4×TCF-luc. Similarly, a plasmid with 16 copies of the TCF DNA binding site was prepared utilizing the 8×TCF-luc. All plasmids were verified by sequencing.

Flag-tagged human Ror1 (Ror1-flag) was constructed as follows. The human Ror1 was cloned from human uterus RNA and inserted into KpnI and NotI sites of pcDNA3.1(+) to obtain hRor1-pcDNA3. This clone was further modified by PCR-mediated mutagenesis. The 3′ untranslated region was removed and the flag epitope tag (codons underlined) was added to the 3′-end of cDNA before the stop codon and the NotI site using the bottom strand primer: 5′-TGAGCGCGGCCGCTGCTCACTTGTCATCGTCGTCCTTGTAGTCCAGTTCTGCA GAAATCATAGAT-3′ (SEQ ID NO: 14). The top strand primer was complementary to the internal region of Ror1, which contained a BamHI site: 5′-CTCATCAGGATCCMTCAGG-3′ (SEQ ID NO: 13). The amplified DNA was cut with BamHI and NotI and cloned into BamHI- and NotI-digested hRor1-pcDNA3. The intended changes were verified by sequencing.

Flag-tagged human Ror2 (Ror2-flag-pcDNA3) was made as follows. The partial clone for human Ror2 containing the codons 58-2296 was obtained from Invitrogen's IMAGE clone collection (clone ID 3146587) in pCMV-Sport6 and verified by sequencing both strands. The 3′ portion (codons 2297-2832) was produced by RT-PCR using HOB-03-C5 RNA as a template and Ror2-specific primers. The top strand primer was complementary to codons 2192-2216 of Ror2 and contained an internal XmnI site: 5′-AGTTCCCCAGCCGGCGGCCCCGCTT-3′ (SEQ ID NO: 15) and the bottom strand primer contained an XbaI site adjacent to the stop codon (boldface type): 5′-TACGATTCTAGATGTCMGCTTCCAGCTGGACTTGGG-3′ (SEQ ID NO: 16). The amplified DNA was digested with XmnI and XbaI to obtain the 3′-end of Ror2. The 5′ porion was excised from the IMAGE clone with NotI and XmnI, and both fragments were cloned into NotI- and XbaI-digested pcDNA3.1(+) to obtain Ror2-3′-pcDNA3. The first 57 codons of Ror2 were also produced by RT-PCR using HOB-03-C5 RNA as a template and Ror2-specific primers. The top strand primer contained a KpnI site 5′ to an ATG codon (boldface type): 5′-GACCTTGGTACCATGGCCCGGGGCTCGGCGCT-3′ (SEQ ID NO: 17); and the bottom strand primer was complementary to the internal region of Ror2, which contained a BamHI site: 5′-TCGTTCGGATCCAGMCCTCCAC-3′ (SEQ ID NO: 18). The amplified DNA was digested with KpnI and BamHI and cloned into the KpnI- and BamHI-digested Ror2-3′-pcDNA3 to obtain Ror2-pcDNA3. The entire coding region was verified by sequencing both strands. Subsequently, the flag-tagged human Ror2 was generated by PCR-mediated mutagenesis. The flag epitope tag (codons underlined) was added to the 3′-end of hRor2 cDNA before the stop codon (boldface type) and the XbaI site using the bottom strand primer: 5′-CTGGAATCTAGATCACTTGTCATCGTCGTCCTTGTAGTCAGCTTCCAGCTGGAC TTGGGCC-3′ (SEQ ID NO: 20). The top strand primer was complementary to the internal region of Ror2, containing an AleI site: 5′-GCTCACACCACAGTGGCAGTGG-3′ (SEQ ID NO: 19). The amplified DNA was cut with AleI and XbaI and cloned into AleI- and XbaI-digested Ror2-pcDNA3. The intended changes were verified by sequencing.

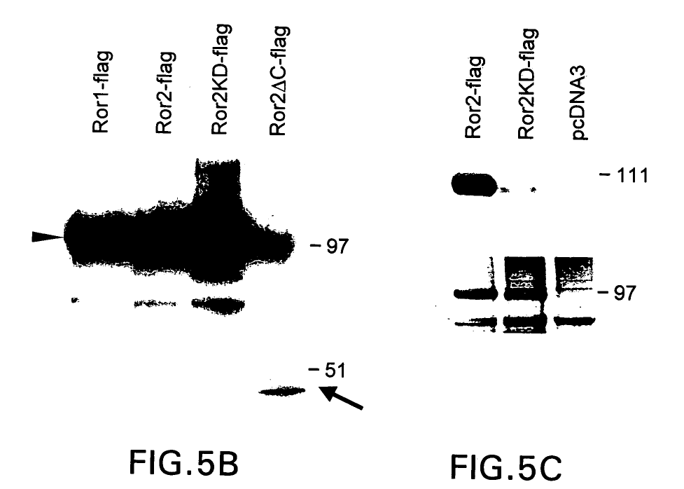

Flag-tagged Ror2 ‘kinase-dead’ mutant (Ror2KD-flag) diagrammed in FIG. 5A was designed based on published evidence (Hikasa H, Shibata M, Hirati I, Taira M, Development, 129, 5227-5239, (2002)). The three point mutations of lysines 507, 510 and 512 to isoleucines were introduced into Ror2-Flag-pcDNA3 by PCR-mediated mutagenesis using the QuikChange XL Site-Directed Mutagenesis Kit (Stratagene, La Jolla, Calif.) according to the manufacturer's instructions. The top strand primer containing the intended mutations (bold) was: 5′-GGCTGTGGCCATCATMCGCTGATAGACATAGCGGAGGGGC-3′ (SEQ ID NO: 21) and the bottom strand primer was complementary to the top: 5′-GCCCCTCCGCTATGTCTATCAGCGTTATGATGGCCACAGCC-3′ (SEQ ID NO: 22). After the intended changes were verified by sequencing, the mutated portion was cut out with BsrGI and EcoRI and cloned into BsrGI- and EcoRI-digested Ror2-flag-pcDNA3.

Flag-tagged Ror2 truncation mutant (Ror2ΔC-flag) diagrammed in FIG. 5A was constructed by PCR-mediated mutagenesis. The flag epitope tag (codons underlined) was introduced after codon 1284 just beyond COOH terminus of the transmembrane domain followed by the stop codon (boldface type) and the XbaI site using the bottom strand primer: 5′-GACCTTTCTAGATTACTTGTCATCGTCGTCCTTGTAGTCGCACATGCAAACCAA GAAGAAAAGGC-3′ (SEQ ID NO: 24). The top strand primer was complementary to the internal region of Ror2, 5′ to SphI site: 5′-CCTTCTGCCACTTCGTGTTTCCTCT-3′ (SEQ ID NO: 23). The amplified DNA was digested with SphI and XbaI, cloned into SphI- and XbaI-digested Ror2-pcDNA3, and the intended changes were verified by sequencing.

V5- and his-tagged intracellular domain of human Notch2 (bp 5125-7431) was constructed by PCR-mediated mutagenesis. The 5′ end (bases 5125-5906) was produced by RT-PCR from human fetal brain and lung RNA using the top strand primer containing EcoRI site followed by ATG codon: 5′-CATATGMTTCATGACCAAGCATGGCTCTCTCTGGCTGCCT-3′ (SEQ ID NO: 45); and the bottom strand primer complementary to the internal region of Notch2 containing the BclI site: 5′-CGCTTGGCAGTTGATCAGTTCTG-3′ (SEQ ID NO: 46). The 3′ portion containing the codons 5907-7425 was obtained from Invitrogen's IMAGE clone collection (clone ID 5529009) and amplified by PCR. The top strand primer containing BclI site was 5′-GAATGGTGGCAGAACTGATCAACTG-3′ (SEQ ID NO: 47); and the bottom strand primer containing NotI site was 5′-GATATGCGGCCGCCGCATAAACCTGCATGTTGTTGTGTG-3′ (SEQ ID NO: 48). The 5′ and 3′ portions were cloned in frame into EcoRI and NotI sites of pcDNA3.1N5-His (Invitrogen) incorporating V5 and His tags into the protein sequence.

RNA Isolation

The HOB cells were seeded at 9.8×10⁴ cells/cm², allowed to attach overnight at 34° C., and transferred to 39° C. in DMEM/F-12 medium containing 0.25% (wt/v) bovine serum albumin (Serologicals Proteins, Inc, Kankakee, Ill.), 1% penicillin-streptomycin, 2 mM glutaMAX-I, 50 μg/ml ascorbate-2-phosphate (Wako Pure Chemical Industries, Ltd., Osaka, Japan), and 10 nM menadione sodium bisulfite (vitamin K₃). Forty-eight hours later, the cells were washed in phosphate-buffered saline (PBS) and the whole-cell RNA was isolated with TRIzol reagent (Invitrogen) per manufacturer's protocol. Subsequently, the polyA⁽⁺⁾ RNA fraction was extracted using Oligotex mRNA Maxi Kit (Qiagen, Valencia, Calif.) following the manufacturer's instructions.

Transient Transfections and Western Immunoblotting

COS-7 or U2OS cells were seeded at ˜80% confluent density and transfected 24 h later with 40 μg of total plasmid DNA per 143 cm² using FuGENE 6 transfection reagent (Roche Molecular Biochemicals, Indianapolis, Ind.) per manufacturer's instructions. After 24-72 h, cells were solubilized in lysis buffer (150 mM NaCl, 50 mM Tris-HCl, pH 7.5, 1 mM EDTA, 1% Triton X100, 20 mM NaF, 2 mM sodium vanadate, protease inhibitor cocktail (Sigma), 250 μM phenylmethylsulfonyl fluoride). For analysis of HOB-01-09 cell lines, cells were grown to confluence prior to solubilization in lysis buffer. The extracts were clarified by centrifugation at 500,000×g for 30 min at 4° C. For assessing β-catenin stabilization, U2OS cells were plated at −80% confluent density in 6-well plates and transfected 24 h later using 6.9 μg of total plasmid DNA and Lipofectamine 2000 transfection reagent per manufacturer's protocol. After 24 h, the cytoplasmic protein extracts were prepared by solubilization in hypotonic buffer (10 mM Tris-HCl, pH 7.4, 200 μM MgCl₂; protease and phosphatase inhibitor cocktails (both from Sigma)) followed by 40 passes in Dounce homogenizer and centrifugation at 100,000×g for 90 min at 4C. For immunoblotting, 50 μg of total cell lysates or 19 μg of cytoplasmic extract was resolved by SDS-PAGE under denaturing and reducing conditions before transfer onto 0.45 μm nitrocellulose membranes. Membranes were blocked in PBS containing 0.1% Tween-20 and 5% blotto (Santa Cruz) followed by incubation for 2 h at 25° C. with primary antibody (anti-flag epitope tag, 10 μg/ml; anti-his tag, 1.4 μg/ml; anti-HA tag, 2 μg/ml; anti-phosphotyrosine, 2 μg/ml; anti-β-catenin 1.5 μg/ml; anti-β-actin 1:5000; anti-V5, 3 μg/ml; or anti-Ror2, 1 μg/ml). After addition of HRP-conjugated secondary antibodies at 1:2000 for 1 h at 25C, the membranes were analyzed by enhanced chemiluminescence (Amersham Biosciences, Buckinghamshire, England) followed by exposure to x-ray film. Where indicated, the membranes were stripped in stripping buffer (62.5 mM Tris-HCl (pH 7.5), 2% SDS, 0.1% β-mercaptoethanol) for 30 min at 55° C. and reprobed with the next set of antibodies.

Immunoprecpitation and In Vitro Autophosphorylation Assay

U2OS cells were transfected as for western immunoblotting and 24 h later solubilized in lysis buffer and clarified by centrifugation at 500,000×g for 30 min at 4° C. One milligram of total cell lysates was incubated with 50 μl of M2 flag affinity agarose (Sigma) for 1 h with rotation at 4° C. The beads were collected by centrifugation, washed three times in lysis buffer containing 350 mM NaCl and three times in lysis buffer, boiled in 50 μl of 2×LDS-PAGE buffer with reducing agent (Invitrogen), and the solubilized proteins were separated by SDS-PAGE. The gels were transferred onto 0.45 μm nitrocellulose membrane before detection with each specific antibody.

For autophosphorylation assays, 3 mg of total cell lysates in kinase lysis buffer (lysis buffer without EDTA) were incubated with 50 μl of M2 flag affinity agarose (Sigma) for 1 h with rotation at 4° C. The extracts were clarified by centrifugation at 500,000×g for 30 min at 4° C. Three milligrams of total cell lysates were incubated with 50 μl of M2 flag affinity agarose (Sigma) for 1 h with rotation at 4° C. The beads were collected by centrifugation, washed three times in kinase lysis buffer containing 350 mM NaCl, three times in kinase lysis buffer and two times in kinase reaction buffer (10 mM MgCl₂, 50 mM Tris-HCl pH 7.5, 1 mM Dithiotriethol). Ten percent of the beads were set aside for SDS-PAGE analysis and the rest were resuspended in 50 μl of kinase reaction buffer containing 1 mM ATP and 15 μCi [γ-³²P] ATP. The kinase reaction was allowed to proceed for 30 min at 30° C. and stopped by boiling in 1×LDS buffer plus reducing agent (Invitrogen). Proteins were resolved by SDS-PAGE, transferred onto 0.45 μm nitrocellulose membranes and exposed to X-ray film for 12 h at −80° C. with intensifying screens. Where indicated, the membrane was subsequently probed with anti-flag antibody as described in Western Immunoblotting.

Statistical Analysis

Data are presented as means±standard error (SE). Statistical significance was determined using Student's t test. Results were considered statistically different when p<0.05.

### Example 1

### Expression of the Ror2 Gene Decreases During Late Stages of Human Osteoblast Differentiation and is Suppressed by SFRP-1

The discovery that Ror2 is involved in human osteoblast differentiation was made using the gene chip technology. For these experiments, the polyA⁽⁺⁾ RNA samples from proprietary HOB cell lines (HOB-03-C5, HOB-03-CE6, HOB-02-C1, HOB-01-C1, and HOB-05-T1), representing distinct stages of osteoblast differentiation (proliferative, early and mature osteoblastic, pre-osteocytic, and osteocytic, respectively) were subjected to gene chip analysis using the GIHuman1a chip enriched in bone and cartilage cDNAs. Target complementary RNA (cRNA) preparation and hybridization to Affymetrix GeneChips were done essentially as described in, Hill A A, et al., Genome Biol., 2, (2001), Hill A A, et al., Science, 290, 809-12, (2000). Eleven biotin-labeled control cRNA transcripts were spiked into hybridization solution at known concentrations and used to generate a calibration curve between Average Difference (AD) values and picomolarity. Picomolarity values were converted to Frequency Per Million based on the assumption that the average cRNA length is 1 kB. Chips were scanned and analyzed using the Affymetrix MAS 4.0 software and AD values for each probe set were calculated per Affymetrix instructions (Santa Clara, Calif.). AD values were then used to derive Frequency Per Million values for each probe set using the calibration curve obtained from control transcripts. This analysis identified 29 kinases that changed over 2-fold during osteoblast differentiation.

Bodine et al. have previously shown that an antagonist of Wnt signaling, SFRP-1, promotes osteoblast apoptosis (Bodine, P. V. N., et al., WO 01/19855, Bodine, P. V. N., et al., The 23^{rd} Meeting of the ASBMR. Phoenix, Ariz., 2001), and that mice engineered to lack SFRP-1 (generated in collaboration with Lexicon Genetics, Inc., Woodlands, Tex.) have increased bone formation, Bodine, P. V. N., et al., The 24^{th} Meeting of the ASBMR. San Antonio, Tex., 2002. To characterize SFRP-1 mechanism of action, Gene chip analysis was performed on polyA⁽⁺⁾ RNA from HOB-01-09 pre-osteocytic cells stably over-expressing either SFRP-1 or a control vector. Gene chip technology was also used to look at the transcription profiles of SFRP-1 knockout mice compared to wild type controls.

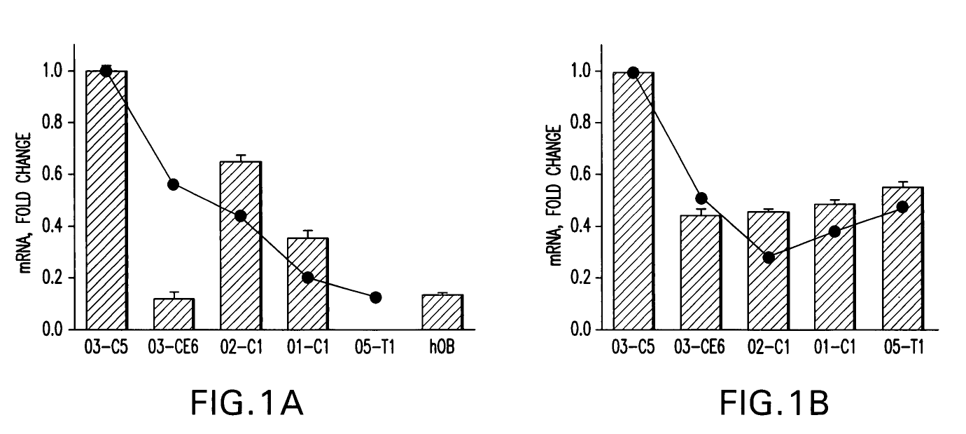

Comparing the results of the gene chip screens, Applicants have discovered that the expression of one kinase was not only changed during osteoblast differentiation, but was also regulated by SFRP-1 expression. This was Ror2 kinase whose mRNA expression decreased as osteoblasts progressed towards osteocytic phenotype (FIG. 1A), in parallel to an increase in SFRP-1 expression. The change in Ror2 mRNA levels was confirmed by RT-PCR analysis (FIG. 1A). The same PolyA⁽⁺⁾ RNA samples used for gene chip analysis, were subjected to the real-time RT-PCR using the ABI PRISM 7700 Sequence Detection System (Applied Biosystems, Foster City, Calif.) per manufacturer's instructions. Sequences of primers and probes used are listed in Table 1.

TABLE 1

Primers and Probes Used in the Real-Time PCR Analysis of
Human Ror1

| | | |
|---|---|---|
| Forward primer, 2993-3013 | 5′-GTCGACTAGCACTGGCCATGT-3′ | SEQ ID NO:25 |
| Reverse primer, 3049-3074 | 5′-CATGTGTGGTAGTAAAGGAATATTTGC-3′ | SEQ ID NO:26 |
| Probe, 3018-3044 | 5′-AGCTTGCCCTCATCAGGATCCAATCAG-3′ | SEQ ID NO:27 |

Primers and Probes Used in the Real-Time PCR Analysis of
Human Ror2

| | | |
|---|---|---|
| Forward primer, 1149-1169 | 5′-CGTACGCATGGAACTGTGTGA-3′ | SEQ ID NO:28 |
| Reverse primer, 1239-1259 | 5′-CAAGCGATGACCAGTGGAATT-3′ | SEQ ID NO:29 |
| Probe, 1174-1198 | 5′-CCCTCGTGTAGTCCCCGAGACAGCA-3′ | SEQ ID NO:30 |

Probes were obtained from Applied Biosystems and labeled with the reporter fluorescent dye FAM. Primers and probe labeled with the reporter fluorescent dye VIC, specific for 18S rRNA, were purchased from Applied Biosystems and included in the reactions as an internal control. The reverse transcriptase step was performed at 48° C. for 30 min and the cDNA was amplified for 40 cycles at the following conditions: 95° C. for 15 s and 60° C. for 1 min. The mRNA amount for each gene was calculated using the Standard Curve Method described in User Bulletin #2 (Applied Biosystems) and normalized to the expression of 18S rRNA.

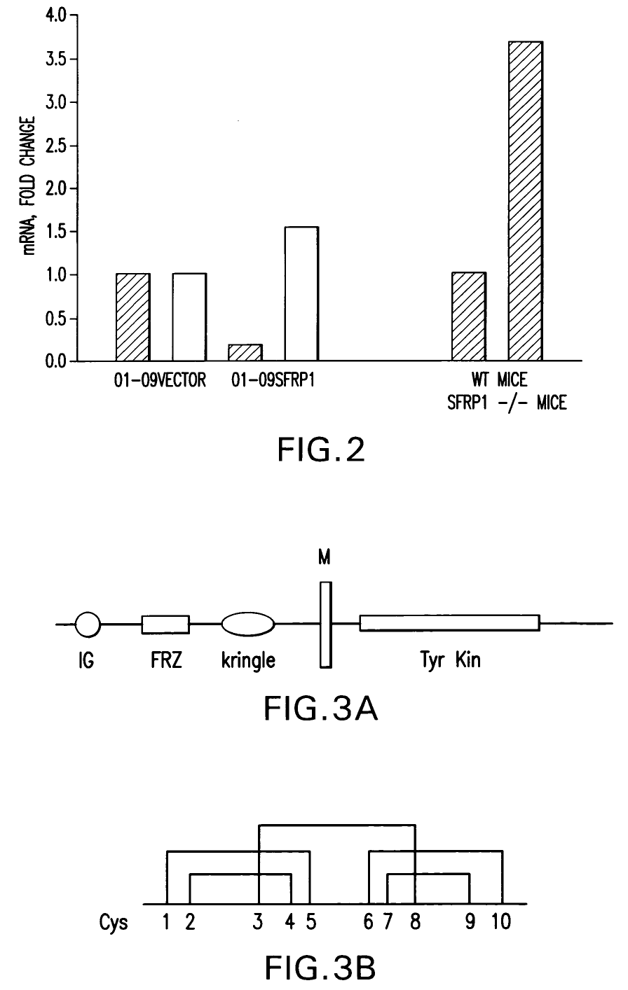

RT-PCR was also used to verify expression of Ror2 in primary human osteoblasts (FIG. 1A). Levels of Ror2 expression in human osteoblasts were similar to those observed in early osteoblastic cells in the HOB model. Overexpression of SFRP-1 in HOB-01-09 cells resulted in strong suppression of Ror2 mRNA levels, whereas calvariae of SFRP-1-disrupted mice expressed two-fold more Ror2 message than did the wild type controls (FIG. 2). Expression of the only other known member of the Ror family, Ror1, also decreased during osteoblast differentiation (FIG. 1B), but did not appear to be regulated by SFRP-1. Indeed, Ror1 levels were below the detection limit in calvariae of both wild type as well as SFRP-1-null mice and were unchanged upon SFRP-1 overexpression in HOB-01-09 cells (FIG. 2).

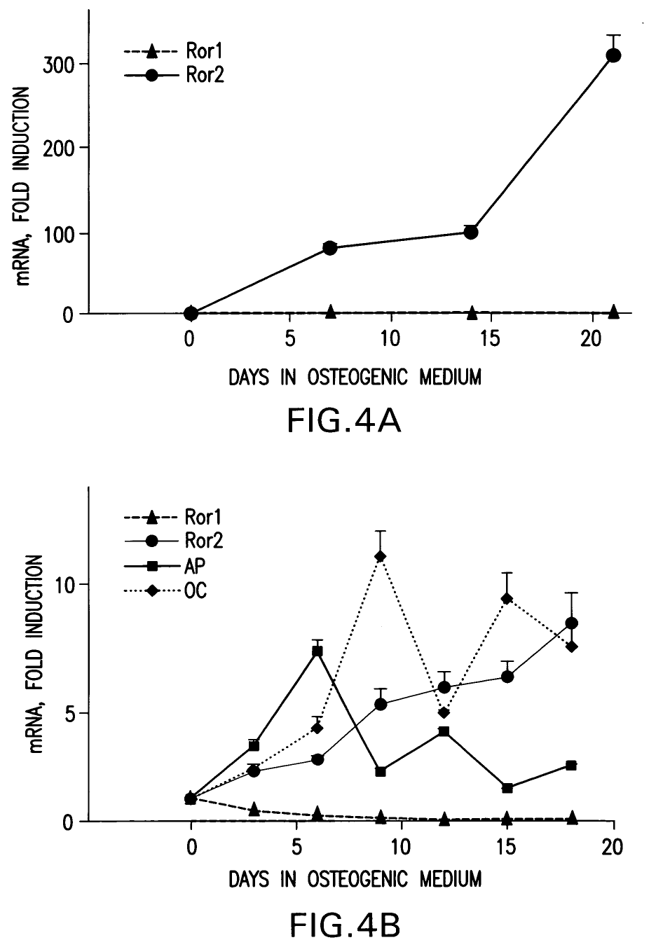

DeChiara et al., Nature Genetics, 24, 271-4, (2000) and Takeuchi et al., Genes to Cells, 5, 71-8, (2000) reported that disruption of the Ror2 gene in mice leads to widespread skeletal abnormalities. In addition, Masiakowski et al., Journal of Biological Chemistry, 267, 26181-90, (1992) reported that Ror1 and 2 kinases possess a Frizzled-related domain homologous to that of SFRP-1 that mediates binding to Wnt proteins (FIG. 3). Roszmusz et al., Journal of Biological Chemistry, 276, 18485-90, (2001) reported that the localization of disulfide bonds within the Frizzled domain of Ror1 is identical to that of SFRP-1. These data suggested that Ror2 participates in osteoblast differentiation and that it is involved in Wnt and SFRP-1 signaling pathways.

### Example 2

### Expression of Ror2 Gene Increases During Initial Stages of Human Osteoblast Differentiation and During Initial and Late Stages of Mouse Osteoblast Differentiation

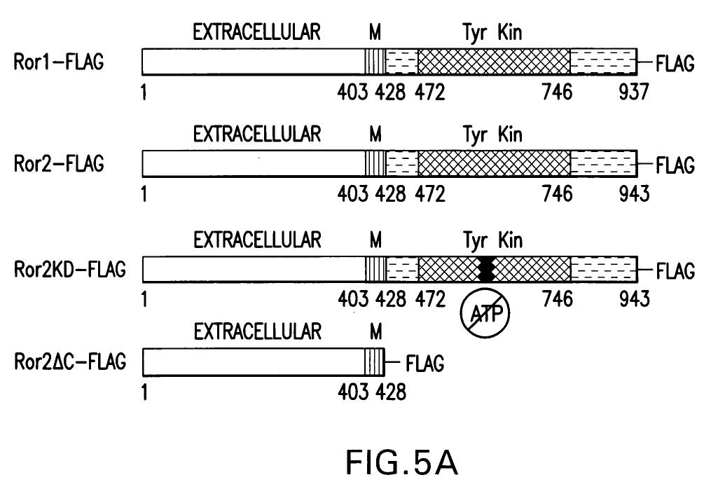

To assess expression of Ror kinases during early stages of human osteoblast differentiation, osteoblastic differentiation of pluripotent human mesenchymal stem cells (hMSC, BioWhittaker, Inc., San Diego, Calif.) was analyzed. The hMSC were maintained at 37° C. in a 5% CO₂/95% humidified air incubator using phenol red-free DMEM medium containing 10% heat-inactivated fetal bovine serum (BioWhittaker), 1% penicillin-streptomycin, and 2 mM glutaMAX-I (growth medium). The hMSC were seeded at 992 cells per well in 96-well plates and 24 h later (day 0) osteogenesis was induced by addition of osteogenic medium (0.1 μM dexamethasone, 0.05 mM ascorbic acid and 10 mM β-glycerophosphate in growth medium) for 21 days. Every 7 days, the total cellular RNA was isolated and expression of Ror genes was assessed by real-time RT-PCR analysis as described in Example 1 using primers and probes listed in Table 1. As shown in FIG. 4A, Ror2 expression increased dramatically during the time course of differentiation with over 300-fold induction observed by day 21. In contrast, Ror1 expression did not change during the entire time course of differentiation (FIG. 4A).

In addition, Ror expression during ascorbic acid-induced differentiation of mouse MC3T3-E1 osteoblast-like cells was monitored. The MC3T3-E1 cells were maintained at 37° C. in a 5% CO₂/95% humidified air incubator in MEM medium containing 10% heat-inactivated fetal bovine serum, 1% penicillin-streptomycin, and 2 mM glutaMAX-I. Cells were seeded at 3×10⁶ cells per dish in 100 mm plastic culture dishes and 72 h later (at confluence, day 0) differentiation was induced by addition of growth medium supplemented with 10 mM β-glycerophosphate and ascorbic acid (12.5 μg/ml for the first feeding and 25 μg/ml for subsequenct feedings). The medium was changed every 48 h. Whole-cell RNA was isolated every 3 days and real-time RT-PCR was performed as described in Example 1 except using primers and VIC-labeled probe specific for rodent GAPDH (Applied Biosystems) as an internal control. Sequences of primers and probes used for Ror analysis are listed in Table 2.

TABLE 2

Primers and Probes Used in the Real-Time PCR Analysis of
Mouse Ror1

| | | |
|---|---|---|
| Forward primer, 2350-2370 | 5′-CCCCGATTTCCCAATTACATG-3′ | SEQ ID NO:31 |
| Reverse primer, 2402-2421 | 5′-GCCAATGAAACCAGCGATCT-3′ | SEQ ID NO:32 |
| Probe, 2373-2395 | 5′-CCCGAGCCAAGGGATTACACCCC-3′ | SEQ ID NO:33 |

Primers and Probes Used in the Real-Time PCR Analysis of
Mouse Ror2

| | | |
|---|---|---|
| Forward primer, 364-386 | 5′-ATCCAAGACCTGGACACAACAGA-3′ | SEQ ID NO:34 |
| Reverse primer, 429-448 | 5′-GAACCCCAGTGGCAGTGATG-3′ | SEQ ID NO:35 |
| Probe, 400-424 | 5′-TCAGCCCGTTGGTAGCCACACACTG-3′ | SEQ ID NO:36 |

The progression of MC3T3 differentiation to osteoblastic phenotype was monitored by analyzing expression of two specific osteoblastic markers: alkaline phosphatase (AP) and osteocalcin (OC) by RT-PCR with primers and probes listed in Table 3.

TABLE 3

Primers and Probes Used in the Real-Time PCR Analysis of
Mouse alkaline phosphatase

| | | |
|---|---|---|
| Forward primer, 1354-1373 | 5′-GAGACCCACGGTGGAGAAGA-3′ | SEQ ID NO:37 |
| Reverse primer, 1445-1464 | 5′-GGAGGCATACGCCATCACAT-3′ | SEQ ID NO:38 |
| Probe, 1416-1442 | 5′-CGGCGTCCATGAGCAGAACTACATTCC-3′ | SEQ ID NO:39 |

Primers and Probes Used in the Real-Time PCR Analysis of
Mouse osteocalcin

| | | |
|---|---|---|
| Forward primer, 78-96 | 5′-CGGCCCTGAGTCTGACAAA-3′ | SEQ ID NO:40 |
| Reverse primer, 124-145 | 5′-GCCGGAGTCTGTTCACTACCTT-3′ | SEQ ID NO:41 |
| Probe, 98-121 | 5′-CCTTCATGTCCAAGCAGGAGGGCA-3′ | SEQ ID NO:42 |

The time course of AP and OC expression shown in FIG. 4B reveals that MC3T3 cells reach mature osteoblastic phenotype characterized by the highest levels of AP expression after 6 days in osteogenic medium and enter matrix mineralization stage with elevated OC after 9 days. Ror2 expression increased throughout the entire time course of differentiation (FIG. 4B). In contrast, Ror1 expression gradually decreased over the same time course and dropped to about 20% of its original value by day 19 (FIG. 4B).

Thus, in human osteoblasts, Ror2 expression increases during early stages of differentiation, peaks in proliferating pre-osteoblasts and then declines during terminal osteocytic differentiation. In murine osteoblasts, Ror2 expression increases throughout both the early and late stages of differentiation.

### Example 3

### Human Ror1 and Ror2 Cloning and Expression

Human Ror1 and Ror2 were cloned in collaboration with Invitrogen Corporation (Carlsbad, Calif.), and are presented as SEQ ID NO:3 and 5 respectively. The Ror1 sequence had the following substitutions: T590C (silent), T1580G (silent), T1963C (M518T), and A3142G (K911R). The Ror2 sequence differs from that disclosed in U.S. Pat. No. 5,843,749 by two nucleotides: C2088T (silent) and G2455A (V8191). The numbering in the U.S. Pat. No. 5,843,749 differs due to variability in the length of the 5′ UTRs and can be obtained by subtracting 35 for Ror1 and by adding 199 for Ror2.

The expression plasmids for full-length Ror1 and Ror2 and for two Ror2 mutants were generated (FIG. 5A). In the first Ror2 mutant (Ror2KD), 3 lysines at positions 504 (in the putative ATP binding domain), 507 and 509 were replaced with isoleucines and in the second mutant (Ror2ΔC) the entire cytoplasmic portion including the tyrosine kinase homology domain was deleted. All four expression plasmids contained a COOH-terminal flag epitope tag for protein identification. Western immunoblotting showed that Ror1-flag, Ror2-flag, and Ror2KD-flag express at high levels in U2OS osteosarcoma cells, whereas Ror2ΔC-flag exhibits lower level of expression (FIG. 5B). Both Ror2-flag and Ror2KD-flag could be precipitated out of U2OS extracts on flag affinity agarose (FIG. 5C, bottom panel). However, Ror2KD mutant failed to phosphorylate itself in the in vitro autophosphorylation assay (FIG. 5C, top panel) confirming that it has lost its kinase activity. Flag immunoprecipitation and in vitro autophosphorylation assays were performed as described in General Methods.

### Example 4

### Ror2 Kinase Inhibits Wnt-3, but Potentiates Wnt-1 Activity

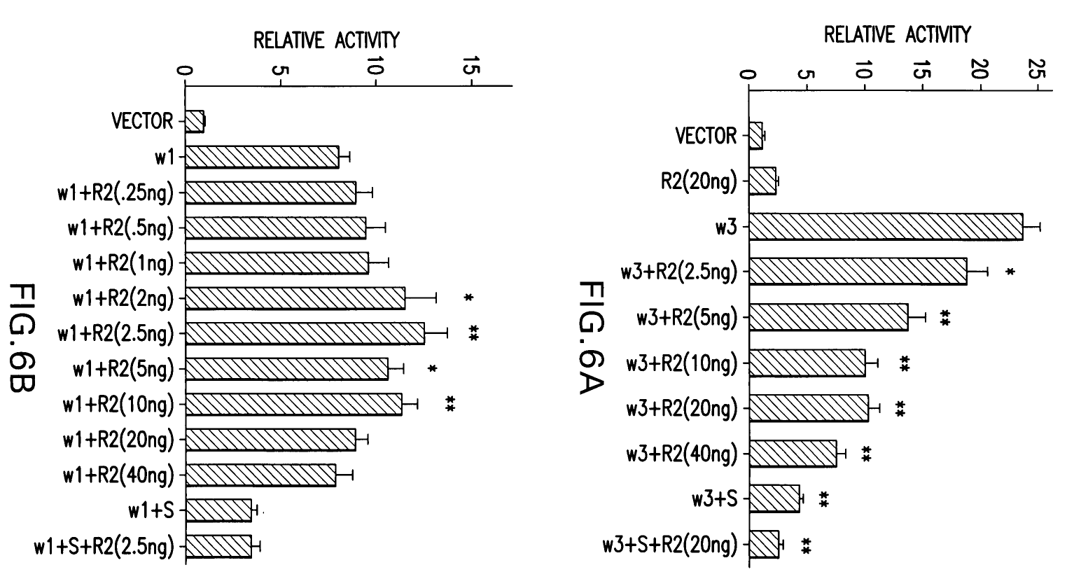

To assess if Ror2 kinase participates in Wnt signaling, a luciferase reporter assay was performed. The U2OS cells were seeded at 20,000 cells per well in 96-well plates and transfected 24 h later in medium without antibiotics using 0.16 μl per well of Lipofectamine 2000 transfection reagent (Invitrogen) per manufacturer's protocol. Combinations of the following DNAs were used per well, adjusted to 230 ng with pcDNA3.1(+): 180 ng 16×TCF-luc (described in Plasmid section of General Methods); 5 ng Wnt-1-HA or Wnt-3-HA; 15 ng SFRP-1; 5 ng pCMVβ; the indicated amounts of Ror1-flag, Ror2-flag or Ror2ΔC-flag. The medium was changed 4 h after the addition of DNA and 24 h later, following aspiration of medium and rinsing in PBS, the cells were lysed and extracts were assayed for luciferase activity using the Luciferase Assay Reagent (Promega) and for β-galactosidase (β-gal) activity using Galacto-Light (Applied Biosystems). Light emission was measured by MicroLumat LB 96P luminometer (EG&G Berthold, Bandoora, Australia) by integration over 10 s for luciferase and 5 s for β-gal. The light emission values obtained for luciferase were normalized to those for β-gal. Expression of a luciferase reporter gene containing 16 copies of the Wnt-responsive TCF binding site fused 5′ to a minimal Thymidine Kinase promoter was stimulated over 20-fold after co-transfection of U2OS cells with Wnt-3-HA construct (FIG. 6A). Co-transfection with Ror2-flag dose-dependently inhibited Wnt-3-induced activation of the promoter with the IC50=5.8 ng/well of a 96-well plate and the maximum inhibition of 68% (FIG. 6A). IC50 refers to the amount of agent required to achieve 50% of the maximal inhibition of the activity observed in presence of Wnt alone. Ror2-flag by itself had no effect on the promoter activity (FIG. 6A). SFRP-1, a potent inhibitor of Wnt signaling, suppressed Wnt-3-HA activity, and together SFRP-1 and Ror2-flag suppressed it even further. By contrast, when the same promoter was activated by Wnt-1-HA, Ror2-flag potentiated Wnt-1 activity with a biphasic dose response (FIG. 6B). Addition of SFRP-1 overcame this potentiation and inhibited luciferase activity to the same level as in presence of Wnt-1-HA alone (FIG. 6B).

### Example 5

### Ror1 Kinase Inhibits Wnt-3, but has No Effect on Wnt-1 Activity

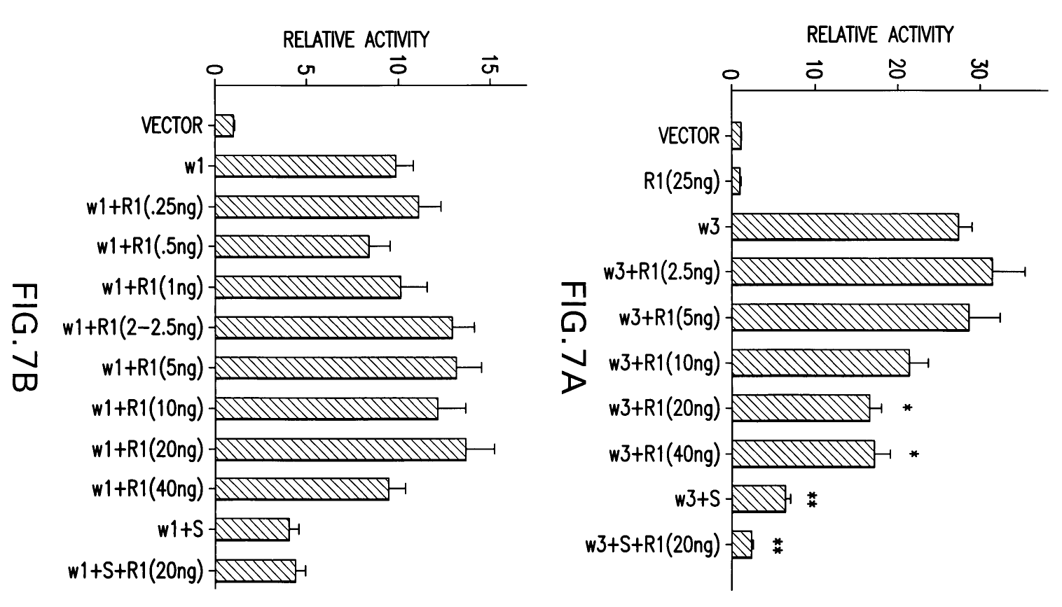

To assess the effect of Ror1 on Wnt pathway activity, a luciferase reporter assay was performed as described in Example 4. As shown in FIG. 7A, Ror1-flag had no activity on the TCF promoter by itself, but inhibited signaling by Wnt-3-HA, with the IC50=5 ng/well and the maximum inhibition of 37%. Addition of Ror1-flag at any dose examined had no effect on Wnt-1-HA-stimulated transcription (FIG. 7B).

### Example 6

### The Tyrosine Kinase Activity of Ror2 is Required for Potentiation of Wnt-1 and for Most of Inhibition of Wnt-3 Activity

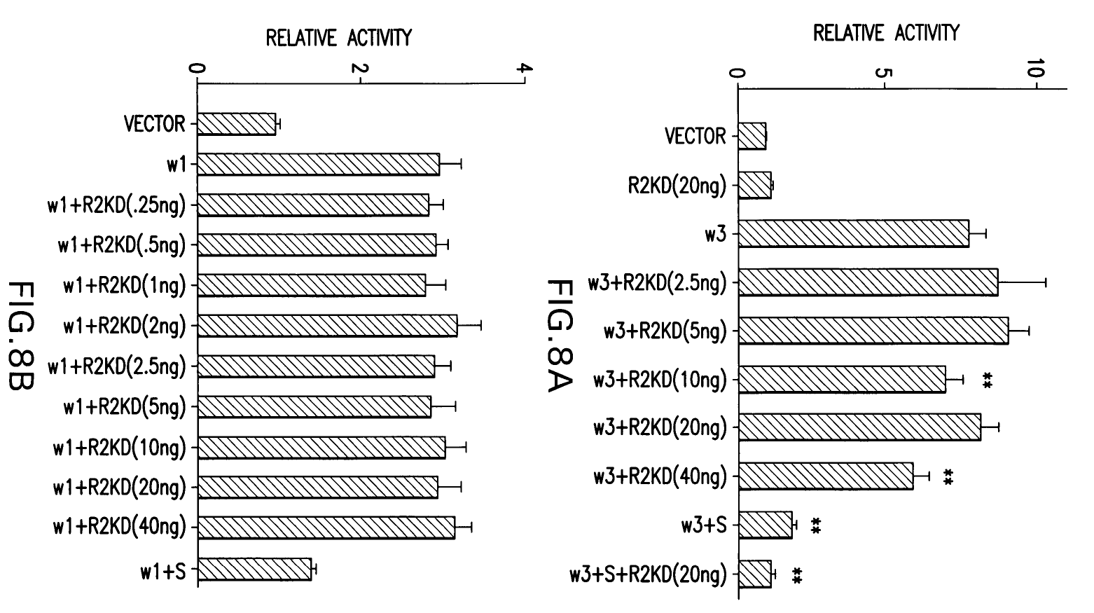

To investigate if Ror2 kinase activity is required for modulation of Wnt signaling, a luciferase reporter assay was performed as described in Example 4 using the Ror2KD point mutant that has no tyrosine kinase activity (see FIG. 5C). This mutant did not potentiate Wnt-1 signaling to the TCF promoter (FIG. 8A) and only weakly inhibited Wnt-3-induced activation of the promoter (FIG. 8B).

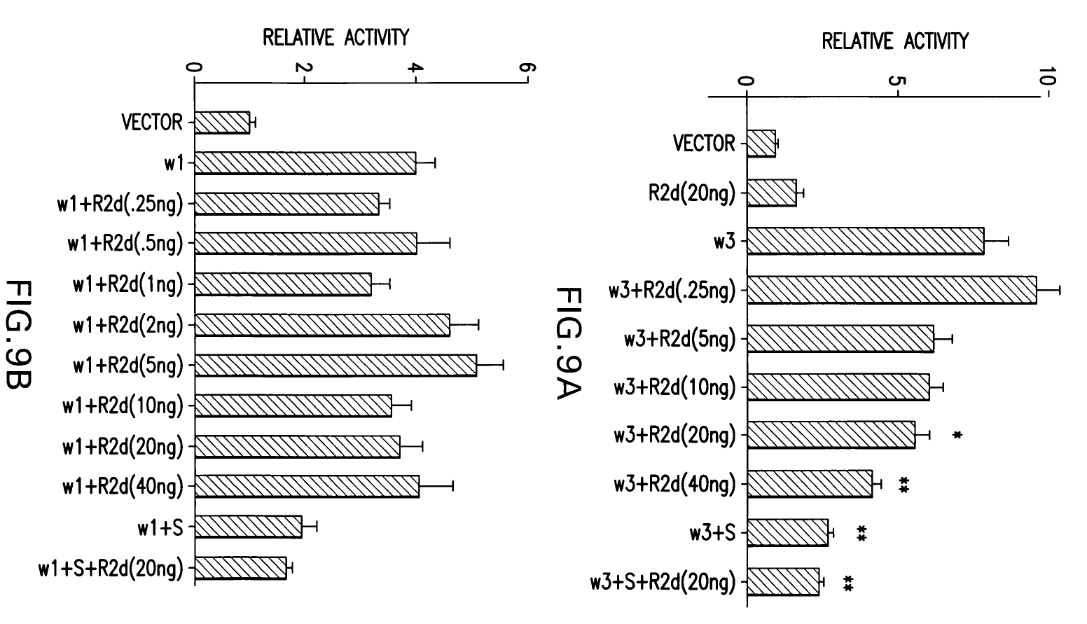

Similar results were obtained using the Ror2 mutant that lacked the entire cytoplasmic domain (Ror2ΔC-flag). Ror2ΔC-flag did not potentiate Wnt-1-HA activity (FIG. 9B) and also lost part of its ability to inhibit Wnt-3 signaling in U2OS cells (FIG. 9A). Thus, the tyrosine kinase activity of Ror2 is required for potentiation of Wnt-1 signaling and for the majority of inhibition of the Wnt-3 activity.

### Example 7

### Ror2 and Ror2KD Bind to Wnt-1 and Wnt-3 Ror2 Discriminates Between Different Wnts

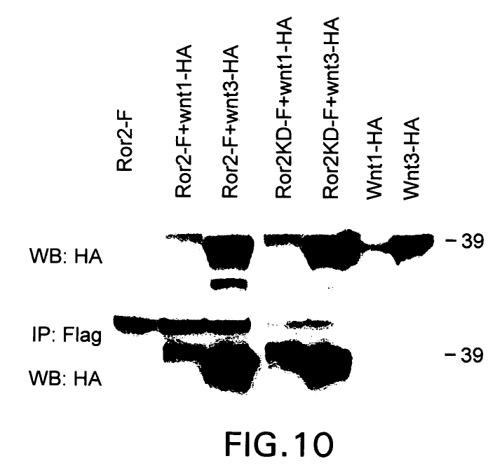

To assess possible direct binding of Ror2 to Wnts, immunoprecipitation experiments with lysates of COS-7 or U2OS cells co-transfected with Ror2-flag and HA-tagged Wnts 1 and 3 was performed as described in General Methods. FIG. 10 shows that both Wnt-1 and Wnt-3 were immunoprecipitated in complexes with Ror2. The specificity of interactions was demonstrated by the fact that anti-flag failed to immunoprecipitate Wnts in the absence of Ror2-flag co-expression. Loss of kinase activity did not affect the ability of Ror2 to bind Wnts as both Wnt-1 and Wnt-3 were immunoprecipitated in complexes with Ror2KD (FIG. 10).

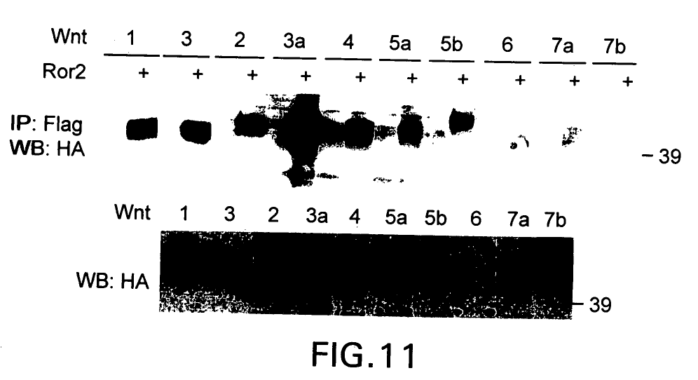

To address the specificity of Ror2-Wnt interactions, an immunoprecipitation experiment with a panel of different Wnt proteins (FIG. 11) was performed. In this experiment, ten different HA-tagged Wnts were over-expressed in COS7 cells in absence or presence of Ror2. As is shown in FIG. 11, Ror2 was observed to best bind to Wnt3a, followed by Wnt3, 4, 2, 1, 5b and 5a. Ror2 bound Wnt6 and 7a only weakly, although Wnt6 expression in COS7 cells was not as high as that of the other Wnts (FIG. 11, bottom panel). Ror2 did not bind Wnt7b under our experimental conditions. Thus, Ror2 shows specificity in its interactions with Wnts.

### Example 8

### Overexpression of Wnt-1 and Wnt-3 has No Effect on Ror2 Autophosphoryltaion

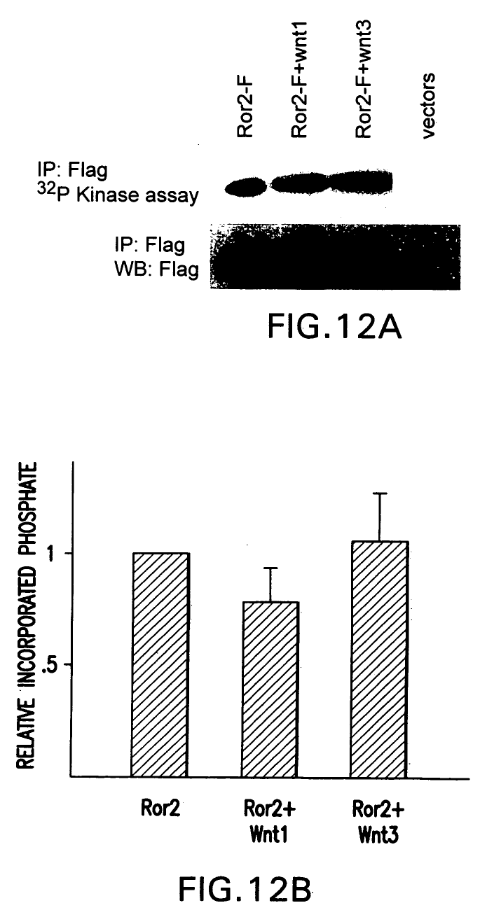

When Ror2 was immunoprecipitated out of U2OS extracts, it had ability to phosphorylate itself in vitro (see FIG. 5C), presumably due to its intrinsic tyrosine kinase activity. In order to determine whether co-expression of Wnts affects the extent of Ror2 autophosphorylation, U2OS cells were transiently transfected with Ror2 in presence of Wnt-1, Wnt-3 or a control plasmid. The whole-cell extracts were isolated 24 h later. Ror2-flag was immunoprecipitated on flag affinity agarose and in vitro autophosphorylation assay was performed as described in General Methods. FIG. 12A shows an exemplary autoradiograph followed by western immunoblot of the same membrane. In FIG. 12B, results of at least three independent experiments were quantitated using Quantity One 1-D Image Analysis Software (Bio-Rad, Hercules, Calif.) and the radioactive signals were normalized to the total amount of immunoreactive Ror2 present in each reaction. There was no significant difference in Ror2 autophosphorylation in absence or presence of Wnts 1 and 3.

### Example 9

### Ror2 Inhibits Wnt-Induced Stabilization of β-Catenin

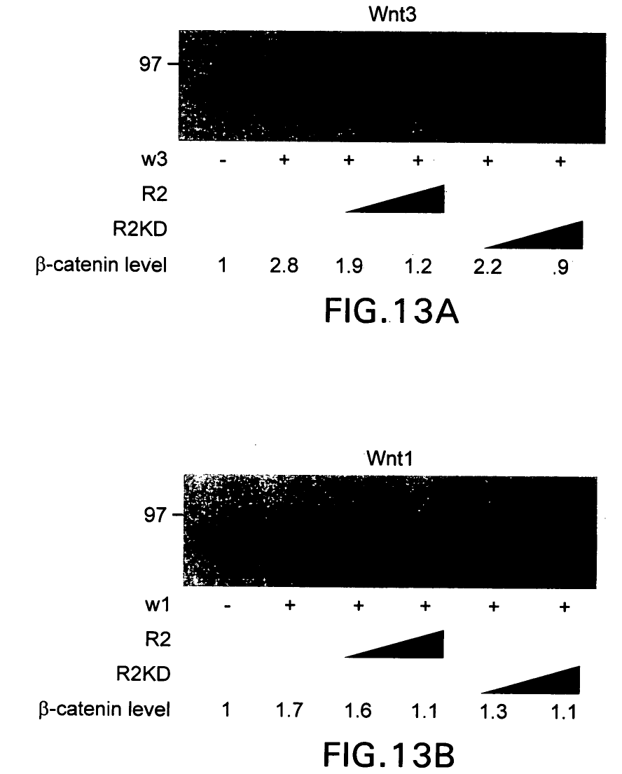

To address where in the canonical Wnt signaling pathway Ror2 functions, the ability of the Ror2 kinase to affect Wnt-mediated stabilization of cytosolic β-catenin was assessed. Upon transfection of Wnt1 or 3 into U2OS cells, the cytoplasmic levels of β-catenin reproducibly increased by 1.7- or 2.8-fold, respectively (FIG. 13). Co-transfection with increasing amounts of Ror2 dose-dependently inhibited the ability of both Wnt1 and Wnt3 to stabilize cytosolic β-catenin. Interestingly, the kinase-dead Ror2 mutant was as potent as Ror2 in inhibiting f-catenin stabilization, indicating that the kinase activity is not required for this effect. Thus Ror2-induced inhibition of Wnt3 signaling on the TCF promoter can be due, at least in part, to degradation of β-catenin. However, Ror2 must potentiate Wnt1 signaling on the TCF promoter by acting downstream of β-catenin stabilization. Furthermore, this other arm of Ror2 signaling has to overcome the Ror2-induced decrease in β-catenin levels.

### Example 10

### A Model for Ror2 Function in the Wnt Signaling

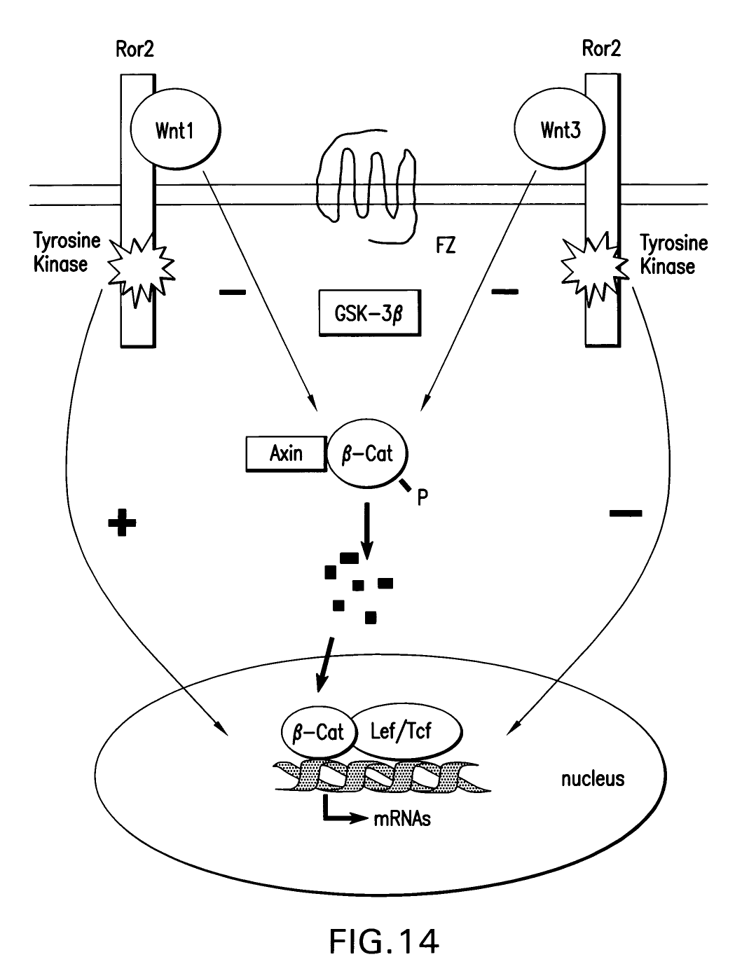

FIG. 14 presents a working model for modulation of canonical Wnt signaling by Ror2. Ror2 binds Wnt1 and Wnt3 and sequesters them away from their FZ receptors resulting in increased degradation of β-catenin. This process does not require the tyrosine kinase activity of the Ror2 receptor. In addition, Wnt1 binding to Ror2 activates a Ror2 kinase-dependent signaling pathway that ultimately results in potentiation of TCF promoter activity. The fact that Wnt binding does not appear to modulate the Ror2 kinase activity suggests that Wnt1 initiates some other event necessary for Ror2 activation, i.e. receptor dimerization, and that the background level of Ror2 autophosphorylation is sufficient for function. Wnt3 binding does not appear to activate this additional signaling pathway and Ror2 inhibits Wnt3 activity on the TCF promoter. However, additional kinase-dependent events downstream of β-catenin are required for this inhibition since kinase-dead Ror2 antagonizes □-catenin stabilization, but loses its ability to inhibit the TCF promoter. Thus the net effect of Ror2 on Wnt signaling is determined by the particular Wnts expressed in the cell.

### Example 11

### Identification of the Ror2 Binding Partners in U2OS Cells

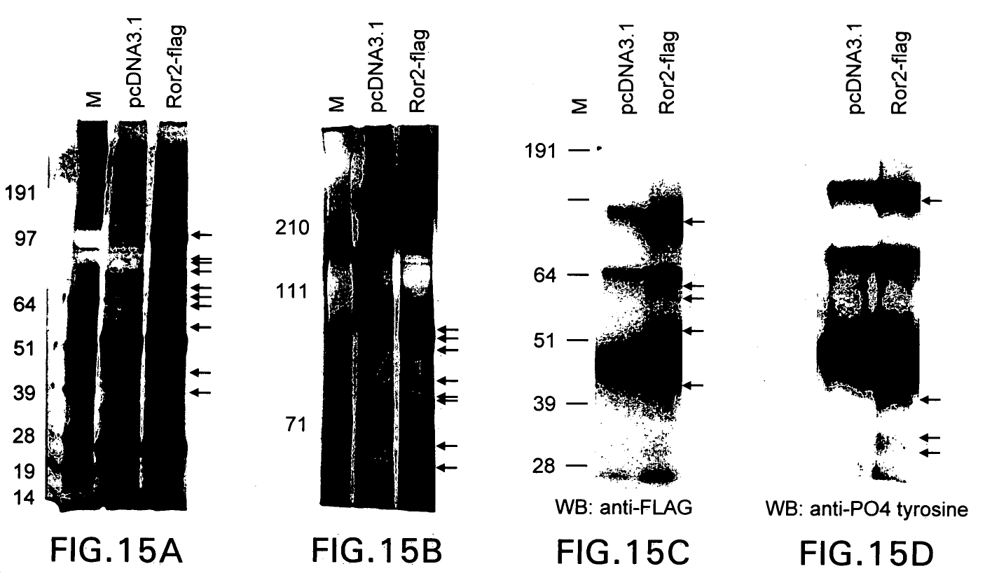

Ror2 binding partners were identified by immunoprecipitation followed by mass-spectroscopy analysis. To this end, the protein extracts of the U2OS cells transiently transfected with pcDNA3.1(+) or Ror2-flag were first immunoprecipitated on anti-flag affinity agarose and separated on different percentage SDS-PAGE gels. Silver staining identified several bands that were only present in the Ror2-flag-containing extracts (marked by arrows in FIG. 15A-C), including presumably Ror2-flag itself around 100 kDa. Importantly, several of the bands marked by arrows were not recognized by anti-flag antibody (compare FIGS. 15A and D), indicating that these proteins are distinct from Ror2-flag or its proteolytic fragments. Furthermore, some of these proteins were identified by anti-phosphotyrosine antibody (FIG. 15E). The mass-spectroscopy (MS) analysis was performed on immnuoprecipitated Ror2-flag and control samples as follows. SDS was added to the samples eluted from the immunoprecipitation resin to 0.05% (w/v). The samples were then dialized against 0.05% SDS and the volumes were reduced by evaporation to about 1/50th the original volume. The samples were then applied to a 10% tricine gel (Invitrogen) and the gel was silver stained. Slices were excised from the gel, spanning the entire lane of each sample. The proteins within each gel slice were reduced with DTT, alkylated with iodoacetamide, and subjected to in gel digestion with trypsin using a ProGest Investigator (Promega). The volume of the digested samples was reduced by evaporation and then brought back up to about 20 μl, to include 0.1M acetic acid and 2% acetonitrile.

Ten microliters of each sample were then loaded onto a 10 cm×75 μm C₁₈ reverse phase column packed in a Picofrit needle (New Objectives; Woburn, Mass.) which was in line with an LCQ Deca XP plus mass spectrometer (Finnigan; San Jose, Calif.). The peptide masses were recorded by scanning an m/z range from 375 to 1200. The fragment ion spectra (tandem mass-spectroscopy (MS/MS)) were acquired in a data-dependant manner in which each MS scan was followed by consecutive MS/MS experiments on the first three most intense ions from the MS scan. The resulting MS/MS data were searched against the NCBI nonredundant data base using the Sequest program (Finnigan).

The proteins found to be specifically immunoprecipitated by Ror2-flag are listed in Table 4.

TABLE 4
Potential Ror2 interacting proteins

| Potential Ror2 interacting proteins | Accession Number |
|---|---|
| ADP/ATP carrier protein (adenine nucleotide | AAB96347.1 |
| translocator 2) | |
| UDP-glucose ceramide glucosyltransferase-like 1 | AAH41098.1 |
| 14-3-3 protein beta/alpha | NP_003395.1 |
| 14-3-3 protein gamma | AAD48408.1 |
| ribophorin I | NP_002941.1 |
| arginine N-methyltransferase 1 | NP_062804.1 |
| cellular apoptosis susceptibility protein | AAC35008.1 |
| NOTCH2 protein | AAG37073.1 |
| HUMAN Skeletal muscle LIM-protein 3 (SLIM 3) | Q14192 |

### Example 12

### Ror2 Interacts with the Intracellular Domain of the Notch2 Receptor

In order to confirm the interaction between Ror2 and Notch2, a construct containing the intracellular domain of human Notch2 (Notch2IC, bp 5125-7431) tagged with COOH terminal V5 and his tags was generated as described in General Methods. Notch2IC-V5-His and Ror2-Flag were co-transfected into U2OS cells, and 1 mg of total cell lysates was immunoprecipitated on M2 flag affinity agarose and resolved on SDS-PAGE as described in General Methods.

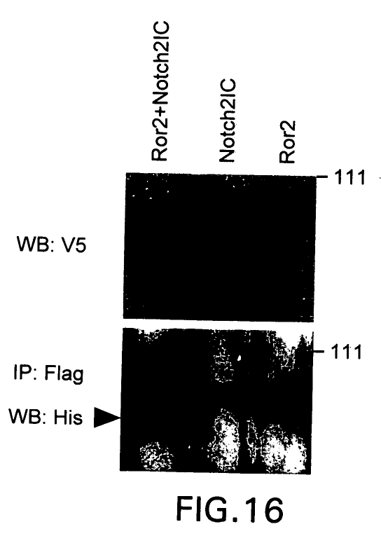

FIG. 16 shows that Notch2IC was immunoprecipitated in complexes with Ror2. The specificity of interactions was demonstrated by the fact that anti-flag failed to immunoprecipitate Notch2IC in the absence of Ror2-flag co-expression.

### Example 13

### Generation of HOB-01-09 Pre-Osteocytic Cells Stably Over-Expressing Ror2 and Ror1

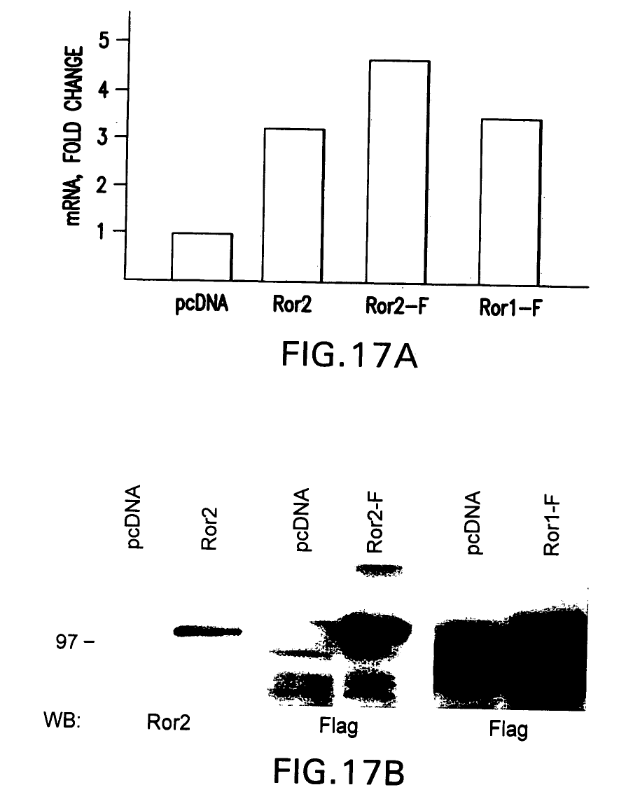

To investigate effects of Ror kinases on osteoblast physiology, we stably over-expressed Ror2, Ror2-Flag, and Ror1-Flag in one of our human osteoblastic cell lines. We used HOB-01-09 pre-osteocytes that have low levels of endogenous Ror2 (similar to HOB-01-C1 pre-osteocytes in FIG. 1). HOB-01-09 cells have been previously described (Bodine, P. V. N. (Wyeth Corp.) Composition comprising a secreted frizzled related protein—useful for the treatment of e.g. osteoarthritis. PCT# WO200119855-A2; 2000) and deposited with ATCC. Ror2, Ror2-Flag, and Ror1-Flag in pcDNA3.1(+) or empty pcDNA3.1(+) were transfected into HOB-01-09 cells by electroporation. For each transfection, 10 □g of plasmid DNA was added to 8×10⁶ cells in PBS and incubated on ice for 5 min. The mixture was electroporated in 2 mm gap cuvettes (BTX, San Diego, Calif.) using ECM 600 electroporator (BTX) with the following parameters: 100 □F, 48 Ohms, 150 V (pulse duration ˜10 msec). Cells were cooled 5 min on ice, transferred to a 150 mm culture flask and maintained at 34° C. in a 5% CO₂/95% humidified air incubator using DMEM/F-12 medium containing 10% heat-inactivated fetal bovine serum, 1% penicillin-streptomycin, and 2 mM glutaMAX-1 (HOB growth medium) supplemented with 500 μg/ml G418 (Invitrogen) until isolated colonies of G418-resistant cells were formed. Colonies were trypsinized and transferred one per well onto 96-well plates. Colonies were grown at 34° C. in HOB growth medium supplemented with 125 μg/ml G418 and levels of Ror expression were assessed by real-time RT-PCR and western immunoblotting. This procedure yielded several cell lines with 3-5-fold over-expression of Ror2, Ror2-Flag and Ror1-Flag mRNA when compared to HOB-01-09 cells over-expressing the empty vector (FIG. 17A). These lines also demonstrated expression of immunoreactive proteins of appropriate size (FIG. 17B). We conclude that these cell lines over-express Ror2 and Ror1 and can be used to assess effects of Ror kinases on osteoblastic cell phenotype, including effects on apoptosis, alkaline phosphatase activity, and osteocalcin secretion.

### Example 14

### Validation of Ror2 as an Osteoporotic Drug Target

Ror2 is validated as an osteoporotic drug target by in vitro and in vivo approaches. The siRNA molecules are generated that specifically disrupt Ror2 expression in mammalian cells. siRNAs are overexpressed in early osteoblasts with relatively high levels of Ror2 mRNA and the effects of Ror2 disruption on cell differentiation and/or survival are monitored. Gene chip analysis is performed to identify genes regulated by Ror2 expression in osteoblastic cells. Based on Ror2 expression patterns and its relationship to SFRP-1 and Wnt signaling, Ror2 down-regulation increases the osteoblast differentiation and also promotes apoptosis. A complemetary approach involves overexpression of Ror2-flag in pre-osteocytic cells that have no detectable levels of Ror2 mRNA and monitoring the differentiation state of these cells. Following in vitro validation, in vivo validation of Ror2, as an osteoporotic target, is performed by generating transgenic mice conditionally overexpressing Ror2 in a tissue- and/or time-dependent manner as well as by generating conditional Ror2 knockout mice.

### Example 15

### High Throughput Screening to Identify Ror2 Regulators

Ror2 phosphorylation is utilized as a basis for the high throughput screen (HTS) to identify small molecule agonists or antagonists. Whether to seek activators or inhibitors of Ror2 is determined by the findings of in vitro target validation and receptor agonists are screened. The general mechanism of activation of receptor tyrosine kinases is thought to be through receptor oligomerization and autophosphorylation (see ref., Forrester, W. C., Cellular & Molecular Life Sciences, 59, 83-96, (2002), and references therein) and therefore molecules modulating phosphorylation of the receptor can be expected to modulate its activity. An expression plasmid for human Ror2 containing a COOH-terminal GST tag is generated and the protein in bacterial cells is produced and is bound to gluthatione-coated multi-well plates. After unbound proteins are washed away, the level of Ror2 phosphorylation on tyrosine is determined by, for example, anti-phosphotyrosine antibody followed by detection with chemiluminescent reagents. The compound libraries is then screened in presence of ATP and divalent cations for molecules that enhance the level of Ror2 phosphorylation. This is a cell-free HTS that is generally more robust and easier to set up than a cell-based screen, but can produce ‘false positives’, i.e. compounds that will not work in intact cells. Alternatively, other cell-based as well as non-cell-based approaches can be used to set up the HTS. These include, but are not limited to, screening the compounds for their potential to antagonize the ability of Ror2 to disrupt signaling by Wnt-3 in the TCF promoter-luciferase reporter assay.

### Example 16

### Validation of Compositions that Affect Ror2 Kinase Activity

Compounds that modified Ror2 kinase activity in the HTS, is then move on to additional in vitro assays. The first bench-top confirmation assay includes overexpression of Ror2 in a mammalian cell line and treatment with candidate compounds followed by western immunoblotting with anti-phosphotyrosine antibody. This assay is also used to determine the potency and efficacy of the confirmed compounds for modulating Ror2 kinase activity. Additional assays are designed to measure the ability of these compounds to block human osteoblastic cell death or promote osteoblastic differentiation in a Ror2-dependent or independent manner and to determine the potency and efficacy of these compounds for these effects. Additional assays are also used to determine the cell selectivity of these compounds (e.g., by using HeLa or other Ror2-expressing cell lines), as well as the specificity of these compounds for Ror2 versus another family member, Ror1. Additional assays are also employed to determine whether these compounds regulate down-stream signaling events involved in apoptosis (e.g., caspase activity), differentiation (e.g., alkaline phosphatase activity or osteocalcin expression), or Wnt activity (e.g., β-catenin levels and function via the TCF-luciferase assay). Finally, compounds that exhibited appropriate activities in these in vitro assays can be used in a variety of animal models for bone formation, osteopenia, or osteoporosis (e.g., ovariectomized rats or mice). A compound that inhibited osteoblast/osteocyte apoptosis or promoted osteoblast differentiation would conceivably be an anabolic bone agent by prolonging the lives of these cells and thereby either increasing the amount of bone matrix that is synthesized and mineralized and/or maintaining the integrity of the bone.

### Example 17

### High Throughput Screening to Identify Ror2 Regulators

Ror2 phosphorylation is utilized as a basis for the high throughput screen (HTS) to identify small molecule agonists or antagonists. Whether to seek activators or inhibitors of Ror2 is determined by the findings of in vitro target validation and receptor agonists are screened. The general mechanism of activation of receptor tyrosine kinases is thought to be through receptor oligomerization and autophosphorylation (see ref., Forrester, W. C., Cellular & Molecular Life Sciences, 59, 83-96, (2002), and references therein) and therefore molecules modulating phosphorylation of the receptor can be expected to modulate its activity. An expression plasmid for human Ror2 containing a COOH-terminal GST tag is generated and the protein in bacterial cells is produced and is bound to gluthatione-coated multi-well plates. After unbound proteins are washed away, the level of Ror2 phosphorylation on tyrosine is determined by, for example, anti-phosphotyrosine antibody followed by detection with chemiluminescent reagents. The compound libraries is then screened in presence of ATP and divalent cations for molecules that enhance the level of Ror2 phosphorylation. This is a cell-free HTS that is generally more robust and easier to set up than a cell-based screen, but can produce ‘false positives’, i.e. compounds that will not work in intact cells. Alternatively, other cell-based as well as non-cell-based approaches can be used to set up the HTS. These include, but are not limited to, screening the compounds for their potential to antagonize the ability of Ror2 to disrupt signaling by Wnt-3 in the TCF promoter-luciferase reporter assay.

### Example 18

### Validation of Compositions that Affect Ror2 Kinase Activity

Compounds that modified Ror2 kinase activity in the HTS, is then move on to additional in vitro assays. The first bench-top confirmation assay includes overexpression of Ror2 in a mammalian cell line and treatment with candidate compounds followed by western immunoblotting with anti-phosphotyrosine antibody. This assay is also used to determine the potency and efficacy of the confirmed compounds for modulating Ror2 kinase activity. Additional assays are designed to measure the ability of these compounds to block human osteoblastic cell death or promote osteoblastic differentiation in a Ror2-dependent or independent manner and to determine the potency and efficacy of these compounds for these effects. Additional assays are also used to determine the cell selectivity of these compounds (e.g., by using HeLa or other Ror2-expressing cell lines), as well as the specificity of these compounds for Ror2 versus another family member, Ror1. Additional assays are also employed to determine whether these compounds regulate down-stream signaling events involved in apoptosis (e.g., caspase activity), differentiation (e.g., alkaline phosphatase activity or osteocalcin expression), or Wnt activity (e.g., β-catenin levels and function via the TCF-luciferase assay). Finally, compounds that exhibited appropriate activities in these in vitro assays can be used in a variety of animal models for bone formation, osteopenia, or osteoporosis (e.g., ovariectomized rats or mice). A compound that inhibited osteoblast/osteocyte apoptosis or promoted osteoblast differentiation would conceivably be an anabolic bone agent by prolonging the lives of these cells and thereby either increasing the amount of bone matrix that is synthesized and mineralized and/or maintaining the integrity of the bone.

1. An expression cassette comprising a polynucleoticle encoding a Ror polypeptide or homologues or derivatives or fragments or variants or mutants thereof wherein said polynucleotide is under the control of a promoter operable in bone cells.

2. The expression cassette of claim 1, wherein Ror polypeptide is Ror1 polypeptide.

3. The expression cassette of claim 1, wherein Ror polypeptide is Ror2 polypeptide.

4. The expression cassette of claim 1, wherein said promoter is heterologous to the coding sequence.

5. The expression cassette of claim 1, wherein the promoter is a bone-specific promoter.

6. The expression cassette of claim 5, wherein the bone-specific promoter is a rat 3.6 kb type I collagen or rat 1.7 kb osteocalcin promoter.

7. The expression cassette of claim 1, wherein said promoter is an inducible promoter.

8. A vector comprising the expression cassette of claim 1.

9. The vector of claim 8, wherein the vector is a viral vector.

10. The vector of claim 9, wherein said viral vector is selected from the group consisting of a retroviral vector, an adenoviral vector, an adeno-associated viral vector, a vaccinia viral vector, and a herpes viral vector.

11. The expression cassette of claim 1, wherein said expression cassette further comprises a polyadenylation signal.

12. A host cell comprising an expression cassette comprising a polynucleotide encoding a Ror polypeptide or homologues or derivatives or fragments or variants or mutants thereof, wherein said polynucleotide is under the control of a promoter operable in eukaryotic cells, said promoter being heterologous to said polynucleotide.

13. The host cell of claim 12, wherein Ror polypeptide is Ror1 polypeptide.

14. The host cell of claim 12, wherein Ror polypeptide is Ror2 polypeptide.

15. A composition for modulating bone-related activity comprising an effective amount of Ror molecule or homologues or derivatives or fragments or variants or mutants thereof.

16. The composition of claim 15, wherein Ror molecule is Ror1 molecule.

17. The composition of claim 15, wherein Ror molecule is Ror2 molecule.

18. The composition of claim 15, further comprising a pharmaceutically acceptable carrier.

19. The composition of claim 15, wherein bone-related activity is osteoblast differentiation, osteoclast differentiation, osteoblast survival, osteoclast survival, osteoblast activity, or osteoclast activity.

20. A method of screening for agents, the method comprising: (a) combining an agent with a Ror molecule; and (b) detecting an effect of said agent on Ror activity; wherein detection of a decrease or an increase in Ror activity is indicative of an agent being a bone-related agent.

21. The method of claim 20, wherein a decrease or an increase in Ror activity is detected by a decrease or an increase in Ror-induced inhibition of Wnt-3 signaling.

22. The method of claims 20, wherein Ror molecule is Ror1 molecule and Ror activity is Ror1 activity.

23. The method of claims 20, wherein Ror molecule is Ror2 molecule and Ror activity is Ror2 activity.

24. The method of claim 20, wherein Ror molecule is Ror2 molecule and a decrease or an increase in Ror activity is detected by a decrease or an increase in Ror2-induced activation of Wnt-1 signaling.

25. The method of claim 20, wherein Ror molecule is a Ror polypeptide and a decrease or an increase in Ror activity is detected by a decrease or an increase in Ror autophosphorylation.

26. The method of claim 25, wherein Ror polypeptide is Ror1 polypeptide, and Ror activity is Ror1 activity.

27. The method of claim 25, wherein Ror polypeptide is Ror2 polypeptide, and Ror activity is Ror2 activity.

28. A method of screening for agents, the method comprising: (a) combining an agent with an isolated cell comprising a Ror promoter sequence operatively linked to a reporter gene; and (b) detecting an effect of said agent on reporter activity; wherein detection of a decrease or an increase in Ror promoter activity as measured by the reporter activity is indicative of an agent being a bone-related agent.

29. The method of claim 28, wherein Ror promoter is Ror1 promoter.

30. The method of claim 29, wherein Ror1 promoter is human Ror1 promoter.

31. The method of claim 28, wherein Ror promoter is Ror2 promoter.

32. The method of claim 31, wherein Ror2 promoter is mouse Ror2 promoter.

33. A method of screening for agents that modulate the binding of Ror polypeptide to a binding partner comprising: (a) contacting Ror polypeptide with a Ror binding partner in the presence of an agent; (b) contacting Ror polypeptide with a Ror binding partner in the presence of a control or in the absence of the agent; and (c) selecting the agent that modulates Ror polypeptide binding to Ror binding partner by comparing the binding of said Ror polypeptide to the binding partner in step (a) to the binding of said Ror polypeptide to a binding partner in step (b).

34. The method of claim 33, wherein Ror polypeptide is Ror2 polypeptide and Ror binding partner is Ror2 binding partner.

35. The method of claim 34, wherein Ror2 binding partner is selected from the group consisting of ADP/ATP carrier protein, UDP-glucose ceramide glucosyltransferase-like 1, 14-3-3 protein beta/alpha, 14-3-3 protein gamma, ribophorin I, arginine N-methyltransferase 1, cellular apoptosis susceptibility protein, NOTCH2 protein, and human skeletal muscle LIM-protein 3.

36. A method of modulating bone-related activity in a subject comprising administering to a subject an agent which modulates target Ror molecule expression or activity.

37. The method of claim 36, wherein said agent comprises one or more of Ror molecules or homologues or derivatives or fragments or variants or mutants thereof.

38. The method of claim 36, wherein said agent comprises one or more of Ror molecule binding partners or homologues or derivatives or fragments or variants or mutants thereof.

39. The method of claims 36, wherein target Ror molecule is Ror1 molecule.

40. The method of claims 36, wherein target Ror molecule is Ror2 molecule.

41. The method of claim 36, wherein target Ror molecule activity is tyrosine kinase activity.

42. The method of claim 36, wherein bone-related activity is osteoblast differentiation, osteoclast differentiation, osteoblast survival, osteoclast survival, osteoblast activity, or osteoclast activity.

43. The method of claim 36, wherein said agent is selected from the group consisting of an antibody, a small molecule, a peptide, an oligopeptide, and a polypeptide.

44. The method of claim 36, wherein said agent comprises an antisense nucleic acid or siRNA molecule specific for Ror gene and wherein antisense nucleic acid or siRNA molecule recognize and bind to a nucleic acid encoding one or more Ror polypetides or homologues or derivatives or fragments or variants or mutants thereof.

45. The method of claim 44, wherein Ror gene is Ror1 gene and Ror polypeptide is Ror1 polypeptide.

46. The method of claim 44, wherein Ror gene is Ror2 gene and Ror polypeptide is Ror2 polypeptide.

47. The method of claim 36, wherein said agent modulates expression and/or activity of target Ror molecules or homologues or derivatives or fragments or variants or mutants thereof by binding to Ror binding partners.

48. The method of claim 47, wherein said agent inhibits expression and/or activity of target Ror molecules or homologues or derivatives or fragments or variants or mutants thereof by binding to Ror binding partners.

49. The method of claim 47, wherein said agent enhances expression and/or activity of target Ror molecules or homologues or derivatives or fragments or variants or mutants by binding to Ror binding partners.

50. The method as in any one of claims 47-49, wherein target Ror molecule is Ror2 molecule and Ror binding partner is Ror2 binding partner.

51. The method of claim 50, wherein Ror2 binding partners are selected from the group consisting of ADP/ATP carrier protein, UDP-glucose ceramide glucosyltransferase-like 1, 14-3-3 protein beta/alpha, 14-3-3 protein gamma, ribophorin I, arginine N-methyltransferase 1, cellular apoptosis susceptibility protein, NOTCH2 protein, and human skeletal muscle LIM-protein 3.

52. The method of claim 36, wherein said agent modulates binding of Ror molecules or homologues or derivatives or fragments or variants or mutants thereof to Ror binding partners.

53. The method of claim 52, wherein said agent enhances binding of Ror molecules or homologues or derivatives or fragments or variants or mutants thereof to Ror binding partners.

54. The method of claim 52, wherein said agent inhibits binding of Ror moelcules or homologues or derivatives or fragments or variants or mutants thereof to Ror binding partners.

55. The method as in any one of claims 52, wherein Ror molecule is Ror2 molecule and Ror binding partner is Ror2 binding partner.

56. The method of claim 55, wherein Ror2 binding partners are selected from the group consisting of ADP/ATP carrier protein, UDP-glucose ceramide glucosyltransferase-like 1, 14-3-3 protein beta/alpha, 14-3-3 protein gamma, ribophorin I, arginine N-methyltransferase 1, cellular apoptosis susceptibility protein, NOTCH2 protein, and human skeletal muscle LIM-protein 3.

57. The method of claim 36, wherein the agent is administered to isolated cells in culture.

58. The method of claim 57, wherein the cells are primary osteoblasts, immortalized cell lines of osteoblastic origin, or immortalized cell lines of non-osteoblastic origin.

59. The method of claim 58, wherein immortalized cell lines of osteoblastic origin are selected from the group consisting of HOB, U2OS, and SaOS-2 cells.

60. The method of claim 36, wherein the agent is administered to a non-human transgenic animal.

61. The method of claim 60, wherein, the transgenic animal is a mouse.

62. The method of claim 60, wherein the agent is administered to a non-human knockout animal.

63. The method of claim 36, wherein said subject is a vertebrate or an invertebrate organism.

64. The method of claim 36, wherein said subject is a mammal.

65. The method of claim 64, wherein said mammal is a human.

66. A method of modulating Wnt-1 and Wnt-3 activity in a subject comprising administering an agent which modulates target Ror2 molecule expression or activity in an amount effective to regulate Wnt-1 and Wnt-3 activity.

67. A method for identifying an agent for modulating bone-related activity comprising: (a) expressing Ror molecule in a cell or using endogenous Ror expression; (b) contacting the cell with the agent; and (c) monitoring the expression or the activity of Ror molecule wherein an increase or decrease in the expression or activity of Ror molecule in the presence of the agent identifies the agent as modulating bone-related activity.

68. The method of claim 67, wherein Ror molecule is Ror1 molecule.

69. The method of claim 67, wherein Ror molecule is Ror2 molecule.

70. The method of claim 67, wherein Ror molecule activity is tyrosine kinase activity.

71. The method of claim 67, wherein bone-related activity is osteoblast differentiation, osteoclast differentiation, osteoblast survival, osteoclast survival, osteoblast activity, or osteoclast activity.

72. The method of claim 67, wherein said agent is selected from the group consisting of antibody, small molecule, peptide, oligopeptide, ribozyme, and polypeptide.

73. The method of claim 67, wherein said agent comprises an antisense nucleic acid or siRNA molecule specific for Ror gene and wherein antisense nucleic acid or siRNA molecule recognize and bind to a nucleic acid encoding one or more Ror polypetides or homologues or derivatives or fragments or variants or mutants thereof.

74. The method of claim 73, wherein Ror gene is Ror1 gene and Ror polypeptide is Ror1 polypeptide.

75. The method of claim 73, wherein Ror gene is Ror2 gene and Ror polypeptide is Ror2 polypeptide.

76. The method of claim 67, wherein said agent modulates expression and/or activity of Ror molecules or homologues or derivatives or fragments or variants or mutants thereof by binding to Ror binding partners.

77. The method of claims 76, wherein Ror molecule is Ror2 molecule and Ror binding partner is Ror2 binding partner.

78. The method of claim 77, wherein Ror2 binding partners are selected from the group consisting of ADP/ATP carrier protein, UDP-glucose ceramide glucosyltransferase-like 1, 14-3-3 protein beta/alpha, 14-3-3 protein gamma, ribophorin I, arginine N-methyltransferase 1, cellular apoptosis susceptibility protein, NOTCH2 protein, and human skeletal muscle LIM-protein 3.

79. The method of claim 67, wherein said agent modulates binding of Ror molecules or homologues or derivatives or fragments or variants or mutants thereof to Ror binding partners.

80. The method of claims 79, wherein Ror molecule is Ror2 molecule and Ror binding partner is Ror2 binding partner.

81. The method of claim 67, wherein the cells are primary osteoblasts, immortalized cell lines of osteoblastic origin, or immortalized cell lines of non-osteoblastic origin

82. The method of claim 81, wherein immortalized cell lines of osteoblastic origin are selected from the group consisting of HOB, U2OS, and SaOS-2 cells.

83. The method of claim 67, wherein the agent is further administered to a vertebrate organism to monitor the expression or the activity of Ror molecule wherein an increase or decrease in the expression or activity of Ror molecule in the presence of the agent identifies the agent as modulating bone-related activity.

84. The method of claim 83, wherein said vertebrate organism is a mammal.

85. The method of claim 83, wherein said vertebrate organism is a non-human transgenic animal.

86. A method for identifying an agent for modulating Wnt signaling pathway comprising: screening one or more agents for the ability to modulate expression or activity of Ror molecule, wherein the agent that can modulate expression or activity of Ror molecule is an agent that modulates Wnt signaling pathway.

87. A method of linking a bioactive molecule to a cell expressing a Wnt polypeptide, said method comprising contacting a cell with a Ror2 polypeptide that is bound to a bioactive molecule and allowing a Wnt polypeptide and said Ror2 polypeptide to bind to one another, thereby linking said bioactive molecule to said cell.

88. The method of claim 87, wherein Wnt polypeptide is selected from the group consisting of Wnt-1 and Wnt-3.

89. A method for screening a subject for a bone-related disorder comprising the steps of: measuring the expression of Ror molecule in a subject and determining the relative expression of said Ror molecule in the subject compared to its expression in normal subjects, or compared to its expression in the same subject after being treated for bone-related disorders.

90. A method of identifying genes that participate in bone formation comprising: a) overexpressing Ror molecule in a cell, b) monitoring the changes in gene expression profile and c) determining which genes are regulated by Ror expression thereby identifying genes that participate in bone formation.

91. A method of identifying genes that modulate Wnt signaling pathway comprising: a) overexpressing Ror molecule in a cell, b) monitoring the changes in gene expression profile and c) determining which genes are regulated by Ror expression thereby identifying genes that modulate Wnt signaling pathway.

92. A method for identifying proliferating human pre-osteoblastic cells using Ror2 as a marker, comprising determining expression of Ror2 gene in a human osteoblastic cell wherein the increased Ror2 expression identifies the cell as being proliferating pre-osteoblastic cells.


SEQUENCE LISTING (48 sequences)

SEQ ID NO: 1 - DNA, 55 bp, Homo sapiens
ctagcgagaacaaaggagattcaaaggagatcaaaggagatcaaaggactagttc

SEQ ID NO: 2 - DNA, 55 bp, Homo sapiens
tcgagaactagtcctttgatctcctttgatctcctttgaatctcctttgttctcg

SEQ ID NO: 3 - DNA, 3458 bp, Homo sapiens
ggtaccggtccggaattcccgggatggagcgtggagagctggagcagccgccaccgccgccgccgagggagccccgggacggcagcccctgggcgcagggtgcgctgttctcggagtccgacccagggcgactcacgcccactggtgcgacccggacagcctgggactgacccgccggcccaggcgaggctgcagccagagggctgggaagggatcgcgctcgcggcatccagaggcggccaggcggaggcgagggagcaggttagagggacaaagagctttgcagacgtccccggcgtcctgcgagcgccagcggccgggacgaggcggccgggagcccgggaagagcccgtggatgttctgcgcgcggcctgggagccgccgccgccgccgcctcagcgagaggaggaatgcaccggccgcgccgccgcgggacgcgcccgccgctcctggcgctgctggccgcgctgctgctggccgcacgcggggctgctgcccaagaaacagagctgtcagtcagtgctgaattagtgcctacctcatcatggaacatctcaagtgaactcaacaaagattcttacctgacccttgatgaaccaatgaataacatcaccacgtctctgggccagacagcagaactgcactgcaaagtctctgggaatccacctcccaccatccgctggttcaaaaatgatgctcctgtggtccaggagccccggaggctctcctttcggtccaccatctatggctctcggctgcggattagaaacctcgacaccacagacacaggctacttccagtgcgtggcaacaaacggcaaggaggtggtttcttccactggagtcttgtttgtcaagtttggcccccctcccactgcaagtccaggatactcagatgagtatgaagaagatggattctgtcagccatacagagggattgcatgtgcaagatttattggcaaccgcaccgtctatatggagtctttgcacatgcaaggggaaatagaaaatcagatcacagctgccttcactatgattggcacttccagtcacttatctgataagtgttctcagttcgccattccttccctgtgccactatgccttcccgtactgcgatgaaacttcatccgtcccaaagccccgtgacttgtgtcgcgatgaatgtgaaatcctggagaatgtcctgtgtcaaacagagtacatttttgcaagatcaaatcccatgattctgatgaggctgaaactgccaaactgtgaagatctcccccagccagagagcccagaagctgcgaactgtatccggattggaattcccatggcagatcctataaataaaaatcacaagtgttataacagcacaggtgtggactaccgggggaccgtcagtgtgaccaaatcagggcgccagtgccagccatggaattcccagtatccccacacacacactttcaccgcccttcgtttcccagagctgaatggaggccattcctactgccgcaacccagggaatcaaaaggaagctccctggtgcttcaccttggatgaaaactttaagtctgatctgtgtgacatcccagcttgcgattcaaaggattccaaggagaagaataaaatggaaatcctgtacatactagtgccaagtgtggccattcccctggccattgctttactcttcttcttcatttgcgtctgtcggaataaccagaagtcatcgtcggcaccagtccagaggcaaccaaaacacgtcagaggtcaaaatgtggagatgtcaatgctgaatgcatataaacccaagagcaaggctaaagagctacctctttctgctgtacgctttatggaagaattgggtgagtgtgcctttggaaaaatctataaaggccatctctatctcccaggcatggaccatgctcagctggttgctatcaagaccttgaaagactataacaacccccagcaatggacggaatttcaacaagaagcctccctaatggcagaactgcaccaccccaatattgtctgccttctaggtgccgtcactcaggaacaacctgtgtgcatgctttttgagtatattaatcagggggatctccatgagttcctcatcatgagatccccacactctgatgttggctgcagcagtgatgaagatgggactgtgaaatccagcctggaccacggagattttctgcacattgcaattcagattgcagctggcatggaatacctgtctagtcacttctttgtccacaaggaccttgcagctcgcaatattttaatcggagagcaacttcatgtaaagatttcagacttggggctttccagagaaatttactccgctgattactacagggtccagagtaagtccttgctgcccattcgctggatgccccctgaagccatcatgtatggcaaattctcttctgattcagatatctggtcctttggggttgtcttgtgggagattttcagttttggactccagccatattatggattcagtaaccaggaagtgattgagatggtgagaaaacggcagctcttaccatgctctgaagactgcccacccagaatgtacagcctcatgacagagtgctggaatgagattccttctaggagaccaagatttaaagatattcacgtccggcttcggtcctgggagggactctcaagtcacacaagctctactactccttcagggggaaatgccaccacacagacaacctccctcagtgccagcccagtgagtaatctcagtaaccccagatatcctaattacatgttcccgagccagggtattacaccacagggccagattgctggtttcattggcccgccaatacctcagaaccagcgattcattcccatcaatggatacccaatacctcctggatatgcagcgtttccagctgcccactaccagccaacaggtcctcccagagtgattcagcactgcccacctcccaagagtcggtccccaagcagtgccagtgggtcgactagcactggccatgtgactagcttgccctcatcaggatccaatcaggaagcaaatattcctttactaccacacatgtcaattccaaatcatcctggtggaatgggtatcaccgtttttggcaacaaatctcaaaaaccctacagaattgactcaaagcaagcatctttactaggagacgccaatattcatggacacaccgaatctatgatttctgcagaactgtaaaatgcacaacttttgtaaatgtggtatacaggacaaactagacggccgtagaaaagatttatattcaaatgtttttattaaagtaaggttctcatttagcagacatcgcaacaagtaccttctgtgaagtttcactgtgtcttaccaagcaggacagacactcggccagaaaaaaaaaaaaaaaaaaaaaaaaagggcggccgctctagagtatccctcgaggggcccaagctt

SEQ ID NO: 4 - protein, 937 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYRIDSKQASLLGDANIHGHTESMISAEL

SEQ ID NO: 5 - DNA, 2832 bp, Homo sapiens
atggcccggggctcggcgctcccgcggcggccgctgctgtgcatcccggccgtctgggcggccgccgcgcttctgctctcagtgtcccggacttcaggtgaagtggaggttctggatccgaacgaccctttaggaccccttgatgggcaggacggcccgattccaactctgaaaggttactttctgaattttctggagccagtaaacaatatcaccattgtccaaggccagacggcaattctgcactgcaaggtggcaggaaacccaccccctaacgtgcggtggctaaagaatgatgccccggtggtgcaggagccgcggcggatcatcatccggaagacagaatatggttcacgactgcgaatccaggacctggacacgacagacactggctactaccagtgcgtggccaccaacgggatgaagaccattaccgccactggcgtcctgtttgtgcggctgggtccaacgcacagcccaaatcataactttcaggatgattaccacgaggatgggttctgccagccttaccggggaattgcctgtgcacgcttcattggcaaccggaccatttatgtggactcgcttcagatgcagggggagattgaaaaccgaatcacagcggccttcaccatgatcggcacgtctacgcacctgtcggaccagtgctcacagttcgccatcccatccttctgccacttcgtgtttcctctgtgcgacgcgcgctcccgggcacccaagccgcgtgagctgtgccgcgacgagtgcgaggtgctggagagcgacctgtgccgccaggagtacaccatcgcccgctccaacccgctcatcctcatgcggcttcagctgcccaagtgtgaggcgctgcccatgcctgagagccccgacgctgccaactgcatgcgcattggcatcccagccgagaggctgggccgctaccatcagtgctataacggctcaggcatggattacagaggaacggcaagcaccaccaagtcaggccaccagtgccagccgtgggccctgcagcacccccacagccaccacctgtccagcacagacttccctgagcttggaggggggcacgcctactgccggaaccccggaggccagatggagggcccctggtgctttacgcagaataaaaacgtacgcatggaactgtgtgacgtaccctcgtgtagtccccgagacagcagcaagatggggattctgtacatcttggtccccagcatcgcaattccactggtcatcgcttgccttttcttcttggtttgcatgtgccggaataagcagaaggcatctgcgtccacaccgcagcggcgacagctgatggcctcgcccagccaagacatggaaatgcccctcattaaccagcacaaacaggccaaactcaaagagatcagcctgtctgcggtgaggttcatggaggagctgggagaggaccggtttgggaaagtctacaaaggtcacctgttcggccctgccccgggggagcagacccaggctgtggccatcaaaacgctgaaggacaaagcggaggggcccctgcgggaggagttccggcatgaggctatgctgcgagcacggctgcaacaccccaacgtcgtctgcctgctgggcgtggtgaccaaggaccagcccctgagcatgatcttcagctactgttcgcacggcgacctccacgaattcctggtcatgcgctcgccgcactcggacgtgggcagcaccgatgatgaccgcacggtgaagtccgccctggagccccccgacttcgtgcaccttgtggcacagatcgcggcggggatggagtacctatccagccaccacgtggttcacaaggacctggccacccgcaatgtgctagtgtacgacaagctgaacgtgaagatctcagacttgggcctcttccgagaggtgtatgccgccgattactacaagctgctggggaactcgctgctgcctatccgctggatggccccagaggccatcatgtacggcaagttctccatcgactcagacatctggtcctacggtgtggtcctgtgggaggtcttcagctacggcctgcagccctactgcgggtattccaaccaggatgtggtggagatgatccggaaccggcaggtgctgccttgccccgatgactgtcccgcctgggtgtatgccctcatgatcgagtgctggaacgagttccccagccggcggccccgcttcaaggacatccacagccggctccgagcctggggcaacctttccaactacaacagctcggcgcagacctcgggggccagcaacaccacgcagaccagctccctgagcaccagcccagtgagcaatgtgagcaacgcccgctacgtggggcccaagcagaaggccccgcccttcccacagccccagttcatccccatgaagggccagatcagacccatggtgcccccgccgcagctctacatccccgtcaacggctaccagccggtgccggcctatggggcctacctgcccaacttctacccggtgcagatcccaatgcagatggccccgcagcaggtgcctcctcagatggtccccaagcccagctcacaccacagtggcagtggctccaccagcacaggctacgtcaccacggccccctccaacacatccatggcagacagggcagccctgctctcagagggcgctgatgacacacagaacgccccagaagatggggcccagagcaccgtgcaggaagcagaggaggaggaggaaggctctgtcccagagactgagctgctgggggactgtgacactctgcaggtggacgaggcccaagtccagctggaagcttga

SEQ ID NO: 6 - protein, 943 aa, Homo sapiens [sequence omitted]

SEQ ID NO: 7 - DNA, 3248 bp, Homo sapiens
ggtaccggtccggaattcccgggatggagcgtggagagctggagcagccgccaccgccgccgccgagggagccccgggacggcagcccctgggcgcagggtgcgctgttctcggagtccgacccagggcgactcacgcccactggtgcgacccggacagcctgggactgacccgccggcccaggcgaggctgcagccagagggctgggaagggatcgcgctcgcggcatccagaggcggccaggcggaggcgagggagcaggttagagggacaaagagctttgcagacgtccccggcgtcctgcgagcgccagcggccgggacgaggcggccgggagcccgggaagagcccgtggatgttctgcgcgcggcctgggagccgccgccgccgccgcctcagcgagaggaggaatgcaccggccgcgccgccgcgggacgcgcccgccgctcctggcgctgctggccgcgctgctgctggccgcacgcggggctgctgcccaagaaacagagctgtcagtcagtgctgaattagtgcctacctcatcatggaacatctcaagtgaactcaacaaagattcttacctgacccttgatgaaccaatgaataacatcaccacgtctctgggccagacagcagaactgcactgcaaagtctctgggaatccacctcccaccatccgctggttcaaaaatgatgctcctgtggtccaggagccccggaggctctcctttcggtccaccatctatggctctcggctgcggattagaaacctcgacaccacagacacaggctacttccagtgcgtggcaacaaacggcaaggaggtggtttcttccactggagtcttgtttgtcaagtttggcccccctcccactgcaagtccaggatactcagatgagtatgaagaagatggattctgtcagccatacagagggattgcatgtgcaagatttattggcaaccgcaccgtctatatggagtctttgcacatgcaaggggaaatagaaaatcagatcacagctgccttcactatgattggcacttccagtcacttatctgataagtgttctcagttcgccattccttccctgtgccactatgccttcccgtactgcgatgaaacttcatccgtcccaaagccccgtgacttgtgtcgcgatgaatgtgaaatcctggagaatgtcctgtgtcaaacagagtacatttttgcaagatcaaatcccatgattctgatgaggctgaaactgccaaactgtgaagatctcccccagccagagagcccagaagctgcgaactgtatccggattggaattcccatggcagatcctataaataaaaatcacaagtgttataacagcacaggtgtggactaccgggggaccgtcagtgtgaccaaatcagggcgccagtgccagccatggaattcccagtatccccacacacacactttcaccgcccttcgtttcccagagctgaatggaggccattcctactgccgcaacccagggaatcaaaaggaagctccctggtgcttcaccttggatgaaaactttaagtctgatctgtgtgacatcccagcttgcgattcaaaggattccaaggagaagaataaaatggaaatcctgtacatactagtgccaagtgtggccattcccctggccattgctttactcttcttcttcatttgcgtctgtcggaataaccagaagtcatcgtcggcaccagtccagaggcaaccaaaacacgtcagaggtcaaaatgtggagatgtcaatgctgaatgcatataaacccaagagcaaggctaaagagctacctctttctgctgtacgctttatggaagaattgggtgagtgtgcctttggaaaaatctataaaggccatctctatctcccaggcatggaccatgctcagctggttgctatcaagaccttgaaagactataacaacccccagcaatggacggaatttcaacaagaagcctccctaatggcagaactgcaccaccccaatattgtctgccttctaggtgccgtcactcaggaacaacctgtgtgcatgctttttgagtatattaatcagggggatctccatgagttcctcatcatgagatccccacactctgatgttggctgcagcagtgatgaagatgggactgtgaaatccagcctggaccacggagattttctgcacattgcaattcagattgcagctggcatggaatacctgtctagtcacttctttgtccacaaggaccttgcagctcgcaatattttaatcggagagcaacttcatgtaaagatttcagacttggggctttccagagaaatttactccgctgattactacagggtccagagtaagtccttgctgcccattcgctggatgccccctgaagccatcatgtatggcaaattctcttctgattcagatatctggtcctttggggttgtcttgtgggagattttcagttttggactccagccatattatggattcagtaaccaggaagtgattgagatggtgagaaaacggcagctcttaccatgctctgaagactgcccacccagaatgtacagcctcatgacagagtgctggaatgagattccttctaggagaccaagatttaaagatattcacgtccggcttcggtcctgggagggactctcaagtcacacaagctctactactccttcagggggaaatgccaccacacagacaacctccctcagtgccagcccagtgagtaatctcagtaaccccagatatcctaattacatgttcccgagccagggtattacaccacagggccagattgctggtttcattggcccgccaatacctcagaaccagcgattcattcccatcaatggatacccaatacctcctggatatgcagcgtttccagctgcccactaccagccaacaggtcctcccagagtgattcagcactgcccacctcccaagagtcggtccccaagcagtgccagtgggtcgactagcactggccatgtgactagcttgccctcatcaggatccaatcaggaagcaaatattcctttactaccacacatgtcaattccaaatcatcctggtggaatgggtatcaccgtttttggcaacaaatctcaaaaaccctacagaattgactcaaagcaagcatctttactaggagacgccaatattcatggacacaccgaatctatgatttctgcagaactggactacaaggacgacgatgacaagtga

SEQ ID NO: 8 - protein, 945 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYRIDSKQASLLGDANIHGHTESMISAELDYKDDDDK

SEQ ID NO: 9 - DNA, 2856 bp, Homo sapiens
atggcccggggctcggcgctcccgcggcggccgctgctgtgcatcccggccgtctgggcggccgccgcgcttctgctctcagtgtcccggacttcaggtgaagtggaggttctggatccgaacgaccctttaggaccccttgatgggcaggacggcccgattccaactctgaaaggttactttctgaattttctggagccagtaaacaatatcaccattgtccaaggccagacggcaattctgcactgcaaggtggcaggaaacccaccccctaacgtgcggtggctaaagaatgatgccccggtggtgcaggagccgcggcggatcatcatccggaagacagaatatggttcacgactgcgaatccaggacctggacacgacagacactggctactaccagtgcgtggccaccaacgggatgaagaccattaccgccactggcgtcctgtttgtgcggctgggtccaacgcacagcccaaatcataactttcaggatgattaccacgaggatgggttctgccagccttaccggggaattgcctgtgcacgcttcattggcaaccggaccatttatgtggactcgcttcagatgcagggggagattgaaaaccgaatcacagcggccttcaccatgatcggcacgtctacgcacctgtcggaccagtgctcacagttcgccatcccatccttctgccacttcgtgtttcctctgtgcgacgcgcgctcccgggcacccaagccgcgtgagctgtgccgcgacgagtgcgaggtgctggagagcgacctgtgccgccaggagtacaccatcgcccgctccaacccgctcatcctcatgcggcttcagctgcccaagtgtgaggcgctgcccatgcctgagagccccgacgctgccaactgcatgcgcattggcatcccagccgagaggctgggccgctaccatcagtgctataacggctcaggcatggattacagaggaacggcaagcaccaccaagtcaggccaccagtgccagccgtgggccctgcagcacccccacagccaccacctgtccagcacagacttccctgagcttggaggggggcacgcctactgccggaaccccggaggccagatggagggcccctggtgctttacgcagaataaaaacgtacgcatggaactgtgtgacgtaccctcgtgtagtccccgagacagcagcaagatggggattctgtacatcttggtccccagcatcgcaattccactggtcatcgcttgccttttcttcttggtttgcatgtgccggaataagcagaaggcatctgcgtccacaccgcagcggcgacagctgatggcctcgcccagccaagacatggaaatgcccctcattaaccagcacaaacaggccaaactcaaagagatcagcctgtctgcggtgaggttcatggaggagctgggagaggaccggtttgggaaagtctacaaaggtcacctgttcggccctgccccgggggagcagacccaggctgtggccatcaaaacgctgaaggacaaagcggaggggcccctgcgggaggagttccggcatgaggctatgctgcgagcacggctgcaacaccccaacgtcgtctgcctgctgggcgtggtgaccaaggaccagcccctgagcatgatcttcagctactgttcgcacggcgacctccacgaattcctggtcatgcgctcgccgcactcggacgtgggcagcaccgatgatgaccgcacggtgaagtccgccctggagccccccgacttcgtgcaccttgtggcacagatcgcggcggggatggagtacctatccagccaccacgtggttcacaaggacctggccacccgcaatgtgctagtgtacgacaagctgaacgtgaagatctcagacttgggcctcttccgagaggtgtatgccgccgattactacaagctgctggggaactcgctgctgcctatccgctggatggccccagaggccatcatgtacggcaagttctccatcgactcagacatctggtcctacggtgtggtcctgtgggaggtcttcagctacggcctgcagccctactgcgggtattccaaccaggatgtggtggagatgatccggaaccggcaggtgctgccttgccccgatgactgtcccgcctgggtgtatgccctcatgatcgagtgctggaacgagttccccagccggcggccccgcttcaaggacatccacagccggctccgagcctggggcaacctttccaactacaacagctcggcgcagacctcgggggccagcaacaccacgcagaccagctccctgagcaccagcccagtgagcaatgtgagcaacgcccgctacgtggggcccaagcagaaggccccgcccttcccacagccccagttcatccccatgaagggccagatcagacccatggtgcccccgccgcagctctacatccccgtcaacggctaccagccggtgccggcctatggggcctacctgcccaacttctacccggtgcagatcccaatgcagatggccccgcagcaggtgcctcctcagatggtccccaagcccagctcacaccacagtggcagtggctccaccagcacaggctacgtcaccacggccccctccaacacatccatggcagacagggcagccctgctctcagagggcgctgatgacacacagaacgccccagaagatggggcccagagcaccgtgcaggaagcagaggaggaggaggaaggctctgtcccagagactgagctgctgggggactgtgacactctgcaggtggacgaggcccaagtccagctggaagctgactacaaggacgacgatgacaagtga

SEQ ID NO: 10 - protein, 951 aa, Homo sapiens
MARGSALPRRPLLCIPAVWAAAALLLSVSRTSGEVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRAPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMGILYILVPSIAIPLVIACLFFLVCMCRNKQKASASTPQRRQLMASPSQDMEMPLINQHKQAKLKEISLSAVRFMEELGEDRFGKVYKGHLFGPAPGEQTQAVAIKTLKDKAEGPLREEFRHEAMLRARLQHPNVVCLLGVVTKDQPLSMIFSYCSHGDLHEFLVMRSPHSDVGSTDDDRTVKSALEPPDFVHLVAQIAAGMEYLSSHHVVHKDLATRNVLVYDKLNVKISDLGLFREVYAADYYKLLGNSLLPIRWMAPEAIMYGKFSIDSDIWSYGVVLWEVFSYGLQPYCGYSNQDVVEMIRNRQVLPCPDDCPAWVYALMIECWNEFPSRRPRFKDIHSRLRAWGNLSNYNSSAQTSGASNTTQTSSLSTSPVSNVSNARYVGPKQKAPPFPQPQFIPMKGQIRPMVPPPQLYVPVNGYQPVPAYGAYLPNFYPVQIPMQMAPQQVPPQMVPKPSSHHSGSGSTSTGYVTTAPSNTSMADRAALLSEGADDTQNAPEDGAQSTVQEAEEEEEGSVPETELLGDCDTLQVDEAQVQLEADYKDDDDK

SEQ ID NO: 11 - DNA, 1311 bp, Homo sapiens
atggcccggggctcggcgctcccgcggcggccgctgctgtgcatcccggccgtctgggcggccgccgcgcttctgctctcagtgtcccggacttcaggtgaagtggaggttctggatccgaacgaccctttaggaccccttgatgggcaggacggcccgattccaactctgaaaggttactttctgaattttctggagccagtaaacaatatcaccattgtccaaggccagacggcaattctgcactgcaaggtggcaggaaacccaccccctaacgtgcggtggctaaagaatgatgccccggtggtgcaggagccgcggcggatcatcatccggaagacagaatatggttcacgactgcgaatccaggacctggacacgacagacactggctactaccagtgcgtggccaccaacgggatgaagaccattaccgccactggcgtcctgtttgtgcggctgggtccaacgcacagcccaaatcataactttcaggatgattaccacgaggatgggttctgccagccttaccggggaattgcctgtgcacgcttcattggcaaccggaccatttatgtggactcgcttcagatgcagggggagattgaaaaccgaatcacagcggccttcaccatgatcggcacgtctacgcacctgtcggaccagtgctcacagttcgccatcccatccttctgccacttcgtgtttcctctgtgcgacgcgcgctcccgggcacccaagccgcgtgagctgtgccgcgacgagtgcgaggtgctggagagcgacctgtgccgccaggagtacaccatcgcccgctccaacccgctcatcctcatgcggcttcagctgcccaagtgtgaggcgctgcccatgcctgagagccccgacgctgccaactgcatgcgcattggcatcccagccgagaggctgggccgctaccatcagtgctataacggctcaggcatggattacagaggaacggcaagcaccaccaagtcaggccaccagtgccagccgtgggccctgcagcacccccacagccaccacctgtccagcacagacttccctgagcttggaggggggcacgcctactgccggaaccccggaggccagatggagggcccctggtgctttacgcagaataaaaacgtacgcatggaactgtgtgacgtaccctcgtgtagtccccgagacagcagcaagatggggattctgtacatcttggtccccagcatcgcaattccactggtcatcgcttgccttttcttcttggtttgcatgtgcgactacaaggacgacgatgacaagtaa

SEQ ID NO: 12 - protein, 436 aa, Homo sapiens
MARGSALPRRPLLCIPAVWAAAALLLSVSRTSGEVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRAPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMGILYILVPSIAIPLVIACLFFLVCMCDYKDDDDK

SEQ ID NO: 13 - DNA, 20 bp, Artificial sequence
  top strand primer used to construct Ror1-flag
ctcatcaggatccaatcagg

SEQ ID NO: 14 - DNA, 65 bp, Artificial sequence
  bottom strand primer used to construct Ror1-flag
tgagcgcggccgctgctcacttgtcatcgtcgtccttgtagtccagttctgcagaaatcatagat

SEQ ID NO: 15 - DNA, 25 bp, Artificial sequence
  top strand primer used to construct Ror2-flag
agttccccagccggcggccccgctt

SEQ ID NO: 16 - DNA, 37 bp, Artificial sequence
  bottom strand primer used to construct Ror2-flag
tacgattctagatgtcaagcttccagctggacttggg

SEQ ID NO: 17 - DNA, 32 bp, Artificial sequence
  top strand primer used to construct Ror2-flag
gaccttggtaccatggcccggggctcggcgct

SEQ ID NO: 18 - DNA, 23 bp, Artificial sequence
  bottom strand primer used to construct Ror2-flag
tcgttcggatccagaacctccac

SEQ ID NO: 19 - DNA, 22 bp, Artificial sequence
  top strand primer used to construct Ror2-flag
gctcacaccacagtggcagtgg

SEQ ID NO: 20 - DNA, 61 bp, Artificial sequence
  bottom strand primer used to construct Ror2-flag
ctggaatctagatcacttgtcatcgtcgtccttgtagtcagcttccagctggacttgggcc

SEQ ID NO: 21 - DNA, 41 bp, Artificial sequence
  top strand primer used to construct Ror2KD-flag
ggctgtggccatcataacgctgatagacatagcggaggggc

SEQ ID NO: 22 - DNA, 41 bp, Artificial sequence
  bottom strand primer used to construct Ror2KD-flag
gcccctccgctatgtctatcagcgttatgatggccacagcc

SEQ ID NO: 23 - DNA, 25 bp, Artificial sequence
  top strand primer used to construct Ror2deltaC-flag
ccttctgccacttcgtgtttcctct

SEQ ID NO: 24 - DNA, 65 bp, Artificial sequence
  bottom strand primer used to construct Ror2deltaC-flag
gacctttctagattacttgtcatcgtcgtccttgtagtcgcacatgcaaaccaagaagaaaaggc

SEQ ID NO: 25 - DNA, 21 bp, Artificial sequence
  forward primer to identify human Ror1
gtcgactagcactggccatgt

SEQ ID NO: 26 - DNA, 27 bp, Artificial sequence
  reverse primer to identify human Ror1
catgtgtggtagtaaaggaatatttgc

SEQ ID NO: 27 - DNA, 27 bp, Artificial sequence
  probe to identify human Ror1
agcttgccctcatcaggatccaatcag

SEQ ID NO: 28 - DNA, 21 bp, Artificial sequence
  forward primer to identify human Ror2
cgtacgcatggaactgtgtga

SEQ ID NO: 29 - DNA, 21 bp, Artificial sequence
  reverse primer to identify human Ror2
caagcgatgaccagtggaatt

SEQ ID NO: 30 - DNA, 25 bp, Artificial sequence
  probe to identify human Ror2
ccctcgtgtagtccccgagacagca

SEQ ID NO: 31 - DNA, 21 bp, Artificial sequence
  forward primer to identify mouse Ror1
ccccgatttcccaattacatg

SEQ ID NO: 32 - DNA, 20 bp, Artificial sequence
  reverse primer to identify mouse Ror1
gccaatgaaaccacggatct

SEQ ID NO: 33 - DNA, 23 bp, Artificial sequence
  probe to identify mouse Ror1
cccgagccaagggattacacccc

SEQ ID NO: 34 - DNA, 23 bp, Artificial sequence
  forward primer to identify mouse Ror2
atccaagacctggacacaacaga

SEQ ID NO: 35 - DNA, 20 bp, reverse primer to identify mouse Ror2
gaaccccagtggcagtgatg

SEQ ID NO: 36 - DNA, 25 bp, Artificial sequence
  probe to identify mouse Ror2
tcagcccgttggtagccacacactg

SEQ ID NO: 37 - DNA, 20 bp, forward primer to identify mouse alkaline phosphatase
gagacccacggtggagaaga

SEQ ID NO: 38 - DNA, 20 bp, Artificial sequence
  reverse primer to identify mouse alkaline phosphatase
ggaggcatacgccatcacat

SEQ ID NO: 39 - DNA, 27 bp, Artificial sequence
  probe to identify mouse alkaline phosphatase
cggcgtccatgagcagaactacattcc

SEQ ID NO: 40 - DNA, 19 bp, Artificial sequence
  forward primer to identify mouse osteocalcin
cggccctgagtctgacaaa

SEQ ID NO: 41 - DNA, 22 bp, Artificial sequence
  reverse prime to identify mouse osteocalcin
gccggagtctgttcactacctt

SEQ ID NO: 42 - DNA, 24 bp, Artificial sequence
  probe to identify mouse osteocalcin
ccttcatgtccaagcaggagggca

SEQ ID NO: 43 - DNA, 2000 bp, Homo sapiens
gggctgggagtctggagtccatgacctgctgggacatactttgggctgcattttttcccccttctaatgtctcagctctcagggatctgcttttcctggtgccaaggagtatggatgaaattttgtccctcctccatgcacccctataaaacaaatatgatgagaaattaatattttccaaatggtgacctaacctttgttcattaattgagctcctattgcattccaggtactgttagaggccagcaagatacctagatgattgagacagagtcctcgccctccatgaagtgactgttcattttaaaaccctaaagctgggttcttagaggctactgcaacagcaacaactcccgagtgagtgcttatcatgtgccaggctctggttacaggctttaactctctcagtggaaacagcctgagtgctggcttggctaccccatgtctgtgagactgtgggcaagtcatttactttccctttgagtttcaatttctaaaactgtaatatggggataatatatgtaaaacagctaagaagtatatacttttaatgactctttttagtgtcaatggaggatttgtgagcaaatgcatataaaacacaaggcaaacaggaggtatttagtaagtggccaagtgattattgagagactatgggcttcttcctgttcctccagagcaagactgacaggtgtcctattacccttgcatcttcagagcctagagcacagccctgaaaacagcaggcgctcaccactttttatgatcaaaggcagtctctacagggaaagtcaaatcattatgggtatttatgaattacaaaatccataaagttaaaccaggagaaatggacaaaaacacactcattgtagaggtctttaatgatcccttggctcatggcagagcagctagacaaaaaacaaacaaacaaacaaacatgcaaagattcagaaggcctgaaaaaataatgaatacaaagaagataaatcgatacagtattctatcttcacattcggaagacaaaaacacctttcaaaaaaccatgggacattggcaaaaataaccacagataatcctcaataaatcttgccaggaggcagtacagatgacattgaattcagtgagactacaaattaaagacaaaaaggcgaagtataaattacttgtgtaagaactctctcaactcttgggtccaagaagaaagcaaaagggaaattgtatgatagcaataaatgtgccattacccagagtcacacagctaacagtgacacagctgaggctgctagaacctgggtctactgtcgtccagccacgaactggcttgagatcccggataagtctctctgagcctcggtttccccttctgtcaagtagcctcgaaggtccccggcgctcggttcggtggcgagtttgaggagtgtgggggaggggaggggagggagcgtgcccgagacgcgggagccgcgaccgctttctgcgaagtcggggagggtgcgggcgttgagaggctgcagcagagggcgctgggtcgccagcctgggccgcgtctcccattggtcggggctggggcgctgggctggagagttggtggaaagtgacaagttgagcgagagagggagcgtggagagctggagcagccgccaccgccgccgccgagggagccccgggacggcagcccctgggcgcagggtgcgctgttctcggagtccgacccagggcgactcacgcccactggtgcgacccggacagcctgggactgacccgccggcccaggcgaggctgcagccagagggctgggaagggatcgcgctcgcggcatccagaggcggccaggcggaggcgagggagcaggttagagggacaaagagctttgcagacgtccccggcgtcctgcgagcgccagcggccgggacgaggcggccgggagcccgggaagagcccgtggatgttctgcgcgcggcctgggagccgccgccgccgccgcctcagcgagaggagga

SEQ ID NO: 44 - DNA, 2000 bp, mouse
  misc_feature (1848)..(1867): n is A, C, T or G
ctttataaaagttgccctggtcttggtttcatggcaatgaaaccctcaataagtggcttgggtggaattatttgtgaatttggtctttggcagtaacctgtctgggcttcatatgggtgcactcctgcagcacaggggaaggtttccaaactatagcaacagataacatgactactggggcatgtgtctgtggtgggagtcctgctactctacctgaagccctgtggggttgtggattgacaatgacaaaaacagtgccatctttacctggtattaaaaagttactaaattctttttttctctctttttttctcccctcccccactttttaaatttttgtctttttttttttggagtctgtctcattatgtaggtctggatatcctggagctgattctgcagcccaggctggtctcagactcacagagctctgcctgcctctgcctcccaagttcatcaaagacacatgccaccgtgactagctacaaataactcttaaagtcataaaaaaaaccaagactacaggcaacccaattaaaaccaaatgtggggctggagctatgccttcgtgggtgagtgcttgcctaacattcgagaggttctgggctcagtctccagcaccatctgcacatgcctgcaatccaagcgctcaggaggcaaaggcagggtacactcgaggttatctttggctacataacaactttgaagttagtctaggattcatgtgcctctgtttaaaaacaacatcaacaacaatgaaaacaactgaaccaaataagaccaaccttggtccttggcaacaccctgtcaaagaggagtgatgctgccggggctggagagacagctggctcagcagttaagagcaccgactgctcttgcagaggaccctggttcagtccccagcactcccaacgaagctcacaatcatctgcaaccttagcttaaggggatgtaaagccctcttctgacctcctctgtcactgcacacatctaggtcacagccatgcacgcaggtactcacacatgcatgtaaaataaaaatgagcacatctcaaagcagcaacaacaaaggctggggatgtagatcaaactgtttgcctaatatgtagaagaccttgaattaaactacccagcctcctgcctttcaaatatttgaagtgatatttcccagaagaagtttaggctgttggtgaacaaaggaaagatactaccgtgcaaaggcacataacattcattaggaaaatacacgttaacacatacattgtcgacgccctggtctccagtgtgttcttgggatactccaggacccctcgctcaagctctgccccaggacccttcacttccctgatgtgtgctggctgttgtcatagccttggttggctcccatcctactaccctcattaatggggcgtgtgctgactttttgctagattcccggagggtcctgatcctcccaaggggcttggcaggacgcccatggggacgcagccggctatattctggaaaatctgtgttcgaagaccatacctcatctctcactttgggcccaggatttcgaagaactgacaactgaattcccctcgtggggcttcatcaagggacgcctagttaatgatttgtgtggtccaggtccccaagtttccaggcttctgatggtgggtggcaacactgctgcaagagcagatgaaccggaggctccacctttgtgcctagaagtcccggaggcgctggcgggagggcgggtgctgcaggcggctccctggacgcaggggcggacccggctccttcccgctcactcgggtcgctgcccttgagtcactccggcagagggcgcccggcgccggcctnnnnnnnnnnnnnnnnnnnngccctcgaagggaccagcctgcgaagcgcgaggaaggaagaagcggacgcgtggcggggaggggttgaggcgccccggcccgaagcccaaagccgagcgcgcggtaggcgaccgggcacccacggcccgcagc

SEQ ID NO: 45 - DNA, 41 bp, Artificial sequence
  top strand primer used to construct the 5′ portion of Notch2IC (1-782)
catatgaattcatgaccaagcatggctctctctggctgcct

SEQ ID NO: 46 - DNA, 23 bp, Artificial sequence
  bottom strand primer used to construct the 5′ portion of Notch2IC (1-782)
cgcttggcagttgatcagttctg

SEQ ID NO: 47 - DNA, 25 bp, Artificial sequence
  top strand primer used to construct the 3′ portion of Notch2IC (783-2307)
gaatggtggcagaactgatcaactg

SEQ ID NO: 48 - DNA, 39 bp, Artificial sequence
  bottom strand primer used to construct the 3′ portion or Notch2IC (783-2307)
gatatgcggccgccgcataaacctgcatgttgttgtgtg